(19) United States
(12) Patent Application Publication
(10) Pub. No.: US 2025/0066738 A1
(43) Pub. Date: Feb. 27, 2025

(54) INDUCED PLURIPOTENT STEM CELLS (IPSC), T-CELL COMPOSITIONS AND METHODS OF USE

(71) Applicant: Dana-Farber Cancer Institute, Inc., Boston, MA (US)

(72) Inventors: Jooeun Bae, West Roxbury, MA (US); Nikhil Munshi, Needham, MA (US); Kenneth Anderson, Wellesley, MA (US)

(21) Appl. No.: 18/689,977

(22) PCT Filed: Sep. 6, 2022
(86) PCT No.: PCT/US2022/075997

§ 371 Date: Mar. 7, 2024

Related U.S. Application Data
(60) Provisional application No. 63/241,428, filed on Sep. 7, 2021.
(60) Provisional application No. 63/348,123, filed on Jun. 2, 2022.

(51) Int. Cl. C12N 5/074; A61K 35/12; A61P 35/00

(52) U.S. Cl. CPC C12N 5/0696; A61K 35/12; A61P 35/00; C12N 2506/115; C12N 2510/00

(57) ABSTRACT

Aspects of the invention are drawn to induced pluripotent stem cells (iPSC) and re-differentiated T cells from iPSC, related compositions, and methods of using the same.

All patents, patent applications and publications cited herein are hereby incorporated by reference in their entirety. The disclosures of these publications in their entireties are hereby incorporated by reference into this application to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### CROSS-REFERENCE TO RELATED APPLICATIONS

The present application is the § 371 U.S. National Stage of International Application No. PCT/US2022/075997, filed Sep. 6, 2022, which claims the benefit of priority to United States Provisional Application Nos. 63/241,428, filed Sep. 7, 2021, and 63/348,123, filed Jun. 2, 2022, the contents of which are incorporated herein by reference.

### SEQUENCE LISTING

This application contains a Sequence Listing electronically submitted to the United States Patent and Trademark Office via Patent Center as an XML file entitled “5031461-000128-WO1_SL” having a size of 9.55 kilobytes and created on Nov. 3, 2022. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### FIELD OF THE INVENTION

Aspects of the invention are drawn to induced pluripotent stem cells (iPSC) and re-differentiated T cells from iPSC, related compositions, and methods of use thereof.

### BACKGROUND

Induced pluripotent stem cells (iPSCs) are stem cells produced from somatic cells. In some examples, introduction and expression of four genes (e.g., c-MYC, OCT3/4, SOX2 and KLF4) can reprogram somatic cells into iPSCs. Multiple types of somatic cells have reprogrammed into iPSCs. Thus, iPSCs have a variety of medical uses.

### SUMMARY

Disclosed are induced pluripotent stem cells (iPSCs) that re-differentiate to at least one of a CD8⁺ cytotoxic T lymphocyte (CTL) (iPSC [CD8⁺ T cell]), a lymphocyte that does not express CD3 (iPSC [CD3⁻ lymphocyte]) or a non-lymphocyte (iPSC [non-lymphocyte]). The iPSCs can re-differentiate to one of a CD8⁺ cytotoxic T lymphocyte (CTL) (iPSC [CD8⁺ T cell]), a lymphocyte that does not express CD3 (iPSC [CD3⁻ lymphocyte]) or a non-lymphocyte (iPSC [non-lymphocyte]).

The iPSCs can re-differentiate to a CD8⁺CTL (iPSC [CD8⁺ T cell]) and not to a lymphocyte that does not express CD3 (iPSC [CD3⁻ lymphocyte]) or a non-lymphocyte (iPSC [non-lymphocyte]). The iPSCs can be specific for an antigen. The antigen can be a tumor antigen, including a tumor-associated antigen (TAA) or a tumor-specific antigen (TSA). The antigen can be B-cell maturation anigen (BCMA). The antigen can be BCMA₇₂₋₈₀ (YLMFLLRKI (SEQ ID NO: 1)). The iPSCs can be re-programmed from a CD8⁺CTL, which can be specific for an antigen. The iPSCs can have a normal karyotype, express SSEA-4 and TRA-1-60, differentiate into ectoderm, mesoderm and endoderm, retain alkaline phosphate during colony formation, or a combination thereof.

The iPSCs that re-differentiate to a CD8⁺CTL (iPSC [CD8⁺ T cell]) can have increased expression of the genes FOXF1, GZMB, ITGA1, TBX3, MX1, TNFRSF9, CD1A, LCK, LTB, IFIT3, TNFSF10 and/or A2M as compared to iPSC that re-differentiate to the lymphocyte that does not express CD3 (iPSC [CD3⁻ lymphocyte]).

The iPSCs that re-differentiate to a CD8⁺CTL (iPSC [CD8⁺ T cell]) can have decreased expression of the genes TGFBR3, CD37 and/or SlPRl as compared to iPSC that re-differentiate to the lymphocyte that does not express CD3 (iPSC [CD3⁻ lymphocyte]).

The iPSCs that re-differentiate to a CD8⁺CTL (iPSC [CD8⁺ T cell]) can have increased expression of the genes TBX3, ZNF683, FOXF1, GZMB, IL7R, A2M and/or SORL1 as compared to iPSC that re-differentiate to the non-lymphocyte (iPSC [non-lymphocyte]).

The iPSCs that re-differentiate to a CD8⁺CTL (iPSC [CD8⁺ T cell]) can have decreased expression of the genes TGFBR3, GDF3, BLNK, FRRS1, KLF2, NCF2 and/or KDR as compared to iPSC that re-differentiate to the non-lymphocyte (iPSC [non-lymphocyte]).

The iPSCs that re-differentiate to a CD8⁺CTL (iPSC [CD8⁺ T cell]) can have increased expression of the genes CX3CR1, CD3D, CD1A, CDH5, ILR7, PLVAP, LEF1, A2M, NCR2, CCNB2, ORC6 and/or NUSAP1 as compared to hematopoietic progenitor cells (HPC), which are CD34⁺CD43⁺/CD14⁻ CD235a⁻, from the iPSC.

The iPSCs that re-differentiate to a CD8⁺CTL (iPSC [CD8⁺ T cell]) can have decreased expression of the genes DNTT, LAG3, KLF2, CD37, SELL and SORL1 as compared to hematopoietic progenitor cells (HPC), which are CD34⁺CD43⁺/CD14⁻ CD235a⁻, from the iPSC.

The iPSCs that re-differentiate to a CD8⁺CTL (iPSC [CD8⁺ T cell]) can have increased expression of the genes TBX3, HOXA11, IRF4, PIK3C2B, KLF15, IL-12B, MAPK4, ITLN 1/2, TRIM6 and/or EDA2R as compared to iPSC that re-differentiate to a lymphocyte that does not express CD3 (iPSC [CD3⁻ lymphocyte]) and iPSC that re-differentiate to a non-lymphocyte (iPSC [non-lymphocyte]).

The iPSCs that re-differentiate to a CD8⁺CTL (iPSC [CD8⁺ T cell]) can have decreased expression of the genes RPS6KA2, CDK3, YEPL4, BATF2, BTN3A1, BTN3A1, USP44, CD70, ZXDA, FGFR1, NPM2, GGN, SPAG1, CATSPER2, N4BP3, P2RY14, NLGN2, SHC2, GRASP, AMIGO2, TBC1D32, CACNA1A, SLC6A9, HEYL, NEURL, RAB39B, ANK1, PSD, LRRK1, RUNX2, CXCL5, SEMA7A, JDP2, PLA2G6, MAP3K9, PIPOX and/or TNFRSF6B as compared to iPSC that re-differentiate to a lymphocyte that does not express CD3 (iPSC [CD3⁻ lymphocyte]) and iPSC that re-differentiate to a non-lymphocyte (iPSC [non-lymphocyte]).

Disclosed are cytotoxic T cells (CTL) re-differentiated from the iPSC as above. The CTL can have an antigen-specific (e.g., BCMA), MHC-restricted proliferation response and/or an antigen-specific MHC-restricted cytotoxic response. The CTL can be a memory CD8⁺CTL (CD45RO⁺). The memory CD8⁺ CTL can be a central memory CTL (CCR7⁺ CD45RO⁺). The memory CD8⁺CTL can be an effector memory CTL (CCR7⁻ CD45RO⁺).

Disclosed are compositions of the iPSC as above, and/or CTL re-differentiated from the iPSC as above.

Disclosed are methods for treating a cancerous or precancerous condition in a subject by administering iPSCs as above, CTL re-differentiated from the iPSC as above, and/or compositions of the iPSCs and/or CTL re-differentiated from the iPSC. The cancerous condition can be a blood borne cancer, like myeloma. The precancerous condition can be smildering myeloma or monoclonal gammopathy of underdetermined significance.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1A shows a schematic of an exemplary protocol used in one embodiment of the present invention.

FIG. 1B shows example photomicrographs (100×) of BMA-specific iPSC clones taken with an inverted microscope at days 8, 12, and 16 following transduction.

FIG. 1C shows example self-renewal capacity and the level of cell proliferation for BCMA-specific iPSC clones 1, 2, and 3 (lines A, C, & D, respectively) vs. control EBV-specific iPSCs (line B) on weeks 0, 1, 2, 3, 4 and 5 following transduction.

FIG. 1D shows example pluripotency potential of BCMA-specific iPSC clones (#1-4) measured by expression of stem cell markers, SSEA-4 and TRA-1-60 in BCMA-specific iPSC clones and control EBV-specific iPSC clone, by flow cytometry.

FIG. 1E shows example pluripotency potential of BCMA-specific iPSC clones (#1-3) measured by expression of representative germ layer markers, such as SOX-17 on endoderm, brachyury on mesoderm, and Pax-6 on ectoderm in the BCMA-iPSC clones and control EBV-specific iPSC clone, by flow cytometry.

FIG. 1F shows example clonogenic and self-renewal potential of BCMA-specific iPSC measured by upregulation of alkaline phosphatase activity in BCMA-specific iPSC, EBV-specific iPSC, and control T lymphocytes using immunohistochemistry.

FIG. 1G shows example genomic stability and normal karyotype of BCMA-specific iPSC clones (#1-3) measured by cytogenetic analyses of chromosomes based on Giemsa banding (G-banding) patterns.

FIG. 1H shows example expression of Sendai virus residue following the reprograming process in BCMA-iPSC, EBV-iPSC, GPC3 16-1-iPSC. Sendai virus CytoTune2.0 supernatant was used as a positive control.

FIG. 2A-E shows example polarization into mesoderm germ layer during BCMA-specific embryoid body formation from BCMA-specific iPSC.

FIG. 2A provides example photomicrograph evaluation of BCMA-specific iPSCs and -embryoid bodies (EBs) (day 11) and positive control EBV-specific iPSCs and -EBs (day 11) (100×) taken by inverted microscope.

FIG. 2B shows examples of gradual upregulation in genes associated with mesoderm development of BCMA-specific Clone #1 iPSC vs. EB during embryoid body formation on days 2, 4, and 7 using ScoreCard analysis, which determined the fold change in gene expression relative to an undifferentiated reference set.

FIG. 2C shows examples of gradual upregulation in genes associated with mesoderm development of BCMA-specific Clone #2 iPSC vs. EB during embryoid body formation on days 2, 4, and 7 using ScoreCard analysis, which determined the fold change in gene expression relative to an undifferentiated reference set.

FIG. 2D shows examples of no specific regulation in genes associated with germ layer development of CD8⁺ T cells (N=2; Donor 1, Donor 2) representing somatic cell type using ScoreCard analysis relative to an undifferentiated reference set.

FIG. 2E provides an example gene expression profile summary associated with primary germ layers development in BCMA-specific iPSC, embryoid body formation (measured on day 2, 4 and 7) and CD8⁺ T lymphocytes, to that of the undifferentiated reference set.

FIGS. 3A-K show example differentiation of BCMA-specific embroyoid body-derived HPC into rejuvenated antigen-specific CD8+CTL with mature T cell phenotype.

FIG. 3A shows example sorting of reprogramed hematopoietic progenitor cells (HPC; CD34⁺CD43⁺/CD14⁻ CD235a⁻) in BCMA-specific embryoid body (48-59%) formed from BCMA-specific iPSC clones (#1-3) and EBV-specific embryoid body (82%) using a FACS Aria flow cytometer.

FIG. 3B shows example changes in cell numbers over a three-week period in BCMA-specific clones (e.g., cell expansion during differentiation of HPC) and an EBV-specific clone.

FIG. 3C shows example gradual phenotypic differentiation of HPC into CD3⁺ CD8⁺ T cells over a three-week period in the presence of retronectin/Fc-DLL4 signal and re-differentiation media. No CD3⁺ T cell differentiation was observed when the progenitor cells were not exposed to the retronectin/Fc-DLL4 signaling but cultured in re-differentiation media alone.

FIG. 3D shows example phenotypic characterizations of T cells differentiated from BCMA-specific iPSC at day 21 by flow cytometric analysis.

FIG. 3E shows an example uniform pattern of phenotype of the differentiated BCMA-specific iPSC-T cells (clones #1-3) with (1) high frequency (˜90%) of T cells and CTL markers (CD3, CD45, CD8α, CD8β, CD7) and T cell receptor (TCRαβ), which are constitutively expressed on normal T cells, (2) lower frequency (˜40%) of CD5⁺ cells, and (3) minimum level (<5%) of T helper cells (CD4⁺), NK cells (CD16⁺, CD56⁺) and TCRγδ T cells. In addition, HLA-A2 molecule expression was maintained highly upon re-differentiation of iPSC to T cells. Data are shown as averages±standard deviations. Data were obtained using flow cytometry.

FIG. 3F provides an example histological image (100×) showing morphological characteristics of BCMA-specific iPSC-T cells compared to normal T lymphocytes.

FIG. 3G shows example expression of activation and co-stimulatory markers as well as immune checkpoints or induction of regulatory T cells in BCMA-specific iPSC-T cells (day 21 differentiation). Data obtained by flow cytometry.

FIG. 3H shows example graphical representations of the FIG. 3G data.

FIG. 3I shows results from evaluation of immune suppressor cells during the process of T cell differentiation in BCMA-specific iPSC-T cells from iPSC clone # and iPSC clone #2. No induction of CD3⁺Treg and CD4⁺Treg (CD25⁺FOXP3⁺) in BCMA-specific iPSC-T cells differentiated from iPSC clone #1 or iPSC clone #2, measured by flow cytometric analyses, is shown.

FIG. 3J shows results from further investigation of T cell differentiation potential upon multiple subcloning of BCMA-specific iPSC-T cells (subclones A, B, C). Maintenance of T cell differentiation capacity by subclone (A, B, C) of BCMA-specific iPSC is shown.

FIG. 3K shows flow cytometric evaluation by the level of T cell differentiation from the parent BCMA-iPSC as fresh cells, BCMA-iPSC upon cryopreservation for 8 months and BCMA-iPSC upon cryopreservation for 16 months. Maintenance of T cell differentiation capacity of BCMA-specific iPSC after long-term cryopreservation (8 months, 16 months) is shown.

FIGS. 4A-F shown example specific transcriptional regulation pathway of reprogrammed BCMA-specific HPC in their CD8⁺ CTL commitment.

FIG. 4A shows example principle component analysis determining the transcriptional variance within or across the HPC of BCMA-specific iPSC groups with normalized gene expression values in one embodiment. The data show different commitment pathways in comparison with HPC of PBMC.

FIG. 4B provides an example hierarchical cluster analyses using the top 1,000 variably expressed genes across a dataset in one embodiment. Clusters 1 and 4—upregulated (Clusters 1) or downregulated (Cluster 4) in HPC of all groups of iPSC compared to HPC of PBMC, Clusters 2 and 3—downregulated (Cluster 2) or upregulated (Cluster 3) in HPC of iPSC [CD8⁺ T cells] compared to HPC of iPSC [CD3⁻ lymphocytes] and HPC of iPSC [non-lymphocytes].

FIG. 4C provides an example graphical representation comparing the number of differentially expressed genes between iPSC [CD8⁺ T cells] and iPSC [CD3⁻ lymphocytes], between iPSC [CD8⁺ T cells] and iPSC [non-lymphocytes], and between iPSC [CD8⁺ T cells] and CD34⁺HSC in one embodiment. The data show upregulated (log fold change >2) or downregulated (log fold change <−2) genes, in HPC of iPSC [CD8⁺ T cells] compared to HPC of iPSC [CD3⁻ lymphocytes], HPC of iPSC [non-lymphocytes] or HPC of PBMC

FIG. 4D shows upregulated (top left) and down-regulated (bottom left) genes in iPSC [CD8⁺ T cells] compared to iPSC [CD3⁻ Lymphocytes] in an embodiment.

FIG. 4E shows upregulated (top left) and down-regulated (bottom left) genes in iPSC [CD8⁺ T cells] compared to iPSC [non-lymphocytes] in one embodiment.

FIG. 4F shows upregulated (top left) and down-regulated (top right) genes in iPSC [CD8⁺ T cells] compared to CD34⁺HPC in an embodiment.

FIGS. 5A-B show example transcriptional profiles of HPC in BCMA-specific iPSC with a distinct commitment pathway.

FIGS. 5A and 5B show transcriptional profiles of hematopoietic progenitor cells (HPC) from BCMA-specific iPSC in one embodiment.

FIG. 5A, left panel, shows a Venn diagram of commonly expressed or uniquely expressed genes that are upregulated in (1) iPSC [CD8⁺ T cells] vs. iPSC [CD3⁻ lymphocytes], (2) iPSC [CD8⁺ T cells] vs. iPSC [non-lymphocytes], and (3) iPSC [CD8⁺ T cells] vs. CD34⁺HSC (PBMCs).

FIG. 5A, right panel, shows a Venn diagram of commonly expressed or uniquely expressed genes that are downregulated in (1) iPSC [CD8⁺ T cells] vs. iPSC [CD3⁻ lymphocytes], (2) iPSC [CD8⁺ T cells] vs. iPSC [non-lymphocytes], and (3) iPSC [CD8⁺ T cells] vs. CD34+HSC (PBMCs).

FIG. 5B shows example data from evaluation of genes for their specific enrichment functional terms via GO annotation analysis. The data show functionalterms of “commonly” expressed genes in three separate cohort analyses for HPC in (1) iPSC [CD8⁺ T cells] vs. iPSC [CD3⁻ lymphocytes], (2) iPSC [CD8⁺ T cells] vs. iPSC [non-lymphocytes], and (3) iPSC [CD8⁺ T cells] vs. PBMC, evaluated by GO annotation.

FIGS. 6A-G show examples of high proliferation and anti-tumor activities of BCMA-specific iPSC-T cells against multiple myeloma cells in an antigen-specific and HLA-A2-restricted manner.

FIG. 6A provides example data from a CFSE-based proliferation assay from BCMA-specific iPSC T cells (CD3⁺, CD8⁺) in response to multiple myeloma stimulator cells in BCMA-specific and HLA-A2-restricted manner as measured by CFSE-based assay, under one embodiment.

FIG. 6B shows example data from experiments investigating the cytotoxic activities of BCMA specific iPSC Clone #1 T cells (CD8⁺) and Th1-type cytokine production in response to BCMA+/HLA-A2+U266 MM cells and RPMI (negative control) in an embodiment.

FIG. 6C shows data from experiments investigating the cytotoxic activities of BCMA specific iPSC Clone #2 T cells and Th1-type cytokine production in response to U266 MM cells and RPMI (negative control) in an embodiment.

FIG. 6D shows example CD107a upregulation (top left), IFN-γ production (top right), IL-2 production (bottom left), and TNF-α production in BCMA-specific iPSC T cells in response to U266 MM cells, RPMI MM cells, and MDA-MB231 breast cancer cells in one embodiment.

FIG. 6E shows example data from experiments investigating the functional activities of BCMA specific iPSC Clone #1 T cells against primary HLA-A2⁺CD138⁺ tumor cells (HLA-A2⁻ control) isolated from MM patients A, B, C and D in an embodiment.

FIG. 6F shows example data from experiments investigating the functional activities of BCMA specific iPSC Clone #2 T cells against primary HLA-A2⁺CD138⁺ tumor cells (HLA-A2⁻ control) isolated from MM patients A, B, C and D in an embodiment.

FIG. 6G shows example anti-tumor activities and immune responses to CD138⁺ tumor cells from HLA-A2⁺ MM and HLA-A2⁻ MM patients in BCMA-specific T cells differentiated from iPSC clone #1 (top) and iPSC clone #2 (bottom) in an embodiment.

FIGS. 7A-D shows examples of specific proliferation of BCMA-specific iPSC-T cells to cognate heteroclitic BCMA₇₂₋₈₀ (YLMFLLRKI (SEQ ID NO: 1)) peptide with a display of TCRαβ clonotype.

FIG. 7A shows example results from flow cytometric analysis of BCMA-specific iPSC-T cell proliferation in response to (1) iPSC-T cells alone, (2) iPSC-T cells stimulated with no peptide pulsed T2 or K562-A*0201 cells, (3) iPSC-T cells stimulated with HLA-A2-specific and relevant BCMA peptide (heteroclitic BCMA₇₂₋₈₀; YLMFLLRKI (SEQ ID NO: 1)) pulsed T2 or K562-A*0201 cells, and (4) iPSC-T cells stimulated with HLA-A2-specific but irrelevant HIV peptide (HIV-Gag₇₇₋₈₅; SLYNTVATL (SEQ ID NO: 2)) pulsed T2 or K562-A*0201 cells in one embodiment. Measurements obtained in CFSE-based assay.

FIG. 7B shows example results from flow cytometric analysis of BCMA-specific iPSC-T cell proliferation in response to T2 cells or T2 cells/BCMA Peptide stimulator (YLMFLLRKI (SEQ ID NO: 1)) at 5, 6, and 7 days in one embodiment.

FIG. 7C shows example results from flow cytometric analysis of BCMA-specific iPSC-T cell proliferation in response to U266 MM cells expressing HLA-A2, with or without additional pulse of the HLA-A2-specific heteroclitic BCMA₇₂₋₈₀ (YLMFLLRKI (SEQ ID NO: 1)) peptide at 4, 5, and 6 days in an embodiment.

FIG. 7D shows an example schematic of single cell-based TCR sequencing in complementarity-determining regions (CDR) important in diversity of antigen specificities by lymphocytes in a 96 well plate under one embodiment. A single cell from BCMA iPSC-T cells were sorted into a 96-well plate and processed using TCRseq procedure. Yellow indicates single cells that are part of a clone with a specific paired alpha/beta clonotype and gray indicates wells where the sequencing results did not pass quality control (QC). Unique clonotype TCRα and TCRβ sequences were identified based on single cell-based sequencing in CDR3 region of cognate heteroclitic BCMA₇₂₋₈₀ peptide-specific Tetramer⁺ CTL. Figure discloses SEQ ID NOS 9-10, respectively, in order of appearance.

FIG. 8A-E shows example CD45RO⁺ memory CD8+CTL as predominant subset demonstrating anti-myeloma activity by BCMS-specific iPSC-T cells.

FIG. 8A shows results from phenotypic characterization of BCMA-specific iPSC-T cells for certain cell subsets within the CD8⁺CTL from iPSC clone 1, clone 2 and clone 3 in one embodiment (top). Also shown is an example summary of expansion of memory CD8+CTL in BCMA-specific iPSC-T cells differentiated from the iPSC clones (N=3)(bottom).

FIG. 8B provides an example graphical representation of the percentage of Naïve, Central Memory, Effector Memory and Terminal Effector CD8+ T cell subsets in BCMA-specific iPSC−CD8+T from clone 1, 2, and 3 under an embodiment (N=3). The data show an expansion in central memory and effector memory CTL subsets.

FIG. 8C provides example results from investigation of functional anti-tumor activity and immune response of naïve, central memory, effector memory, and terminal effector cells in BCMA-specific iPSC-T cells to U266 MM cells under one embodiment. The data show high anti-myeloma activity by the central memory CTL subset, followed by the effector memory CTL subset.

FIG. 8D provides an example summary analysis of CD107a degranulation (top left), IFN-γ production (top right), IL-2 production (bottom left), and TNF-α production (bottom right) in response to U266 MM cells in BCMA-specific iPSC-CD8⁺ T cells (CD45RO⁺ memory CTL compared to CD45RO⁻ non-memory CTL) in an exemplary embodiment.

FIG. 8E provides an example summary analysis of CD107a degranulation (top left), IFN-γ production (top right), IL-2 production (bottom left), and TNF-α production (bottom right) in response to U266 MM cells in naïve, central memory (CM), effector memory (EM) and terminal effector (TE) subsets of BCMA-specific iPSC-CD8⁺ T cells in an exemplary embodiment. The data show high anti-myeloma activity by the central memory CTL subset, followed by the effector memory CTL subset.

FIGS. 9, 10, 11 and 12 show example results of validation of quality of RNA purified from each group of hematopoietic progenitor cells by viper output analyses.

FIG. 9 shows results from studies to validate and confirm the quality of RNA purified from each HPC by viper output analyses under one embodiment. The figure shows example data validation by Read alignment.

FIG. 10 shows results from studies to validate and confirm the quality of RNA purified from each HPC by viper output analyses under one embodiment. The figure shows example data validation by Gene body coverage.

FIG. 11 shows results from studies to validate and confirm the quality of RNA purified from each HPC by viper output analyses under one embodiment. The figure shows example data validation by Feature distribution.

FIG. 12 shows results from studies to validate and confirm the quality of RNA purified from each HPC by viper output analyses under one embodiment. The figure shows example data validation by number of Genes Detected.

FIG. 13 provides data showing key stem cell markers on BCMA-iPSC in one embodiment. High expression of stem cell markers [SSEA-4 and TRA-1-60; 99%] and alkaline phosphatase on BCMA-specific iPSC was detected, compared to BCNA-specific CD8⁺ CTL or CD3⁺ T lymphocytes.

FIG. 14 shows enrichment of BCMA-specific CD34+HPC under one embodiment. In this embodiment, reprogramed hematopoietic progenitor cells (HPC; CD34⁺CD43⁺/CD14⁻ CD235a⁻/Live cells gated) in EBV-specific embryoid body or BCMA-specific embryoid body were sorted for T cells differentiation.

FIG. 15 shows phenotypes of BCMA iPSC-T cells under one embodiment. Differentiation of iPSC-T cells from the progenitor cells shows high yields of TCRab⁺, CD45⁺, CD8ab⁺, HLA-A2⁺, CD7⁺ and T cells activation markers, without induction of immune checkpoint molecules.

FIG. 16 shows a-Tumor activity of BCMA iPSC-T cells under one embodiment. In this embodiment, BCMA-specific iPSC-T cells were rejuvenated memory CD8⁺ T cells with high level of anti-tumor activities to MM cell lines and MM patients' bone marrow cells in HLA-A2 restricted manner.

### DETAILED DESCRIPTION OF THE INVENTION

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Disclosed here are antigen-specific induced pluripotent stem cells (iPSC), and re-differentiated cells from the iPSC, which can be used for treatment of patients, in some examples, with cancer. In some examples, the antigen used to reprogram somatic cells to an iPSC includes a cellular antigen or a tumor antigen. In some examples, the antigen includes tumor-associated antigen (TAA) or tumor-specific antigen (TSA). In some examples, the antigen includes B-cell maturation antigen (BCMA).

Disclosed are heteroclitic immunogenic BCMA₇₂₋₈₀ peptide (YLMFLLRKI (SEQ ID NO: 1))-specific induced pluripotent stem cells (iPSC) and re-differentiated T cells from the iPSC. In some examples, the iPSC are established from BCMA-specific cytotoxic T lymphocytes (CTL) that are reprogrammed to obtain the iPSC. The iPSCs generally have a normal karyotype, express stem cell markers including SSEA-4 and TRA-1-60, differentiate into ectoderm, mesoderm and endoderm, and/or retain alkaline phosphatase during colony formation.

The iPSC may re-differentiate into CD8+ T cells (iPSC [CD8⁺ T cells]), CD3-lymphocytes (iPSC [CD3⁻ lymphocytes]) (including B cells, NK or NKT cells), and/or non-lymphocytes (iPSC [non-lymphocytes]) (including monocytes and granulocytes). In some examples, iPSC clones have been identified that predominately differentiate into CD8+ T cells, CD3⁻ lymphocytes or non-lymphocytes. In some examples, individual iPSC clones formed hematopoietic progenitor cells (HPC) that are committed to antigen-specific memory CD8⁺ cytotoxic T lymphocytes (CTL).

In some examples, iPSCs that form HPCs committed to antigen-specific memory CD8⁺CTLs may have higher expression levels of some genes as compared to those genes in CD3⁻ lymphocytes and/or non-lymphocytes. In some examples, iPSCs that form HPCs committed to antigen-specific memory CD8⁺CTLs may have lower expression levels of some genes as compared to those genes in CD3⁻ lymphocytes and/or non-lymphocytes.

Also disclosed are antigen-specific memory CD8+CTL generated from an iPSC. In some examples, the cells may be TAA-specific memory CD8+CTL. In some examples, the cells may be BCMA-specific CD8+CTL. In some examples, the CD8+CTL may be specific for the heteroclitic immunogenic BCMA₇₂₋₈₀ peptide (YLMFLLRKI (SEQ ID NO: 1)) and are re-differentiated from iPSC that form HPC committed to BCMA₇₂-80-specific CD8⁺ cytotoxic T lymphocytes (CTL).

The CTL obtained from the iPSC may be termed rejuvenated CTL. In some examples, the rejuvenated cells may be CD45RO+ memory cells (central memory and effector memory cells) and may have high expression of T cell activation (CD38, CD69) and/or costimulatory (CD28, CD40L, OX40, GITR) molecules. These cells may not have inhibitory receptors (CTLA4, PD1, LAG3, Tim3) or immune suppressive cells. In some examples, the rejuvenated CTL are functionally rejuvenated, have longer telomeres than the original CTL from which the iPSC was derived, and/or have higher proliferative potential than the original CTL from which the iPSC were derived.

In some examples, the cells may have a specific response against tumor cells. In some examples, the rejuvenated CTL may have a specific response to multiple myeloma cells with CD3⁺CD8⁺CTL proliferation in antigen-specific and HLA-A2-restricted manners.

In some embodiments, the cells disclosed herein may be used therapeutically in a patient or subject. In some embodiments, iPSCs, CD8T+ T cells, or combinations thereof may be used therapeutically in a patient or subject. In some embodiments, the cells may be used to treat proliferative diseases or disorders in the patient. In some embodiments, the proliferative disorders may be various cancers which may be metastatic or nonmetastatic. In some embodiments, the cancer may include multiple myeloma.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Herein, “antigen-specific” refers to specificity of some cells to recognize a specific antigen.

Herein, “B-cell maturation antigen” or “BCMA”, also called tumor necrosis factor receptor superfamily member 17 (TNFRS17) is a type III transmembrane protein that is generally expressed in malignant plasma cells, including multiple myeloma cells.

Herein, “cytotoxic T lymphocyte” or “CTL” (or CD8+ T cell) refers to T cells that can kill certain other cells.

Herein, “downregulated” refers to reduced expression of a gene product (e.g., mRNA, protein) in a cell.

Herein, “genetically modified” refers to cells into which various genes have been inserted. In some examples, genetically modified refers to cells reprogrammed to iPSCs by the genes encoding reprogramming factors.

Herein, “hematopoietic progenitor cell” or “HPC” refers to cells that develop from hematopoietic stem cells (HSCs) that can divide and further differentiate.

Herein, “immune cell” refers to cells that are part of the immune system.

Herein, “induced pluripotent stem cell” or “iPSC” refers to cells that have been reprogrammed to an embryonic-like, pluripotent state. iPSCs are generally capable of re-differentiating into other cell types.

Herein, “iPSC [CD3⁻ lymphocytes]” refers to iPSC cells that produce hematopoietic progenitor cells (HPC) committed to forming lymphocytes that do not express CD3 (therefore, they are CD3 negative or CD3⁻), like B cells, NK cells or NKT cells.

Herein, “iPSC [CD8⁺ T cells]” refers to iPSC cells that produce hematopoietic progenitor cells (HPC) committed to forming CD8⁺ T cells (e.g., CD8⁺CTL).

Herein, “iPSC [non-lymphocytes]” refers to iPSC cells that produce hematopoietic progenitor cells (HPC) committed to forming non-lymphocytes. These cells include, for example, monocytes and granulocytes.

Herein, “multiple myeloma” refers to abnormal plasma cells that proliferate and form tumors in the bones.

Herein, “re-differentiate” refers to the process of an iPSC becoming a differentiated cell.

Herein, “rejuvenated” refers to cells that are, in some aspects, physiologically younger than the cells from which they were derived (e.g., reset of telomere length, gene expression, oxidative stress, mitochondrial metabolism, and the like).

Herein, “re-program” refers to the process of a somatic cell becoming an iPSC.

The term “sample” can refer to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; blood mononuclear cells; serum; plasma; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Embodiments as described herein can involve isolating, collecting, or obtaining a biological sample from a subject. As used herein, the term “collecting a sample” or “isolating a sample”, for example, can refer to any process for directly or indirectly acquiring a biological sample from a subject. For example, a biological sample may be obtained (e.g., at a point-of-care facility, e.g., a physician's office, a hospital, laboratory facility) by procuring a tissue sample (such as a skin biopsy) from a subject. Alternatively, a biological sample may be obtained by receiving the biological sample (e.g., at a laboratory facility) from one or more persons who procured the sample directly from the subject. The biological sample may be, for example, a tissue (e.g., biopsy), fluid (e.g., cerebrospinal fluid, plasma, blood, serum) or cell (e.g., skin fibroblast cells, peripheral blood cells) of a subject.

The term “subject” or “patient” can refer to any organism to which aspects of the invention can be performed, e.g., for experimental, diagnostic, prophylactic, and/or therapeutic purposes. Subjects to which methods as described herein are performed comprise mammals, such as primates, for example humans. For veterinary applications, a wide variety of subjects are suitable, e.g., livestock such as cattle, sheep, goats, cows, swine, and the like; poultry such as chickens, ducks, geese, turkeys, and the like; and domesticated animals and pets such as dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject. The term “normal subject” can refer to a subject that is not afflicted with a disease or condition, such as a subject that is not afflicted with a cancer.

The term “therapeutically effective amount”, as used herein, can refer to an amount of a therapeutic agent whose administration, when viewed in a relevant population, correlates with or is reasonably expected to correlate with achievement of a particular therapeutic effect. The therapeutic effect may be objective (i.e., measurable by some test or marker) or subjective (i.e., subject gives an indication of or feels an effect). In some embodiments, a therapeutically effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. Disease progression can be monitored by clinical observations, laboratory and imaging investigations apparent to a person skilled in the art. A therapeutically effective amount is administered in a dosing regimen that can comprise multiple unit doses. For a therapeutic agent, a therapeutically effective amount (and/or an appropriate unit dose within an effective dosing regimen) can vary, for example, depending on route of administration, on combination with other pharmaceutical agents. Also, the specific therapeutically effective amount (and/or unit dose) for a patient can depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts. Furthermore, an effective amount may be administered via a single dose or via multiple doses within a treatment regimen. In some embodiments, individual doses or compositions are considered to contain a “therapeutically effective amount” when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be therapeutically effective as described herein.

The word “treating” can refer to the medical management of a subject, e.g., an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to a patient. For example, “treating” a cell proliferation disease, such as cancer, can refer to (or be indicated by) reduced levels of “M” proteins in the blood and urine. The “M” proteins are the abnormal monoclonal antibodies that are produced by the myeloma plasma cells. Plasma cells are derived from antibody-producing B cell lymphocytes; in the case of myeloma plasma cells, there is an overgrowth of the monoclonal antibodies collectively known as “M” proteins. There are several tests that are used to diagnose multiple myeloma, but production of the “M” proteins is central to the diagnosis. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

Herein, “tumor antigen” refers to antigens on tumor cells. Generally, tumor antigens are expressed at higher levels in tumor cells than in non-tumor cells (e.g., tumor-associated antigens or TAA). In some examples, tumor antigens can be expressed in certain tumor cells and not in non-tumor cells (e.g., tumor-specific antigens or TSA).

Herein, “tumor-associated antigen” or “TAA” refers to antigens that have elevated levels on tumor cells compared to normal cells. Generally, TAA can be expressed on normal cells. “Tumor-specific antigen” refers to antigens present on tumor cells and not on normal cells.

Herein, “upregulated” refers to increased expression of a gene product (e.g., mRNA, protein) in or on a cell.

As used herein, the phrase “therapeutic agent” can refer to any agent that elicits a desired pharmacological effect when administered to a subject. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, delay onset of, reduce severity of, and/or reduce incidence of one or more symptoms or features of a disease, disorder, and/or condition.

Induced Pluripotent Stem Cells (iPSO and Re-Differentiation of iPSCs

iPSCs are generally produced from differentiated, somatic cells by expression of reprograming factors. Re-programming factors can include genes or gene products from c-MYC, OCT3/4, SOX2 and KLF4 genes. In some examples, OCT3/4, SOX2 and KLF4, plus other optional factors, can be used. In some examples, vectors can be used to introduce genes encoding re-programming factors into the somatic cells.

Generally, the cells re-programmed to iPSCs can be any type of cell. In some examples, the cells may be human cells. In some examples, blood or skin cells may be used. In some examples, immune cells may be used. Generally, the immune cells may be of any type and can be lymphocytes or non-lymphocytes. In some examples, the cells may be B cells or T cells. In some examples, the immune cells may be specific for an antigen. In some examples, the T cells may be CD8+ or CTL cells, CD4+ or helper cells, or regulatory T cells (T_{reg}). In some examples, the the CTL cells may be specific for a specific antigen. In some examples, the CTLs may be specific for tumor antigens, including tumor-associated antigens (TAA) or tumor-specific antigens (TSA). In some examples, the CTLs may be specific for B cell maturation antigen (BCMA). In some examples, IFN-γ producing, BCMA-specific CTL were generated in vivo and used for the re-programming. In some examples, the generated cells may have increased expression of T cell activation markers (e.g., CD69⁺, CD38⁺) and/or co-stimulatory markers (e.g., CD40L⁺, OX30⁺, GITR⁺, 41BB⁺).

The iPSCs produced in the re-programming generally are able to proliferate, have a normal karyotype, may express stem cell markers like SSEA-4 and/or TRA-1-60, may differentiate into ectoderm, mesoderm and endoderm, and/or may retain alkaline phosphate during colony formation. The iPSCs may be capable of indergoing embroid body formation.

In some embodiments, hematopoietic progenitor cells (HPCs) may be isolated from embryoid bodies formed from the iPSCs. In some embodiments, the HPCs may be CD34⁺CD43⁺/CD14⁻ CD235a⁻. Some of the cells could re-differentate into CD3⁺ TCRαβ⁺/CD45⁺ T cells.

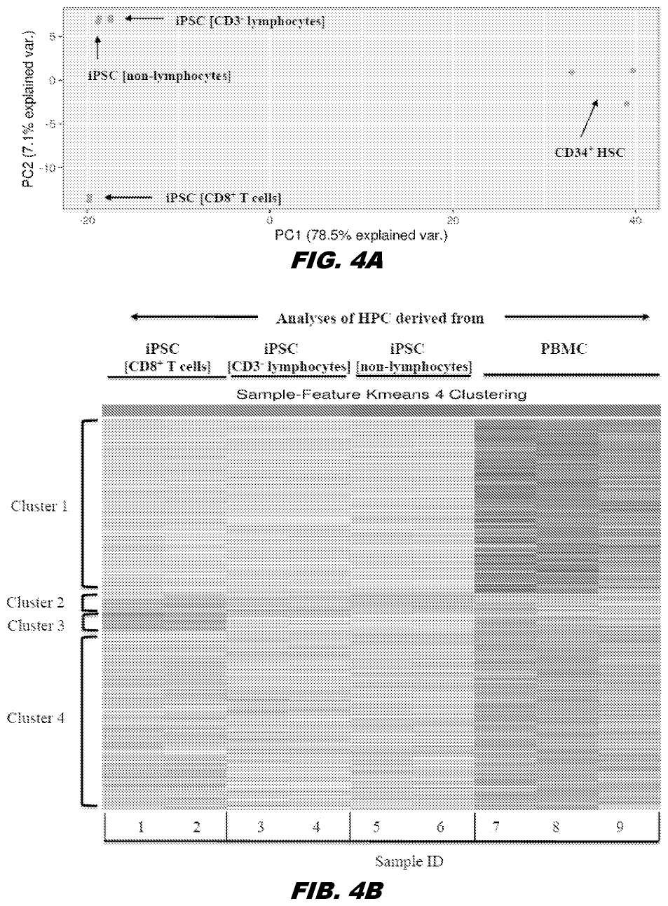

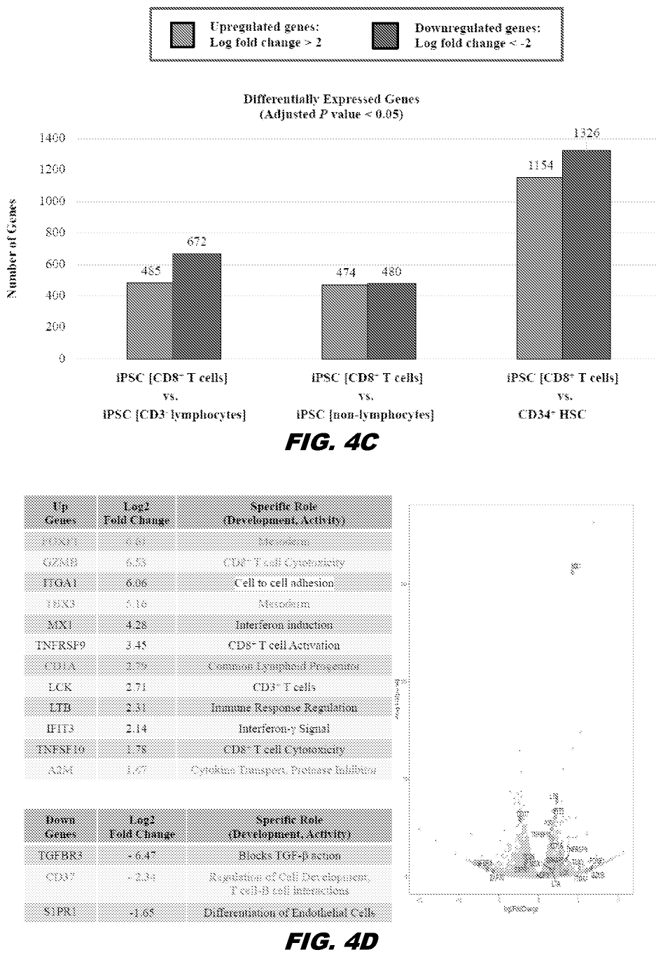

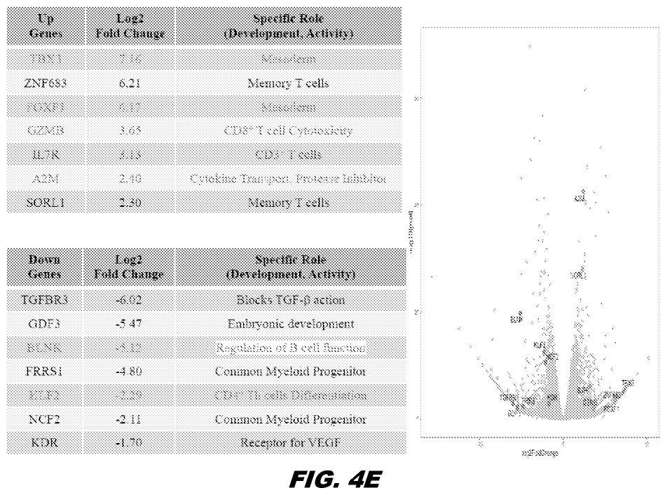

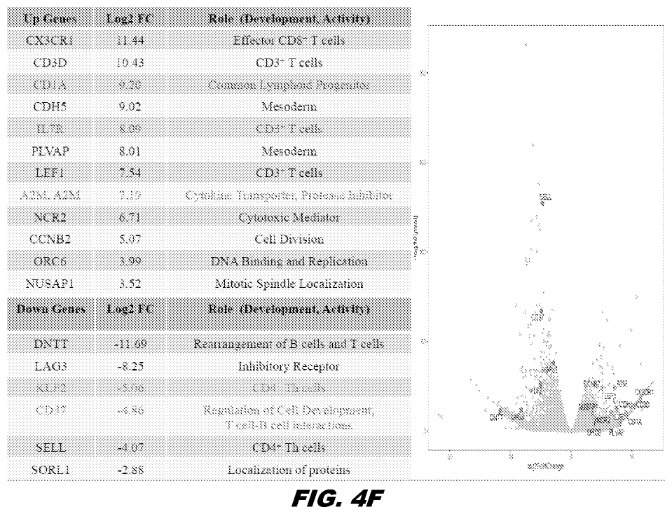

In some embodiments, CD34⁺CD43⁺/CD14⁻ CD235a⁻ HPC from iPSC clones may be committed to various re-differentiation pathways. In some embodiments, the HPC may be committed to CD8⁺ T cells (e.g., CD8⁺CTL) and may be called iPSC [CD8⁺ T cells]. In some examples, the HPC may be committed to CD3⁻ lymphocytes and may be called iPSC [CD3⁻ lymphocytes]. iPSC [CD3⁻ lymphocytes] may re-differentiate to B cells, NK cells and/or NKT cells, for example. In some embodiments, the HPC may be committed to to non-lymphocytes and may be called iPSC [non-lymphocytes]. iPSC [non-lymphocytes] may re-differentiate to monocytes and/or granulocytes, for example. Differences in gene expression in the HPCs committed to different pathways have been found (FIGS. 4B-4F).

In some embodiments, iPSC [CD8⁺ T cells] have increased expression of one or more genes as compared to iPSC [CD3⁻ lymphocytes]. In some embodiments, iPSC [CD8⁺ T cells] can have increased expression of 1, 2, 3, 4, 5, 6, 7, 8, 9, 10, 11 or all of genes FOXF1, GZMB, ITGA1, TBX3, MX1, TNFRSF9, CD1A, LCK, LTB, IFIT3, TNFSF10 and A2M as compared to iPSC [CD3⁻ lymphocytes].

In some embodiments, iPSC [CD8⁺ T cells] have decreased expression of one or more genes as compared to iPSC [CD3⁻ lymphocytes]. In some embodiments, iPSC [CD8⁺ T cells] can have decreased expression of 1, 2 or all of genes TGFBR3, CD37 and SlPRl as compared to iPSC [CD3⁻ lymphocytes].

In some embodiments, iPSC [CD8⁺ T cells] have increased expression of one or more genes as compared to iPSC [non-lymphocyte]. In some embodiments, iPSC [CD8⁺ T cells] can have increased expression of 1, 2, 3, 4, 5, 6 or all of genes TBX3, ZNF683, FOXF1, GZMB, IL7R, A2M and SORL1 as compared to iPSC [non-lymphocyte].

In some embodiments, iPSC [CD8⁺ T cells] have decreased expression of one or more genes as compared to iPSC [non-lymphocyte]. In some embodiments, iPSC [CD8⁺ T cells] can have decreased expression of 1, 2, 3, 4, 5, 6 or all of genes TGFBR3, GDF3, BLNK, FRRS1, KLF2, NCF2 and KDR as compared to iPSC [non-lymphocyte].

In some embodiments, iPSC [CD8⁺ T cells] have increased expression of one or more genes as compared to CD34⁺CD43⁺/CD14⁻ CD235a hematopoietic progenitor cells (HPC) derived from the iPSC [CD8⁺ T cells]. In some embodiments, iPSC [CD8⁺ T cells] have increased expression of 1, 2, 3, 4, 5, 6, 7, 8, 9, 10, 11 or all of genes CX3CR1, CD3D, CD1A, CDH5, ILR7, PLVAP, LEF1, A2M, NCR2, CCNB2, ORC6 and NUSAP1 as compared to HPCs derived from the iPSC [CD8⁺ T cells].

In some embodiments, iPSC [CD8⁺ T cells] have decreased expression of one or more genes as compared to CD34⁺CD43⁺/CD14⁻ CD235a hematopoietic progenitor cells (HPC) derived from the iPSC [CD8⁺ T cells]. In some embodiments, iPSC [CD8⁺ T cells] have decreased expression of 1, 2, 3, 4, 5 or all of genes DNTT, LAG3, KLF2, CD37, SELL and SORL1 as compared to HPCs derived from the iPSC [CD8⁺ T cells].

In some embodiments, iPSC [CD8⁺ T cell] has increased expression of one or more genes as compared to iPSC [CD3⁻ lymphocyte] and iPSC [non-lymphocyte]. In some embodiments, iPSC [CD8⁺ T cell] has increased expression of 1, 2, 3, 4, 5, 6, 7, 8, 9 or all genes TBX3, HOXA11, IRF4, PIK3C2B, KLF15, IL-12B, MAPK4, ITLN 1/2, TRIM6, EDA2R genes as compared to iPSC [CD3⁻ lymphocyte] and iPSC [non-lymphocyte].

In some embodiments, iPSC [CD8⁺ T cell] has decreased expression of one or more genes as compared to iPSC [CD3-lymphocyte] and iPSC [non-lymphocyte]. In some embodiments, iPSC [CD8⁺ T cell] has decreased expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or all genes RPS6KA2, CDK3, YEPL4, BATF2, BTN3A1, BTN3A1, USP44, CD70, ZXDA, FGFR1, NPM2, GGN, SPAG1, CATSPER2, N4BP3, P2RY14, NLGN2, SHC2, GRASP, AMIGO2, TBC1D32, CACNA1A, SLC6A9, HEYL, NEURL, RAB39B, ANK1, PSD, LRRK1, RUNX2, CXCL5, SEMA7A, JDP2, PLA2G6, MAP3K9, PIPOX, TNFRSF6B genes as compared to iPSC [CD3⁻ lymphocyte] and iPSC [non-lymphocyte].

In some examples, rejuvenated CD8+ T cells (e.g., CD8⁺CTL) are re-differentiated from iPSCs. In some examples, the rejuvenated CD8+ T cells (e.g., CD8⁺CTL) may have longer telomeres, higher proliferative potential, and the like, as compared to the cells from with the iPSCs were re-programmed.

Generally, the T cells are highly proliferative to target cells expressing an antigen to which the T cells have specificity. Generally, the specificity may be MHC-restricted. The T cells may have antigen-specific activity against tumor cells, (e.g., multiple myeloma cells).

The T cells re-differentiated from the iPSCs may be memory cells (e.g., CD45RO⁺). The memory calls may be central memory cells (CCR7⁺CD45RO⁺) or effector memory cells (CCR7⁻ CD45RO⁺), for example.

### Antigens

Herein, the cells that are reprogrammed to iPSCs (e.g., CTLs), the iPSCs, and the CTLs that are re-differentiated from the iPSCs may be specific for an antigen. Generally, any antigen may be used to produce these cells.

In some embodiments, these antigens may be cellular antigens or antigens from infectious or pathogenic agents. Cellular antigens, for example, may be displayed on the cell surface, may be located intracellulary, or both. In some examples, these antigens may include tumor antigens, tumor-associated antigens, tumor-specific antigens and the like. In some examples, these antigens may include products of mutated oncogenes or tumor suppressor genes, cellular proteins that are aberrant or overexpressed, antigens produced by oncogenic viruses, oncofetal antigens, cell surface glycoproteins or glycolipids, differentiation antigens specific to certain cell types, and the like.

Examples of these proteins (but not an inclusive list) may include normal, mutant or aberrant antigens: alphafetoprotein (AFP), carcinoembryonic antigen (CEA), CA-125, MUC-1, epithelial tumor antigen (ETA), p53, tyrosinase, melanoma-associated antigen (MAGE), ras.

In some embodiments, the antigen may be a putative target for multiple myeloma calls. In some embodiments, the antigen can be BCMA, NY-ESO-1, BCMA/CD19 or BCMA/CD38, CD4, CD22, CD44, CD138, GPRC5D, HA-1, SLAM7, TnMUC1, and others.

In some embodiments, the antigen may include B-cell maturation antigen (BCMA), also called tumor necrosis factor receptor superfamily member 17 (TNFRSF17). BCMA is generally expressed on mature B cells. BCMA may be associated with leukemias, lymphomas and multiple myeloma. In some examples, the antigen may be heteroclitic immunogenic BCMA₇₂₋₈₀ peptide (YLMFLLRKI (SEQ ID NO: 1)).

### Therapeutic Preparations

Aspects of the invention are drawn towards therapeutic preparations. As used herein, the term “therapeutic preparation” can refer to any compound or composition (e.g., including cells) that can be used or administered for therapeutic effects. As used herein, the term “therapeutic effects” can refer to effects sufficient to result in amelioration of symptoms, e.g., treatment, healing, prevention or amelioration of the relevant medical condition, or an increase in rate of treatment, healing, prevention or amelioration of such conditions. In some embodiments, therapeutic effect may refer to those resulting from treatment of cancer in a subject of patient.

Embodiments as described herein can be administered to a subject in the form of a pharmaceutical composition or therapeutic preparation prepared for the intended route of administration. Such compositions and preparations can comprise, for example, the active ingredient(s) and a pharmaceutically acceptable carrier. Such compositions and preparations can be in a form adapted to oral, subcutaneous, parenteral (such as, intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions and preparations can appear in conventional forms, for example, solutions and suspensions for injection, capsules and tablets, in the form of enteric formulations, e.g., as disclosed in U.S. Pat. No. 5,350,741, and for oral administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N. J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In embodiments, administering can comprise the placement of a pharmaceutical composition into a subject by a method or route which results in at least partial localization of the composition at a desired site such that desired effect is produced.

For example, the pharmaceutical composition can be administered by bolus injection or by infusion. A bolus injection can refer to a route of administration in which a syrine is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. Administration can continue for up to one year. In embodments, administration can continue for the life of the subject. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration in a continuous manner, such as to maintain the therapeutic effect (activity) over a prolonged period of time.

The pharmaceutical or therapeutic carrier or diluent employed can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier will vary widely but will usually be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition and/or preparation can also be in a form suited for local or systemic injection or infusion and can, as such, be formulated with sterile water or an isotonic saline or glucose solution. The compositions can be in a form adapted for peripheral administration only, with the exception of centrally administrable forms. The compositions and/or preparations can be in a form adapted for central administration.

The compositions and/or preparations can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The compositions and/or preparations can contain pharmaceutically and/or therapeutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

### Treating Disease, Disorder, Condition

Embodiments are also drawn towards methods of treating a disease, disorder, or condition such as a cell proliferative disease or disorder. A “cell proliferative disease or disorder” or can refer to a disease or disorder wherein unwanted cell proliferation of one or more subset(s) of cells in a multicellular organism occurs, resulting in harm to the multicellular organism. For example, the cell proliferative disease or disorder is cancer.

The terms “cancer” and “cancerous” can refer to or describe the physiological condition in mammals that is characterized by unregulated cell growth. Examples of cancer include, but are not limited to, blood-borne cancers (e.g., multiple myeloma, lymphoma and leukemia), and solid cancers.

In embodiments, the cancer can comprise those that are metastatic or are not metastatic or are metastatic.

In embodiments, the cancer can include, but is not limited to, solid cancer and blood borne cancer.

Examples of cancers can include, but not be limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastasis, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4 colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

The term “tumor” can refer to all neoplastic cell growth and proliferation, whether malignant or benign, and all pre-cancerous and cancerous cells and tissues. “Neoplastic,” as used herein, can refer to any form of dysregulated or unregulated cell growth, whether malignant or benign, resulting in abnormal tissue growth. Thus, “neoplastic cells” can include malignant and benign cells having dysregulated or unregulated cell growth.

“Blood borne cancer” or “hematologic malignancy” can refer to cancer of the body's blood-forming and immune system—the bone marrow and lymphatic tissue. Such cancers include leukemias, lymphomas (Non-Hodgkin's Lymphoma), Hodgkin's disease (also called Hodgkin's Lymphoma) and myeloma. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders, chronic myelogenous leukemia (CML), myelodysplastic syndrome (MDS), human lymphotropic virus-type 1 (HTLV-1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma.

The major types of cancer are carcinomas, sarcomas, melanomas, lymphoma, and leukemias. Carcinomas originate in the skin, lungs, breasts, pancreas, and other organs and glands. Lymphomas are cancers of lymphocytes. Leukemia is cancer of the blood. It does not usually form solid tumors. Sarcomas arise in bone, muscle, fat, blood vessels, cartilage, or other soft or connective tissues of the body. Melanomas are cancers that arise in the cells that make the pigment in skin. Non-limiting examples of cancers include ovarian cancer, breast cancer, lung cancer, prostate cancer, cervical cancer, pancreatic cancer, colon cancer, stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. In an embodiment, the cancer comprises one or more of a colon cancer, colorectal cancer, gastro-intestinal cancer, breast cancer, bladder cancer, kidney cancer, leukemia, brain cancer, sarcoma, astrocytoma, acute myelogenous leukemia (AML), and diffuse large B-lymphoma.

In embodiments, the cancer comprises multiple myeloma. Non-limiting examples of cancers that can be treated by embodiments described herein comprise multiple myeloma, kidney cancer, breast cancer, lung cancer, brain cancer, skin cancer, liver cancer, liposarcoma, and pancreatic cancer. These cancers can be treated using the embodiments described here, alone or in combination with other therapies used for these cancers.

The embodiments disclosed here can be used to treat myeloma or multiple myeloma. The embodiments disclosed here can also be used to treat precancerous or premalignant conditions. In some embodiments, the precancerous/premalignant conditions can be related to myeloma or multiple myeloma. In some embodiments, smoldering myeloma or smoldering multiple myeloma can be treated using the disclosures herein. In some embodiments, monoclonal gannopathy of undetermined significance can be treated using the disclosures herein.

### Kits

Aspects of the invention comprise a component a kit useful for treating or diagnosing a subject with a disease or disorder such as a cell proliferative disease or disorder.

In some examples, kits may include iPSCs, cells re-differentiated from iPSCs (e.g., tumor-specific CTLs), and/or the cells from which the iPSCs are re-programmed.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Here we disclose heteroclitic immunogenic BCMA₇₂₋₈₀ peptide (YLMFLLRKI (SEQ ID NO: 1))-specific induced pluripotent stem cells (iPSC) and re-differentiated T cells from iPSC, which can be used for treatment of patients with multiple myeloma.

Antigen-specific cytotoxic T lymphocytes (CTL) against tumor-associated antigens provide an important immune-system defense against cancer. Adoptive T-cell therapy, the administration of a large number of ex vivo expanded activated antigen-specific CTL targeting tumor specificantigens, has shown promise for delivering anti-tumor activities with durable remissions in certain malignancies. Genetic engineering using T cell receptor (TCR) genes or chimeric antigen receptor T-cells (CAR-T) are powerful approaches to improve the specificity and cytotoxicity of T cell therapies. One advantage of utilizing a TCR-based therapeutic is the ability to recognize intracellular antigens that have been processed and presented as immunogenic peptide complexes within MHC molecules (Johnson et al. 2009; Morgan et al. 2006). However, when exogenous TCR genes are introduced, mis-pairing of the transferred α and β chains with endogenous α and β chains can occur, with serious autoimmune adverse events reported in clinical trials (Bendle et al. 2010, Hinrichs et al. 2013). In contrast, CAR-T cells recognize antigens expressed on the cell surface in a non-MHC-restricted manner. One successful CAR-T therapy comprises targeting the B-cell marker antigen CD19, which has demonstrated the induction of complete remission even in patients with relapsed and chemo-refractory B-cell malignancies (Kochenderfer et al. 2010, Grupp et al. 2013). However, off-tumor toxicity including cytokine release syndrome is a problem of this therapeutic approach and the safety and effectiveness of the therapy are still being examined in clinical trials (Brentjens et al. 2011, Kalos et al. 2011, Kochenderfer et al. 2012, Porter et al. 2011). Importantly, endogenous and ex vivo expanded and administered cancer patients' CTL are continuously exposed to tumor antigens with long-term expansion, can become unable to proliferate (also referred to as “exhausted”) and can lose their functional activities with terminal differentiation. For most cancers, the loss of CTL immune functional responses limits their clinical utility.

Without being bound by theory, embodiments of the compositions and methods disclosed herein exploit rejuvenated iPSC-derived antigen-specific CTL technology as an adoptive T-cell therapeutic strategy targeting multiple myeloma. In one embodiment, the selective reprograming of ex vivo generated BCMA antigen-specific CTL clones was performed to orient them into rejuvenated antigen-specific T cells by re-differentiating T cells from the iPSC (T-iPSC) to increase their ability for self-renewal and maintain enhanced long-term cytotoxicity against tumor cells. Results indicate that BCMA-specific T-iPSC have the capacity to differentiate into CD8αβ T cells from BCMA-iPSC, which supports the use of iPSC as a cell source for producing CD8⁺CTL with the advantages in the antigen specificity, rejuvenation profile, reproducible number of CTL, or a combination thereof. Without being boud by theory, the compositions and methods disclosed herein permit therapeutically applicable regenerative T cell immunotherapies that effectively treat the patients with myeloma.

Exploitation of fully rejuvenated CTL from induced pluripotent stem cells (iPSC) can be used to overcome CTL exhaustion and muted functional anti-tumor responses (Good et al. 2019, Minagawa et al. 2018, Ando et al. 2016). In embodiments, these iPSC comprise a special type of pluripotent cells that are derived from adult somatic cells upon ectopic expression of a defined set of transcription factors. Notably, tumor antigen-specific CTL can be reprogrammed with iPSC technology from the original antigen-specific CTL (Vizcardo et al. 2013, Ando et al. 2015, Timmermans et al. 2009, Kennedy et al. 2012). These iPSC-CTL are functionally rejuvenated, demonstrate longer telomeres and have a higher proliferative capacity (5-50 fold increase) than their original CTL. This approach has been improved in last several years, and induced CD8αβ-expressing iPSC-T cells, like physiological CTL, show a higher proliferation and antigen-specific cytotoxicity than CD8αα expressing ones, like innate immune cells. In addition, an approach to differentiate T-cells from iPSCs without a support of stroma cells and exogeneous serum has been developed for clinical application (Themeli et al. 2013, Sturgeon et al. 2014, Huijskens et al. 2014). Thus, this reprogramming therapeutic approach has the potential to increase the efficacy of other cellular antigen-specific cancer immunotherapies.

In the present disclosure, we generate and develope iPSC specific to BCMA, the receptor for binding of B cell activating factor (BAFF) and a proliferation-inducing ligand (APRIL), important in promoting MM cell growth and survival, by generating the antigen-specific memory CD8⁺CTL and induce effective and long-lasting immune response against tumor cells. These BCMA-specific iPSC can be established using an engineered peptide specific to BCMA, BCMA₇₂₋₈₀ (YLMFLLRKI (SEQ ID NO: 1)), which display improved affinity/stability to HLA-A2 from their native peptides and evoke BCMA-specific CTL displaying increased activation (CD38, CD69) and co-stimulatory (CD40L, OX40, GITR) molecule expression. Especially, the heteroclitic BCMA₇₂₋₈₀ CTL demonstrated the polyfunctional Th1-specific activities [IFN-γ/IL-2/TNF-α production, proliferation, cytotoxicity] against MM, which were directly correlated with expansion of Tetramer+ and memory CD8⁺CTL population. When combined with anti-OX40 or anti-LAG3, heteroclitic BCMA₇₂₋₈₀ CTL displayed increased cytotoxicy against MM by central memory CTL. Thus, these results provide the framework for therapeutic application of heteroclitic BCMA peptides to generate iPSC-T cells as adoptive T cells immunotherapy as a single therapy or combination treatment options incorporating the vaccine specific to BCMA or other antigens.

### REFERENCES CITED IN THIS EXAMPLE

L. A. Johnson, R. A. Morgan, M. E. Dudley, et al. Gene therapy with human and mouse T-cell receptors mediates cancer regression and targets normal tissues expressing cognate antigen. Blood, 114 (2009), pp. 535-546.
R. A. Morgan, M. E. Dudley, J. R. Wunderlich, et al. Cancer regression in patients after transfer of genetically engineered lymphocytes. Science, 314 (2006), pp. 126-129.
G. M. Bendle, C. Linnemann, A. I. Hooijkaas, et al. Lethal graft versus-host disease in mouse models off cell receptor gene therapy. Nat Med, 16 (2010), pp. 565-570.
C. S. Hinrichs, N. P. Restifo. Reassessing target antigens for adoptive T cell therapy. Nat Biotechnol, 31 (2013), pp. 999-1008.
J. N. Kochenderfer, W. H. Wilson, J. E. Janik, et al. Eradication of B-lineage cells and regression of lymphoma in a patient treated with autologous T cells genetically engineered to recognize CD19. Blood, 116 (2010), pp. 4099-4102.
S. A. Grupp, M. Katos, D. Barrett, et al. Chimeric antigen receptor modified T cells for acute lymphoid leukemia. N Engl J Med, 368 (2013), pp. 1509-1518.
R. J. Brentjens, I. Riviere, J. H. Park, et al. Safety and persistence of adoptively transferred autologous CD 19-targeted T cells in patients with relapsed or chemotherapy refractory B-cell leukemias. Blood, 118 (2011), pp. 4817-4828.
M. Kalos, B. L. Levine, D. L. Porter, et al. T cells with chimeric antigen receptors have potent antitumor effects and can establish memory in patients with advanced leukemia. Sci Transl Med, 3 (2011), p. 95ra73.
J. N. Kochenderfer, M. E. Dudley, S. A. Feldman, et al. B-cell depletion and remissions of malignancy along with cytokine-associated toxicity in a clinical trial of anti-CD19 chimericantigen-receptor-transduced T cells. Blood, 119 (2012), pp. 2709-2720.
D. L. Porter, B. L. Levine, M. Katos, A. Bagg, C. H. JuneChimeric antigen receptor-modified T cells in chronic lymphoid leukemia. N Engl J Med, 365 (2011), pp. 725-733.
Good M L, Vizcardo R, Maeda T, Tamaoki N, Malekzadeh P, Kawamoto H, Restifo N P. J Vis Exp. 2019 Oct. 24; (152). Using Human Induced Pluripotent Stem Cells for the Generation of Tumor Antigen-specific T Cells.
Minagawa A, Yoshikawa T, Y asukawa M, Hotta A, Kunitomo M, Iriguchi S, Takiguchi M, Kassai Y, Imai E, Yasui Y, Kawai Y, Zhang R, Uemura Y, Miyoshi H, Nakanishi M, Watanabe A, Hayashi A, Kawana K, Fujii T, Nakatsura T, Kaneko S.
Enhancing T Cell Receptor Stability in Rejuvenated iPSC-Derived T Cells Improves Their Use in Cancer Immunotherapy. Cell Stem Cell. 2018 Dec. 6; 23(6):850-858.e4.
Ando M, Nishimura T, Yamazaki S, Yamaguchi T, Kawana Tachikawa A, Hayama T, Nakauchi Y, Ando J, Ota Y, Takahashi S, Nishimura K, Ohtaka M, Nakanishi M, Miles J J, Burrows S R, Brenner M K, Nakauchi H. A Safeguard System for Induced Pluripotent Stem CellDerived Rejuvenated T Cell Therapy. Stem Cell Reports. 2015 Oct. 13; 5(4):597-608.
R. Vizcardo, K. Masuda, D. Yamada, et al. Regeneration of human tumor antigen-specific T cells from iPSCs derived from mature CDS(+) T cells. Cell Stem Cell, 12 (2013), pp. 31-36.
M. Ando, T. Nishimura, S. Yamauki, et al. A safeguard system for induced pluripotent stem cellderived rejuvenated T-cell therapy. Stem Cell Rep, 5 (2015), pp. 597-608.
F. Timmermans, I. Velghe, L. Vanwalleghem, et al. Generation of T cells from human embryonic stem cell-derived hematopoietic zones. J Immunol, 182 (2009), pp. 6879-6888.
M. Kennedy, G. Awong, C. M. Sturgeon, et al. T lymphocyte potential marks the emergence of definitive hematopoietic progenitors in human pluripotent stem cell differentiation cultures. Cell Rep, 2(2012), pp. 1722-1735.
T. Nishimura, S. Kaneko, A. Kawana-Tachikawa, et al. Generation of rejuvenated antigen-specific T cells by reprogramming to pluripotency and redifferentiation. Cell Stem Cell, 12 (2013), pp. 114-126.
Themeli M, Kloss C C, Ciriello G, Fedorov V D, Perna F, Gonen M, Sadelain M. Generation of tumor-targeted human T lymphocytes from induced pluripotent stem cells for cancer therapy. Nat Biotechnol. 2013 October; 3 1(10):928-33.
Sturgeon C M, Ditadi A, Awong G, Kennedy M, Keller G. Wnt signaling controls the specification of definitive and primitive hematopoiesis from human pluripotent stem cells. Nat Biotechnol. 2014 June; 32(6):554-61.
Huijskens M J, Walczak M, Koller N, Briede J J, Senden-Gijsbers B L, Schnijderberg M C, Bos G M, Germeraad W T. Technical advance: ascorbic acid induces development of double-positive T cells from human hematopoietic stem cells in the absence of stromal cells. J Leukoc Biol. 2014 December; 96(6):1165-75.

### Example 2

### Abstract

T cell regenerative medicine represents an emerging immunotherapeutic approach, especially by using antigen-specific Induced Pluripotent Stem Cells (iPSC) to rejuvenate CD8⁺ cytotoxic T lymphocytes (CTL). Here, we disclosean iPSC-derived therapeutic strategy targeting B-Cell Maturation Antigen (BCMA) against multiple myeloma (MM) via development of rejuvenated T cells possessing highly functional anti-tumor activities. Upon initial establishment of BCMA-specific iPSC characterized by key stem cell markers, pluripotency potential, and normal karyotypes, specific polarization was followed during embryoid body formation into mesoderm development, evidenced by specific activation of transcriptional regulators like SNAI2, TBX3, PLVAP, HAND1 and CDX2. RNAseq analyses identified specific transcriptional regulation pathways utilized by BCMA-specific iPSC clones during differentiation into CD8⁺CTL. The unique transcriptional profiles included upregulation of transcriptional regulators determining CD4/CD8 T cell differentiation ratio, memory CTL formation, NF-kappa-B/JNK pathway activation, and cytokine transporter/cytotoxic mediator development as well as downregulation of regulators controlling B and T cell interactions or CD4⁺ Th cells and inhibitory receptor development. In embodiments, the BCMA specific iPSC-T cells demonstrate (1) mature T cell phenotypes including central and effector memory CTL development without immune checkpoints expression, (2) a high proliferative (1,000×) capacity during T cell differentiation, (3) poly-functional anti-tumor activities and Th1-specific cytokine production to multiple myeloma in an antigen-specific and HLA-A2-restricted manner, (4) specific immune responses and CTL proliferation to cognate HLA-A2 heteroclitic BCMA₇₂₋₈₀ (YLMFLLRKI (SEQ ID NO: 1)) peptide and (5) distinct sole clonotype for T cell receptor. Furthermore, the specific iPSC clones maintained their differentiation potential into CD8⁺ memory T cells upon subcloning and long-term culture under feeder-free culture conditions. In conclusion, these results establish a framework for iPSC-based regenerative medicine to provide rejuvenated and highly functional memory CD8⁺BCMA-specific CTL as an adoptive immunotherapy to improve patient outcome in multiple myeloma.

Certain effective cancer therapy strategies aim to boost effector T cell development and function while abrogating mechanisms mediating immunosuppression in tumor microenvironment. As effector T cells, CD8⁺CTL have an importatnt role in protective immunity against cancer. However, constant exposure to antigens and various inflammatory signals within the tumor microenvironment leads to the T cell exhaustion and a loss of tumor antigen-specific functionality. Although remarkable responses have been demonstrated in CAR-T cell immunotherapy in some cancer patients, low responses or cancer relapse were reported in a significant number of patients, possibly by the loss of CAR target molecules on tumor cells and reduced in vivo persistence of transferred CAR-T cells due to T cell exhaustion and dysfunction through continuous T-cell receptor and cytokine stimulation.

Importantly, memory CD8⁺ CTL effectively respond to cognate tumor-associated antigens (TAA) with increased capacity to self-renew, which is important in establishing persistent long-term immunity, however they show a significant level of exhaustion in cancer patients as compared to other CD8⁺CTL populations, along with the development of various checkpoint molecules and immune suppressor cells. In parallel, sustained remission was associated with an elevated frequency of early memory CD8⁺CTL, before CAR-T cell generation for therapy. Thus, it is important to design a better therapeutic approach through optimization of T cell differentiation process for their fully functional anti-tumor activities, in order to improve the therapeutic efficacy of adoptive cell therapy in patients.

Here, we disclose a regenerative medicine approach upon establishment of BCMA-specific iPSC through defined epigenetic reprograming, which resulted to differentiate into highly functional rejuvenated memory CD8⁺CTL capable to respond to their cognate HLA-A2-specific antigen on multiple myeloma cells. We found that the selected iPSC clones utilize cell commitment pathways during transformation, distinctly into the antigen-specific CD8⁺CTL. We further identified specific exemplary transcriptional regulators (FOXF1, TBX3, GZMB, A2M; CD37, BLNK, KLF2) in the selected iPSC clones, which contribute to polarization into CD8⁺CTL and memory cells formation or block inhibitory receptors development, with an implication to utilize these transcriptional regulators on iPSC to specifically commit into the antigen-specific CD8⁺ CTL. The differentiated CD8⁺ CTL from the iPSC clones demonstrated high levels of proliferative and functional anti-tumor activities to multiple myeloma in the antigen-specific and HLA-A2 restricted manners, which were associated with the absence of checkpoint molecules expression and immune suppressive cells. We further detected that the anti-tumor activites were highly specific to their cognate HLA-A2 heteroclitic BCMA₇₂₋₈₀ (YLMFLLRKI (SEQ ID NO: 1)) peptide and occurred through a distinct sole T cell receptor clonotype on the resulted CD8⁺CTL differentiated.

In summary, we provide a study of regenerative T-cell immunotherapy, using the cellular reprogramming technology, which allow the generation of TAA-specific iPSC and commit to rejuvenated antigen-specific memory CTL, without further differentiation into terminal effector cells. These results provide evidence of benefits of BCMA-specific iPSC as a foundation to direct the rejuvenated T-cell immunotherapy to the clinic with self-renewal and pluripotency in order to accomplish a persistent long-term immunity in myeloma patients, which can be an exciting cellular therapeutic application to overcome the limitations seen in current adoptive T cells immunotherapies.

### INTRODUCTION

Adoptive T-cell therapy, as the administration of ex vivo expanded antigen-specific cytotoxic T lymphocytes (CTL) against tumor-associated antigens (TAA), provides an important immune defense against cancer and has shown an achieved durable remissions in selected malignancies (Chruściel et al. 2020, D'Ippolito et al. 2019). However, the patients' T cells in the tumor environment often lead the cells to be exhausted, leading them to be unable to respond nor maintain their poly-functional immune responses and terminal differentiation, which resulted in the non-accomplishment or loss of anti-tumor activities and clinical utility. Despite of advantage of CAR-T cells-based therapeutic approach with encouraging clinical outcome and complete remission in patients with relapsed and chemo-refractory malignancies, off-tumor toxicity including cytokine-release syndrome is a serious adverse event of this therapeutic approach, and both safety and effectiveness of the therapy are still being examined in clinical trials (Cappell et al. 2020, Halim et al. 2020, Shah et al. 2020, Andrea et al. 2020). Especially, CAR-T cells generated from cancer patients' T cells become further exhausted during or after the T cells manipulation and their ex vivo expansion. This supports a rationale that the reversal of T cell exhaustion and reprograming to early stage of memory T cells might be important to improve the efficacy of immunotherapies and their functional activities against tumor.

One technique to overcome T cells exhaustion and muted functional anti-tumor responses can comprise exploitation of fully rejuvenated CTL developed from iPSC (Nishimura et al. 2019, Good et al. 2019). T cell regenerative medicine can lead to rejuvenation of antigen-specific CD8+CTL and has a therapeutic potential to effectively treat patients with cancer. In an exemplary approach, mature somatic T cells can be reprogrammed to a pluripotent state through ectopic expression of key defined transcription factors, in a process known as induced pluripotency; the resultant iPSC exhibit transcriptional and epigenetic features and have the capacity of self-renewal and pluripotency, similarly to embryonic stem cells³⁻⁵, with the unlimited proliferative potential and ability to differentiate into any cell type. These pluripotent characteristics, coupled with the ability to derive iPSC from patient cells, have made iPSC a valuable tool for many diseases modeling and drug discovery.

Disclosed herein in one embodiment is a cellular reprogramming technology utilizing TAA-specific CD8+CTL, upon re-differentiation from the antigen-specific iPSC, which can be applied as a therapeutic application by merging of cancer immunotherapy with regenerative medicine. Here, we disclose an exemplary reprogramming approach of B-Cell Maturation Antigen (BCMA)-specific iPSC with key stem cell markers expression, pluripotency potential and normal karyotypes, and some of the iPSC clones can be committed to antigen-specific CD8+ CTL with mature type of T cell phenotype. The CD8+ T cells (e.g., CD8⁺CTL) differentiated from the BCMA-specific iPSC can be rejuvenated as CD45RO+ memory cells (central memory and effector memory cells) with high expression of T cell activation (CD38, CD69) and costimulatory (CD28) molecules, but without induction of inhibitory receptors (CTLA4, PD1, LAG3, Tim3) nor immune suppressive cells. Embodiments also demonstrated high induction of T cells proliferation and fully functional anti-tumor activities against multiple myeloma (MM), which include the specific responses to cognate HLA-A2 heteroclitic BCMA72-80 (YLMFLLRKI (SEQ ID NO: 1)) peptide and distinct display of sole clonotype for T cell receptor.

Upon our observation on the consistency and stability of the iPSC clone on polarization into the specific cell subset, one interest of the current disclsoure is to characterize the iPSC clones for their transcriptional regulation to commit into a specific cell subset. Without being bound by theory, a better understanding of the molecular mechanism of iPSC on the differentiation into specific cell subset along with rejuvenation will lead to clinical opportunities to treat patients. Here, we disclose our findings on the respective cell differentiation pathway of iPSC clones and their genetic mechanisms including regulatory elements, which play roles on specific cell commitment into CD8+CTL. This disclosure also reveals the exemplary transcriptional profiles of the iPSC, which polarize into each specific cell subset along with their respective genetic regulations on activation and repression sites, providing important information on how to orient and direct the iPSC to differentiate toward specific pathway, especially to generate into TAA-specific CD8+CTL as a therapeutic consideration.

In summary, the cellular technology disclosed in one embodiment herein allows for the establishment of antigen-specific iPSC via defined epigenetic reprograming with unique genomic landscapes. This approach can be beneficial for current clinical protocols and provides for self-renewal and pluripotency for the antigen-specific T cell therapies, and in one embodiment, applying the rejuvenated memory CD8+ T cells (e.g., CD8⁺CTL) with a high proliferative capacity and effective anti-tumor activities, thus increase therapeutic efficacy of cancer immunotherapy and effectively treat the patients with cancer. Without being bound by theory, certain embodiments herein comprise a therapeutic option to overcome the challenges in current cell therapy options, which induce exhaustion and terminal differentiation with poor cells survival, and thus provide the framework for therapeutic application in targeted immunotherapy to improve clinical outcome in MM patients.

### Methods Utilized in Embodiments of the Present Example

### Cell Lines

The MM cell lines, U266 (HLA-A2⁺BCMA⁺) and RPMI (HLA-A2⁻ BCMA⁺), and a breast cancer cell line MDA-MB-231 (HLA-A2⁺BCMA⁻) were obtained from ATCC (Manassas, VA). The T2 cell line, a human B and T cell hybrid expressing HLA-A2 molecules, was provided by Dr. J. Molldrem (University of Texas M. D. Anderson Cancer Center, Houston, TX). The K562 cell line transduced with HLA-A*0201 cDNA (K562-A*0201) was provided by Dr. P. Cresswell (Yale University). The cell lines were cultured in DMEM (for MM cells, T2 cells and K562-A*0201 cells; Gibco-Life Technologies, Rockville, MD) or Leibovitz's L-15 (for MDA-MB231; ATCC, Manassas, VA) media supplemented with 10% fetal calf serum (FCS; BioWhittaker, Walkersville, MD), 100 IU/ml penicillin and 100 μg/ml streptomycin (Gibco-Life Technologies).

### Reagents

Fluorochrome conjugated anti-human monoclonal antibody (mAb) specific to TRA-1-60, SSEA-4, BCMA, CCR7, CD3, CD4, CD5, CD7, CD8α, CD80, CD14, CD16, CD25, CD28, CD34, CD38, CD43, CD45, CD45RO, CD56, CD69, CD107a, CD235a, CTLA4, FoxP3, HLA-A2, PD1, LAG3, TIM3, TCRαβ, TCRγδ, IFN-γ, IL-2 or TNF-α was purchased from Becton Dickinson (BD) (San Diego, CA), LifeSpan Bioscience (Seattle, WA), BioLegend (San Diego, CA) or R&D Systems (Minneapolis, MN). Live/Dead Aqua stain kit was purchased from Molecular Probes (Grand Island, NY). Recombinant human GM-CSF was obtained from Immunex (Seattle, WA); and human IL-2, IL-4, IFN-α, and TNF-α were purchased from R&D Systems. Heteroclitic BCMA₇₂₋₈₀ (YLMFLLRKI (SEQ ID NO: 1)) peptide-specific Tetramer-P E was synthesized by MBL International Corporation (Woburn, MA).

### Synthetic Peptides

Heteroclitic BCMA₇₂₋₈₀ (YLMFLLRKI (SEQ ID NO: 1)) peptide and HIV-Gag₇₇₋₈₅ (SLYNTVATL (SEQ ID NO: 2)) were synthesized by standard fmoc (9-fluorenylmethyl-oxycarbonyl) chemistry, purified to >95% using reverse-phase chromatography, and validated by mass-spectrometry for molecular weight (Biosynthesis, Lewisville, TX).

Induction of Heteroclitic BCMA₇₂₋₈₀ (YLMFLLRKI (SEQ ID NO: 1)) Peptide-Specific CD8⁺CTL

The heteroclitic BCMA₇₂₋₈₀ (YLMFLLRKI (SEQ ID NO: 1))) peptide-specific CD8⁺CTL (BCMA-CTL) were generated ex vivo by repeated stimulation of enriched CD3⁺ T cells obtained from HLA-A2⁺ donors with APC pulsed with our previously reported highly immunogenic heteroclitic BCMA₇₂₋₈₀ (YLMFLLRKI (SEQ ID NO: 1)) peptide (Bae et al., 2019). In brief, the heteroclitic BCMA₇₂₋₈₀ peptide (50 μg/ml)-pulsed APC were irradiated (10 Gy) and used to stimulate CD3⁺ T cells at a 1 APC/peptide: 20 T cell ratio. Cultures were stimulated with peptide-pulsed APC every 7 days and maintained in AIM-V medium supplemented with 10% human AB serum (BioWhittaker, Walkersville, MD) in the presence of IL-2 (50 units/ml). After four rounds of stimulation, the BCMA-specific CTL were enriched for IFN-γ producing CD3⁺CD8⁺CTL by sorting on a FACS Aria II flow cytometer (Becton Dickinson (BD), San Jose, CA). The sorted cells were then utilized to establish BCMA-specific iPSC clones.

Establishment of iPSC from BCMA-CTL

FACS sorted IFN-γ producing heteroclitic BCMA₇₂₋₈₀ peptide-specific CTL were reprogrammed into stem cells using the CytoTune iPSC 2.0 Sendai Reprogramming Kit (Thermo Fisher Scientific, Waltham, MA), with minor modifications. Specifically, heteroclitic BCMA₇₂₋₈₀ peptide-specific IFN-γ producing CD8⁺CTL were transduced with reprogramming factors (OCT3/4, SOX2, KLF4, and c-MYC) via Sendai virus vectors at a MOI of 5 or 20. Additionally, an SV40LTAg-encoded vector (ID Pharma, Chiyoda-ku, Tokyo, Japan) was included during the process to enhance the reprogramming efficiency. The cells were cultured in DMEM media (Gibco-Life technologies, Rockville, MD) supplemented with 10% FBS (Gibco-Life technologies), 2 mM L-glutamine, 100 U/ml penicillin and 100 ng/ml streptomycin (Sigma Aldrich, St. Louis, MO), 10 ng/ml recombinant human IL-7 (Peprotech, Rocky Hill, NJ), and 10 ng/ml recombinant human IL-15 (R&D systems, Minneapolis, MN) during the reprograming process. Next, the reprogramed BCMA-specific CTL were transferred onto iMatrix-511 (Nippi, Adachi-ku, Tokyo, Japan)-coated 6-well plates (0.5 mg/cm²) and fed with StemFit Basic02 medium (Ajinomoto, Chuo-ku, Tokyo, Japan) containing 10 ng/ml recombinant human FGF-basic (Peprotech). Several number of iPSC colonies appeared within 21 days of culture and each of the colonies were picked, expanded and frozen down. Photomicrographs were taken of the individual iPSC clones with an inverted microscope (Carl Zeiss, Oberkochen, Germany). EBV-specific iPSC generated from EBV LMP2A peptide-specific CTL were provided by Dr. S. Kaneko at Kyoto University (Kyoto, Japan) and were used as a process control.

Development of BCMA-Specific iPSC or EBV-Specific iPSC Under Feeder-Free Cell Culture Conditions

BCMA- or EBV-specific iPSC clones were cultured under feeder-free culture conditions (Iriguchi et al., 2021) with some modifications. Reprogramed stem-like iPSC were cultured in iMatrix-511-coated culture plates and were passaged via dissociation into single cells using TrypLE Select (Life Technologies). The single-cell suspensions were re-plated into iMatrix-511-coated culture plate (1×10³ cells/cm²), along with 10 μM Rock inhibitor (Y-27632: R&D systems). The next day, the culture medium was switched to fresh StemFit Basic02 medium (Amsbio, Cambridge, MA) containing 10 ng/ml FGF-basic (R&D systems) and then changed every other day. Seven days after plating, the iPSC clones were collected and processed to undergo another round of passage.

Pluripotency Status of Reprogramed BCMA-Specific iPSC

BCMA-specific iPSC were evaluated for their pluripotency status. BCMA-specific iPSC colonies were collected 5 days after passage, stained to detect pluripotency markers with fluorochrome-conjugated human mAb specific to SSEA-4 (R&D systems) or TRA-1-60 (Beckton Dickinson), fixed in 2% paraformaldehyde, acquired on a LSRFortessa flow cytometer (Beckton Dickinson) and analyzed using FACS DIVA v8.0 (Beckton Dickinson) or FlowJo v10.0.7 (Tree star, Ashland, OR) software. The pluripotency status of BCMA-specific iPSC was further evaluated via their alkaline phosphatase activity using the iPSC colonies were fixed in 4% paraformaldehyde (Electron Microscopy Sciences, Hatfield, PA), washed in 0.1M Tris-HCl (pH 9.5), stained for alkaline phosphatase for 15 minutes at room temperature in the dark, washed with 0.1M Tris-HCl (pH 9.5) and resuspended in D-PBS; photomicrographs were then taken with an inverted microscope (Carl Zeiss).

### Three Germ Differentiation and Pluripotency Assay

Reprogrammed BCMA-specific iPSC were further evaluated for three-germ differentiation using the STEMdiff™ Trilineage Differentiation Kit (STEMCELL Technologies, Vancouver, BC, Canada). In brief, the cells were plated onto Matrigel (Corning) coated plates and treated with endoderm and mesoderm differentiation media for 5 days and ectoderm differentiation media for 7 days. The cells were harvested, permeabilized, stained with fluorochrome-conjugated human mAbs specific to SOX17 (R&D systems), Brachyury (R&D systems) or Pax-6 (BD), and analyzed using FACS DIVA v8.0 or FlowJo v10.0.7 (Tree star, Ashland, OR) software upon acquisition by a LSRFortessa flow cytometer.

Detection of Sendai Virus Residue in Reprogrammed BCMA-Specific iPSC

The status of transgene-free was evaluated in the reprogrammed BCMA-iPSC. Total RNA was extracted using a RNeasy micro kit (QIAGEN), complementary DNA was synthesized using a High-Capacity cDNA Reverse Transcription kit (Applied Biosystems), and RT-PCR was performed using ExTaq HS (Takara). The primer sequences “5′-GGA TCA CTA GGT GAT ATC GAG C-3′ (SEQ ID NO: 3) (Forward) and 5′-ACC AGA CAA GAG TTT AAG AGA TAT GTA TC-3′ (SEQ ID NO: 4) (Reverse)” for SeV or “5′-GAA GGT GAA GGT CGG AGT C-3′ (SEQ ID NO: 5) (forward) and 5′-GAA GAT GGT GAT GGG ATT TC-3′ (SEQ ID NO: 6) (Reverse)” for GAPDH were used in the individual RT-PCR reactions, and the results were normalized against GAPDH mRNA. After denaturation at 95° C. for 30 seconds, annealing at 55° C. for 30 seconds, and elongation at 72° C. for 1 minute (35 cycles) during RT-PCR. The resulting products were then analyzed using agarose gel (2%) electrophoresis.

Giemsa Banding (G-Banding) Karyotyping of Reprogrammed BCMA-Specific iPSC

Karyotyping of reprogrammed BCMA-specific iPSC was evaluated by staining for condensed chromosomes of metaphase cells during active cell division on day 4 after passage. After partial digestion with trypsin, cells were exposed to hypotonic KCL solution and fixed in a methanol/acetic acid solution. The distinct Giemsa banding (G-banding) patterns were evaluated to identify chromosomal abnormalities for each chromosome (Stockert et al. 2014, Moralli et al. 2011) (Cell Line Genetics; Madison, WI).

### BCMA-Specific Embryoid Body Formation

BCMA-specific iPSC (3.0×10⁵) were transferred to individual wells of ultra-low attachment 6-well plates (Corning, Riverfront plaza, NY) and cultured in StemFit Basic02 medium containing 10 ng/ml FGF-basic (Peprotech), 10 μM Rock inhibitor (Y-27632) and 10 μM GSK-3 inhibitor (CHIR99021: R&D systems). To generate EB formation, the culture medium was changed to embryoid body-basal medium (StemPro-34; Gibco-Life Technologies, Rockville, MD) supplemented 2 mM Glutamax (Gibco-Life Technologies), Monothioglycerol (Sigma Aldrich, St. Louis, MO), and a cocktail of 10 μg/ml human insulin, 5.5 μg/ml human transferrin and 5 ng/ml sodium selenite (Invitrogen, Carlsbad, CA). On the following day, a cocktail of cytokines and growth factors including 50 ng/ml BMP4 (R&D systems), 50 ng/ml VEGF (R&D systems), 50 ng/ml bFGF (Peprotech), 50 μg/ml Ascorbic acid 2-phosphate (Sigma Aldrich) was added, the cells were cultured overnight, and ALK5 inhibitor (SB431542: Cayman Chemical, Ann Arbor, MI) was added on the second day of differentiation. The culture media was changed to embryoid body-basal medium containing 50 ng/ml SCF (R&D systems), 50 ng/ml VEGF, 50 ng/ml bFGF, and 50 μg/ml Ascorbic acid 2-phosphate on day 4, and then with additional 30 ng/ml TPO (Peprotech) and 10 ng/ml Flt3L (Peprotech) on day 7 and day 9. On day 11, the presence of CD34⁺HPC were confirmed from the developed embryoid body by flow cytometry.

### Scorecard Assays

Trilineage differentiation and pluripotency potential were assessed for the BCMA-specific iPSC and embryoid body in a comprehensive real-time PCR gene expression assay using TaqMan® hPSC Scorecard™ Panel, which comprised controls, housekeeping, self-renewal, and lineage-specific genes (ThermoFisher, Waltham, MA). In brief, mRNA was purified from the cells using the RNeasy micro kit (QIAGEN), and the complemental DNA was synthesized using a High-Capacity cDNA Reverse Transcription kit (Applied Biosystems, Beverly, MA). The cDNA was combined in qPCR master mix kit, and a quantitative PCR assay was performed using QuantStudio 6 Flex Real-Time PCR Systems (Applied Biosystems). The resulting expression data set was analyzed using the hPSC Scorecard™ Analysis software (ThermoFisher, Waltham, MA), which is compatible with a wide range of Applied Biosystem Real-Time® PCR systems, that compares the expression pattern against a reference standard.

Purification of CD34⁺ Hematopoietic Progenitor Cells (HPC) in Embryoid Body Originated from BCMA-Specific iPSC and Differentiation into T Cells (BCMA-Specific iPSC-T Cells)

On day 11 of embryoid body formation, cells were harvested and stained with a cocktail of fluorochrome-conjugated human mAb specific to CD34, CD14, CD43 and CD235a, and the specific live gated HPC (CD34⁺CD43⁺/CD14⁻ CD235a⁻) were sorted using a FACS Aria II flow cytometer. The sorted HPC were cultured for T-lineage cell differentiation on immobilized Fc-DLL4 chimera protein (10 μg/ml) (Sino Biological, Beijing, China) with Retronectin (10 g/ml) (TaKaRa Bio, Kusatsu-shi, Shiga, Japan), followed by culture in α-MEM medium (Invitrogen) supplemented with 15% FBS, 2 mM Glutamax, 10 μg/ml insulin, 5.5 μg/ml transferrin, 5 μg/ml sodium selenite, 50 μg/ml Ascorbic acid 2-phosphate, 55 μM 2-mercaptoethanol (Invitrogen), 50 ng/ml SCF, 100 ng/ml TPO, 50 ng/ml IL-7, 50 ng/ml Flt3L, 15 nM SDF-1a (Peprotech) and 7.5 nM SB203580 (Sigma Aldrich). The cells in differentiation were harvested and replaced with fresh media containing the fresh cytokines and growth factors listed above, in every two days. In addition, they were transferred onto new Fc-DLL4 and Retronectin-coated wells in a 48-well plate, once a week, for a total of 3 weeks. Finally, the re-differentiated T cells were harvested, stained with a fluorochrome conjugated mAbs specific to CD3, CD4, CD5, CD7, CD8α, CD8β, CD45 and TCRαβ (Beckton Dickinson), fixed in 2% Paraformaldehyde, acquired using a LSRFortessa flow cytometer and analyzed using FACS DIVA v8.0 (Beckton Dickinson) or FlowJo v10.0.7 (Tree star) software.

Phenotypic Analysis of BCMA-Specific iPSC-T Cells and Tumor Target Cells

Phenotypic characterization of BCMA-specific iPSC-T cells was performed by staining them with Live/Dead Aqua and fluorochrome-conjugated anti-human mAbs including CD3, CD4, CD8, CD45RO, CCR7, CD28, CD38, CD69, 41BB, CTLA4, PD1, LAG3, and TIM3. Tumor cell lines (MM cells, breast cancer cells) were stained with fluorochrome-conjugated human mAb specific to BCMA or HLA-A2 to be used, either as target or stimulator cells. After staining, the cells were washed, fixed in 2% paraformaldehyde, acquired using a LSRFortessa flow cytometer, and analyzed using FACS DIVA v8.0 or FlowJo v10.0.7 software.

Specific Cell Proliferation and Functional Activities of BCMA-Specific iPSC-T Cells

BCMA-specific iPSC-T cells were evaluated for their specific functional activities against antigen-matched or antigen-non matched and HLA-A2-positive or HLA-A2-negative target cells. To measure T cells proliferation, cells were labeled with carboxy fluorescein succinimidyl ester (CFSE; Molecular Probes, Eugene, OR), washed extensively, and co-incubated with irradiated (20 Gy) tumor cells or antigen-presenting cells (T2, K562-HLA-A2) pulsed with or without peptide in the presence of IL-2 (10 units/ml). As a control, CFSE-labeled cells were cultured in media alone with IL-2. On days 4, 5, and 6, cells were harvested, stained with Live/Dead Aqua and fluorochrome-conjugated human mAbs specific to CD3 or CD8. The proliferation level of live cells gated CD3⁺/CD8⁺CTL was determined as a reduction in CFSE fluorescence intensity as measured by flow cytometry. For functional activities, the BCMA-specific iPSC-T cells were co-incubated with HLA-A2⁺ or HLA-A2⁻ tumor cells (myeloma patients' bone marrow mononuclear cells, tumor cell lines) in the presence of fluorochrome conjugated CD107a mAb. After 1-hour incubation, a mixture of CD28/CD49d mAb, Brefeldin A and Monensin (BD) were added and incubated for an additional 5 hours. Cells were then harvested, washed, and stained with fluorochrome-conjugated human mAb specific to CD3, CD4, CD8, CD45RO or CCR7. After the surface staining, cells were fixed/permeabilized, stained with intracellularly with Granzyme B, IFN-γ, IL-2 and TNF-α fluorochrome-conjugated human mAbs, washed with Perm/Wash solution (BD), fixed in 2% paraformaldehyde, acquired using a LSRFortessa flow cytometer, and analyzed using FACS DIVA v8.0 or FlowJo v10.0.7 software.

Single-Cell Sequencing of T Cell Receptor (TCR) on BCMA-Specific iPSC-T Cells

TCR sequence analyses were performed on a single cell isolated from BCMA-specific iPSC-T cells using the rhTCRseq protocol (Li et al., 2019). In brief, targeted amplification of TCR transcripts was performed in a 96-well plate format using single cell-amplified cDNA libraries obtained using the NEBNext Single Cell/Low Input cDNA Synthesis & Amplification Module (New England BioLabs E6421L). The specific library was sequenced using MiSeq 300 cycle Reagent Kit v2 on the Illumina sequencing system according to the manufacturer's protocol with 248-neucleotide (nt) read 1, 48-nt read 2, 8-nt index 1, and 8-nt index 2. The sequencing data analysis was performed, based on the method published previously (Li et al., 2019).

### Whole Transcriptome Profiling

For the library preparation, the SMART-Seq V4 Ultra-low input RNA-Seq kit (Takara Bio, Mountain View, CA) was used to synthesize cDNA from the RNA. The cDNA was created from the RNA by priming the 3′ end with the CDS primer, synthesizing the first strand DNA by RT and template switching using the SMARTseq Oligos to create the second strand by Reverse Transcription. The cDNA was amplified by PCR using 11 cycles. To make Illumina libraries, we sheared the cDNA to 150 bp using the Covaris M220 sonicator and then used the SMARTer Thruplex DNA-Seq kit (Takara Bio) to ligate the Illumina adapters and barcodes to the cDNA fragments. The libraries were then sequenced with paired-end 50 base pairs reads targeting 30M read pairs per sample on an Illumina NovaSeq 6000 platform. Sequenced reads were aligned to the UCSC hg19 reference genome assembly and gene counts were quantified using STAR (v2.5.1b) (Dobin et al. 2013). Differential gene expression testing was performed by DESeq2 (v1.10.1) (Love et al. 2014) and normalized read counts (FPKM) were calculated using cufflinks (v2.2.1) (Trapnell et al. 2010). RNAseq analysis was performed using the VIPER snakemake pipeline (Cornwell et al. 2018). The samples were passed the quality control and had 20-40M reads with a >85% map rate in all samples; a majority of reads map to coding sequence and >10,000 protein coding genes were detected in each sample (FPKM>1). In addition, the number of intersecting up-regulated or down-regulated genes were plotted for each set using the R package Venn Diagram. The Deseq2 results tables for each comparison were filtered for up-regulated genes with a log 2 fold change >2 and the p-value set as <0.05 and by down-regulated genes with a log 2 fold change <−2 and the p-value set as <0.05.

### Gene Ontology (GO) Enrichment Analysis

GO functional enrichment analysis, including biological process categories (BP), associated cellular component (CC) and molecular function (MF), was performed to identify functional enrichment of DEGs. The Database for Annotation, Visualization and Integrated Discovery (DAVID 6.8 Feb. 2021) (Huang et al, 2009 (1), Huang et al, 2009 (2)) was used to identify GO categories with the p-value set as <0.05.

### Statistical Analysis

Summary results are presented as the mean±SE. Groups were compared using unpaired Student's t-test. Differences were considered significant when *p<0.05.

### Results

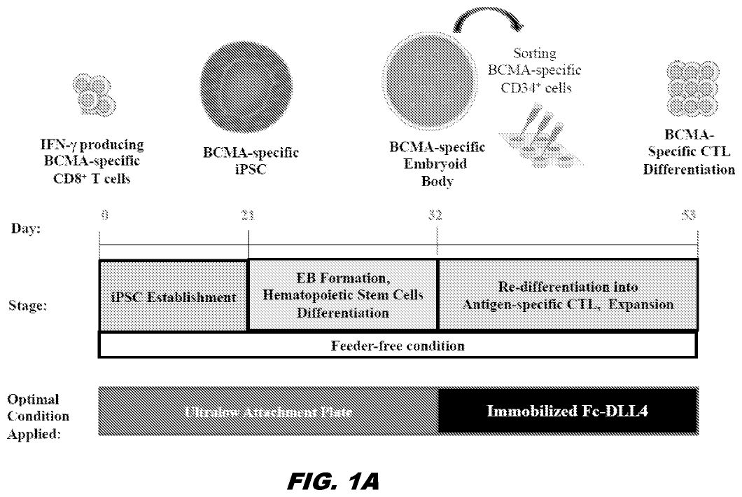

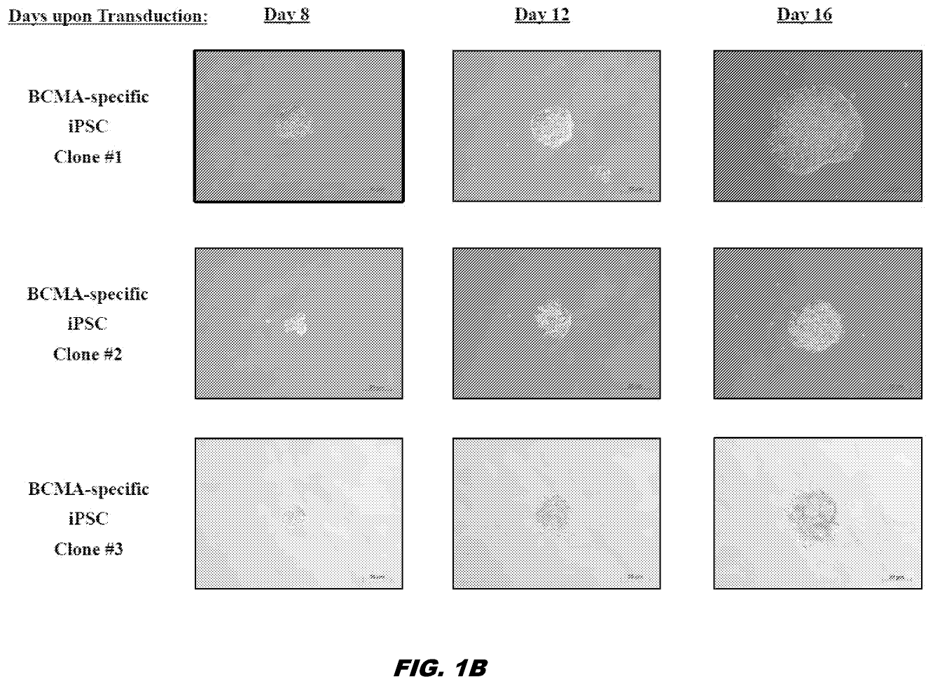

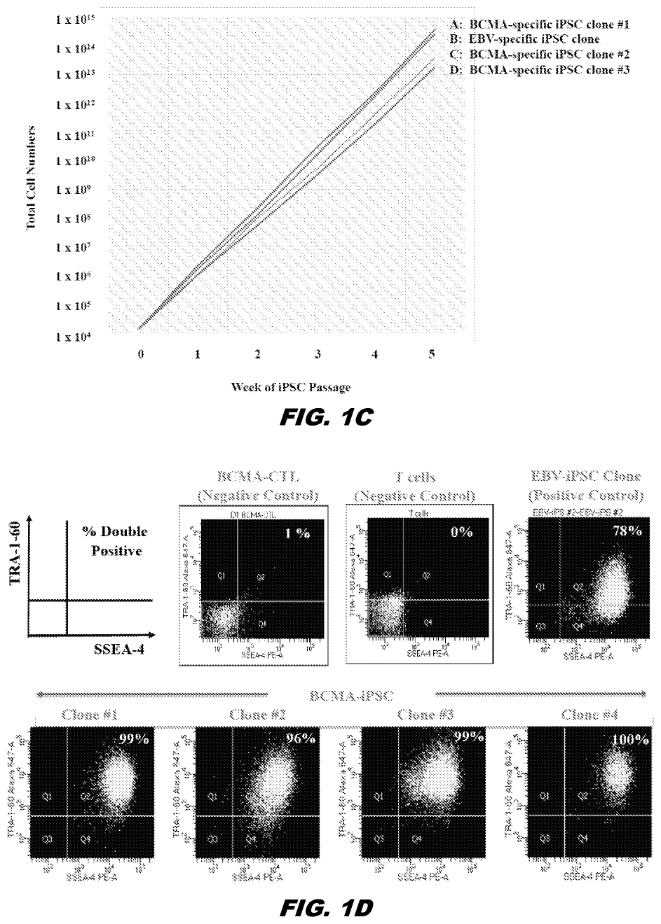

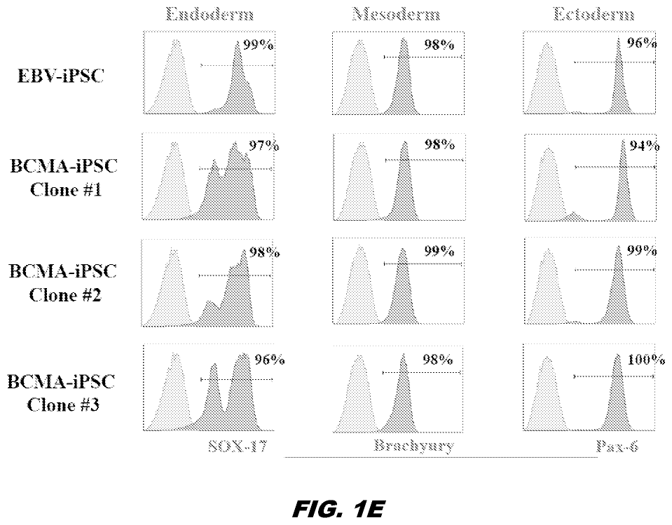

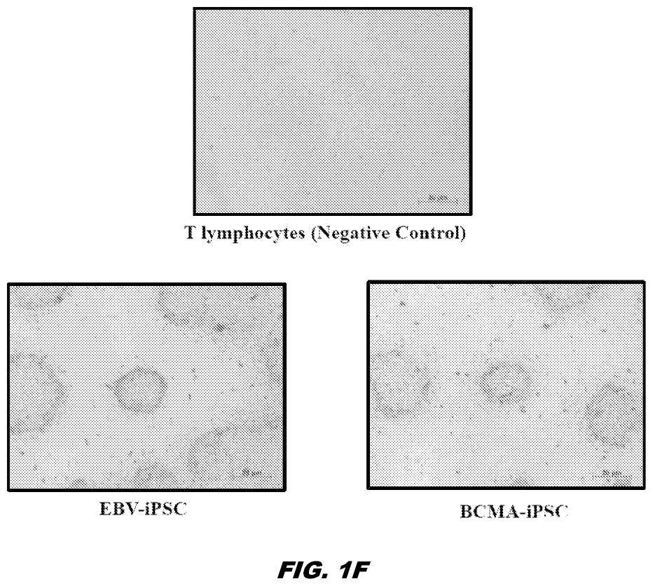

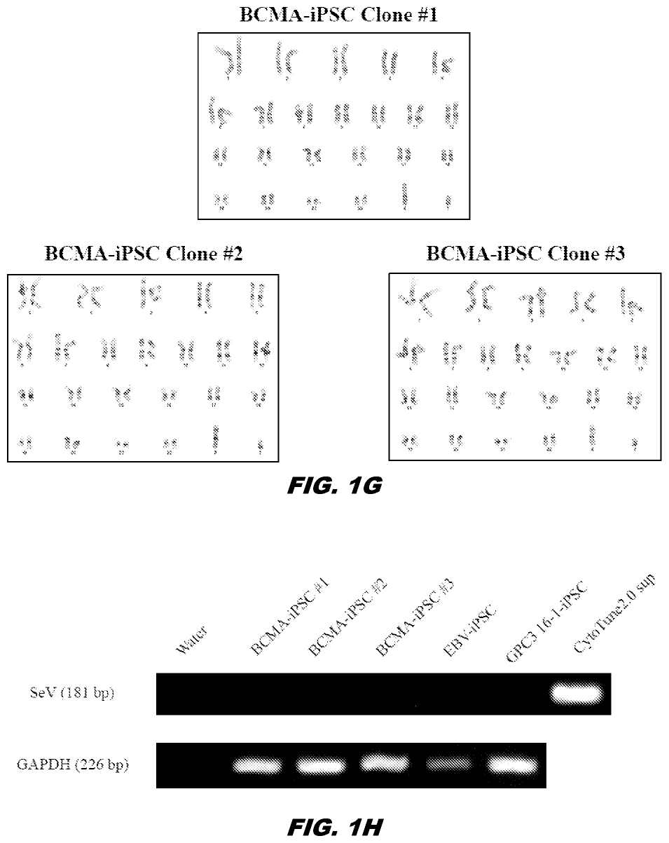

FIGS. 1A-1H. BCMA-Specific iPSC Display Pluripotency Potential and Normal Karyotypes.

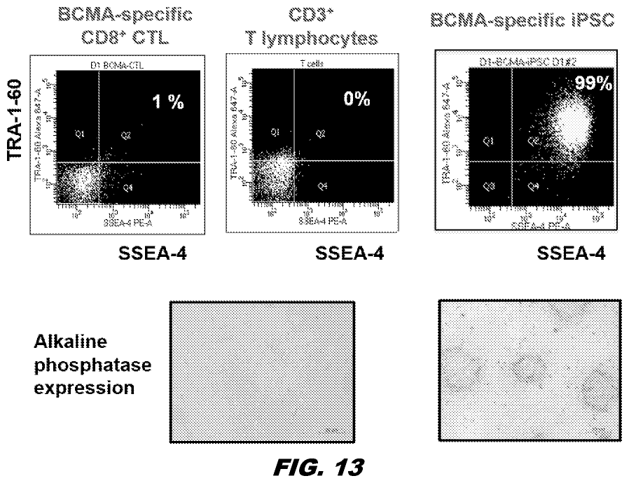

Heteroclitic BCMA₇₂₋₈₀ peptide (YLMFLLRKI (SEQ ID NO: 1))-specific CTL (BCMA-CTL) having characteristic increased expression of T cell activation (CD69⁺, CD38⁺) and co-stimulatory (CD40L⁺, OX30⁺, GITR⁺, 41BB⁺) molecules were generated from HLA-A2⁺ donors' T cells ex vivo as described previously (Bae et al. 2019). Following the BCMA-CTL generation, IFN-γ producing CD8⁺CTL were sorted and used as a source to develop BCMA-specific iPSC. The specific procedures applied in this embodiment, including iPSC induction, embryoid body formation, BCMA peptide-specific CD34⁺ stem cell isolation, and re-differentiation into the antigen-specific CTL, are described in FIG. 1A. In parallel, EBV-specific iPSC which were established using HLA-A2-specific EBV LMP2A426-434 peptide (CLGGLLTMV (SEQ ID NO: 7))-specific CTL (kind gift from Kyoto University, Japan) were used as a positive control to validate the overall antigen-specific iPSC process. Following iPSC induction upon transduction of IFN-+BCMA-CTL with the reprogramming transcription factors (OCT3/4, SOX2, KLF4, c-MYC), we observed the establishment and progressive growth (Day 16>Day 12>Day 8) of BCMA-specific iPSC clones, as shown photomicrographs (100×) taken with an inverted microscope (FIG. 1B). The BCMA-specific iPSC clones continuously proliferated over 5 weeks (10¹⁰ folds increase) under our iMatrix-511 feeder-free culture conditions. The level of cell proliferation for BCMA-specific iPSC clones was equal to or greater than the control EBV-specific iPSC (FIG. 1C), which underscores the potential to make and store stocks of the BCMA-iPSC. Since the generation of iPSC requires three weeks of cell culture, it was important to examine their pluripotency status during their development. We detected that each of BCMA-specific iPSC colonies generated (#1, #2, #3, #4) expressed a high level of stem cell markers, SSEA-4 and TRA-1-60 (96%-100%), compared to the control EBV-specific iPSC clone (78%) (FIG. 1D; see also FIG. 13). In contrast, a control CD8⁺BCMA-CTL (1%) or lymphocytes (0%) which did not go through the reprogramming process did not express the stem cell markers. To establish pluripotency potential, BCMA-specific iPSC colonies were next evaluated for their ability to differentiate into Ectoderm, Mesoderm, and Endoderm. Flow cytometric analyses demonstrated a high expression (94%-100%) of representative germ layer markers, such as SOX-17 on Endoderm, Brachyury on Mesoderm, and Pax-6 on Ectoderm in the BCMA-iPSC clones, which was similar levels to those seen in the control EBV-specific iPSC clone (96%-99%) (FIG. 1E). Expression of these germ layer markers further support the pluripotency potential of the BCMA-specific iPSC. Next, we evaluated the BCMA-iPSC clones for retention of alkaline phosphatase activity during colony formation, which is known to correlate with clonogenic and self-renewal potential of undifferentiated human embryonic stem cells and iPSC (O'Connor et al. 2008, Takahashi et al. 2007, Stefkova et al. 2015).

Immunohistochemistry analyses revealed a high expression of alkaline phosphatase in the BCMA-specific iPSC, as shown as a similar intensity to the EBV-specific iPSC (FIG. 1F). In contrast, control T lymphocytes as somatic cells did not display alkaline phosphatase activity, thereby confirming the specific presence of pluripotency marker in the BCMA-iPSC generated. Finally, BCMA-specific iPSC were evaluated for genomic stability and abnormalities based on changes in Giemsa banding (G-banding) patterns including chromosomal duplications, insertions, deletions, translocations, or centromere loss. Common strategies for iPSC karyotype analyses were used to determine the distinct banding patterns within the chromosomes of metaphase cells (Amit et al. 2010). Cytogenetic analyses of chromosomes by G-banding revealed that the BCMA-specific iPSC clones displayed normal karyotypes (FIG. 1G). After the confirmation of genomic stability, the BCMA-iPSC were evaluated for Sendai virus residue by RT-PCR to determine its loss in the iPSC after multiple passages, following the reprograming process. No residual virus was detected in the BCMA-iPSC, EBV-iPSC, or GPC3 16-1-iPSC, while it was detected within the positive control Sendai virus CytoTune2.0 supernatant (FIG. 111), indicating a clearance of the virus vector from the cells after the transduction process. Taken together, these data confirmed BCMA-specific iPSC pluripotency and self-renewal potential based on the expression of stem cell markers including SSEA-4 and TRA-1-60, the capacity to differentiate into three germ layers, and alkaline phosphatase activity. The BCMA-specific iPSC also displayed a normal karyotype and lacked the residual Sendai viral vector, warranting us to proceed to next steps for the development.

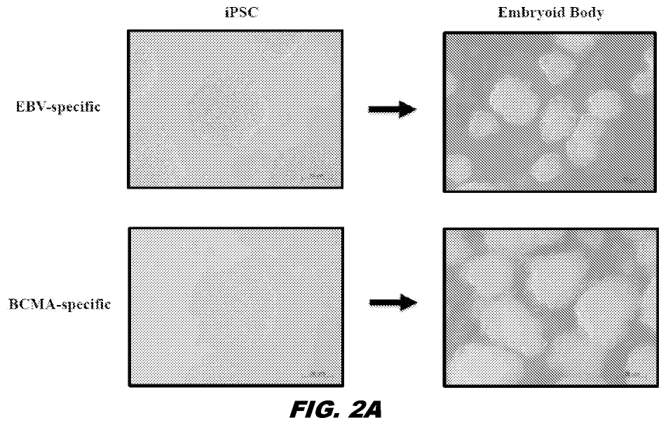

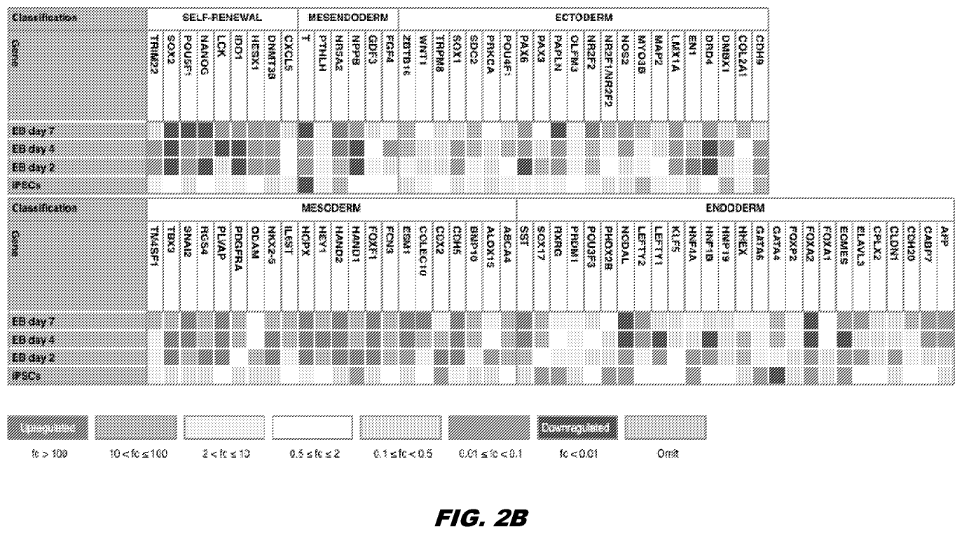

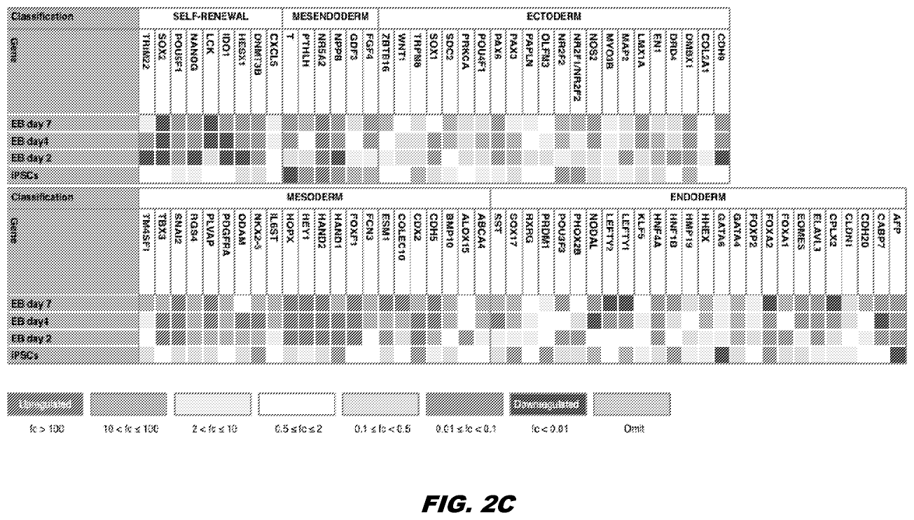

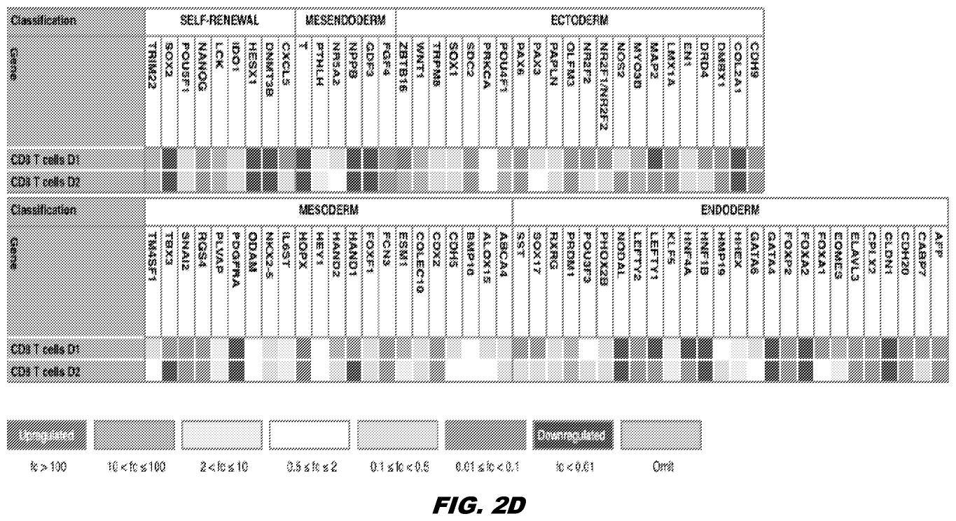

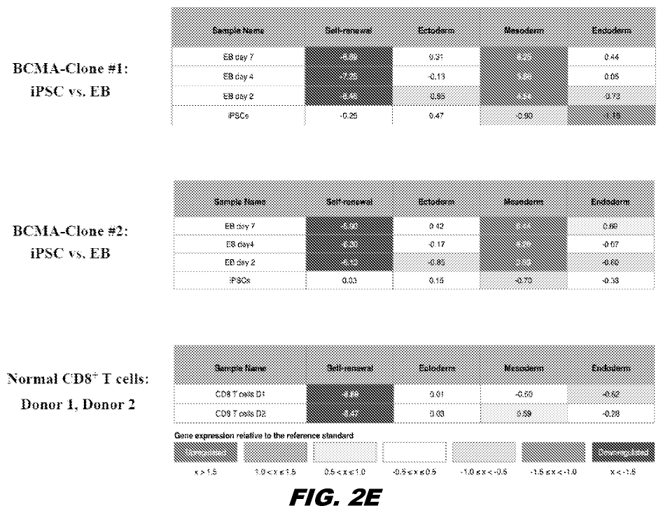

FIGS. 2A-2E. BCMA-Specific iPSC have Polarized into Mesoderm Differentiation During Embryoid Body Formation.

BCMA-specific iPSC and control EBV-specific iPSC underwent embryoid body formation for antigen-specific CD34⁺HPS. The difference in morphology was detected between iPSC and embryoid body formed (day 11) on photomicrographs (100×) taken by inverted microscope, with the similarities between the BCMA and EBV-specific (FIG. 2A). Next, BCMA-specific iPSC pluripotency and germ layer bias and polarization were evaluated during embryoid body formation on days 2, 4, and 7 using ScoreCard analysis, which determined the fold change in gene expression relative to an undifferentiated reference set. During embryoid body formation, we observed gradual increases overall in the genes associated with mesoderm differentiation including TM4SF1, TBX3, SNAI2, RGS4, PLVAP, PDGFRA, NKX2-5, IL6ST, HOPX, HEYl, HAND2, HAND1, FOXF1, FCN3, ESM1, CDX2, CDH5, BMP10 and ABCA4, as shown BCMA-iPSC clone #1 (FIG. 2B) and BCMA-clone #2 (FIG. 2C), but not in CD8⁺ T cells (N=2; Donor 1, Donor 2) as a control representing somatic cells type (FIG. 2D). Gene expression profile were summarized as statistical comparison between iPSC and embryoid body formation measured on day 2, 4 or 7 to that of the undifferentiated reference set. We observed a significant (*p<0.05) upregulation of genes associated with mesoderm differentiation in BCMA-specific clone #1 [6.25>5.86>4.54>−0.90] and BCMA-specific clone #2 [6.44>6.20>3.53>−0.70], but not in control somatic CD8⁺ T cells obtained from two individual donors (FIG. 2E). In contrast, we observed a significant (*p<0.05) down-regulation of the genes contributing to self-renewal but no changes in the genes involved in ectoderm/endoderm differentiation during the embryoid body formation from BCMA-specific iPSC as well as in the control CD8⁺ T cells. Thus, these results provide evidence that the embryoid body formation protocol applied in these studies is highly effective in directing mesodermal lineage development toward blood and non-epithelial cells establishment and supports our process to direct iPSC to T-cell differentiation.

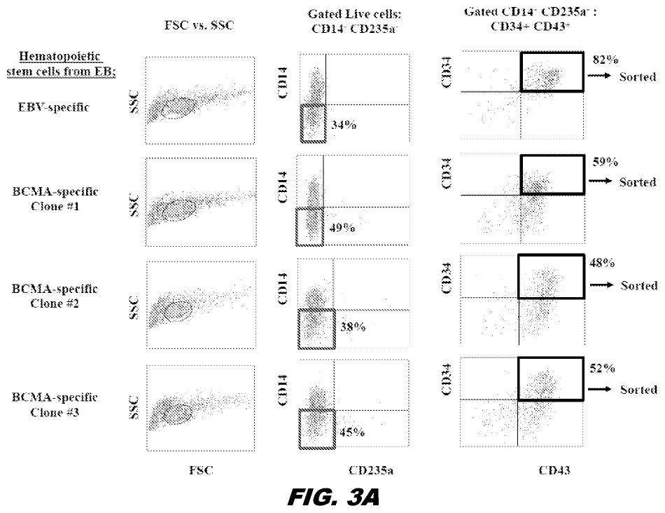

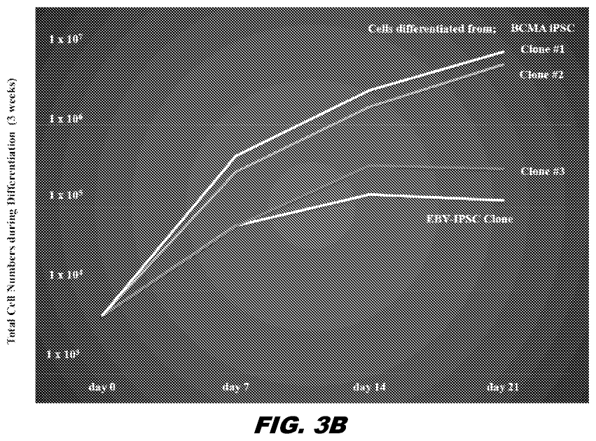

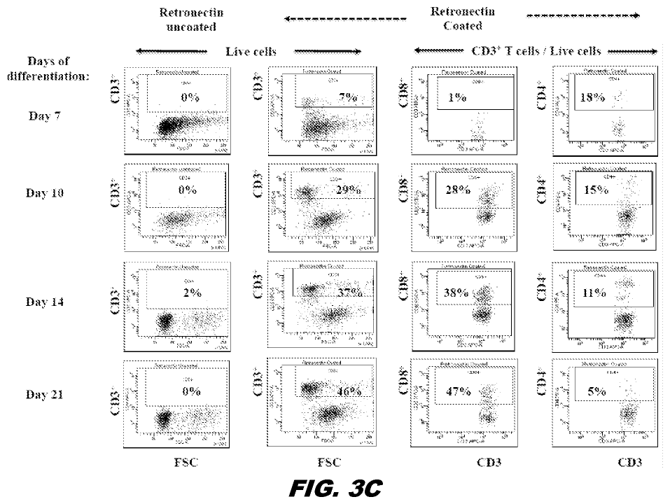

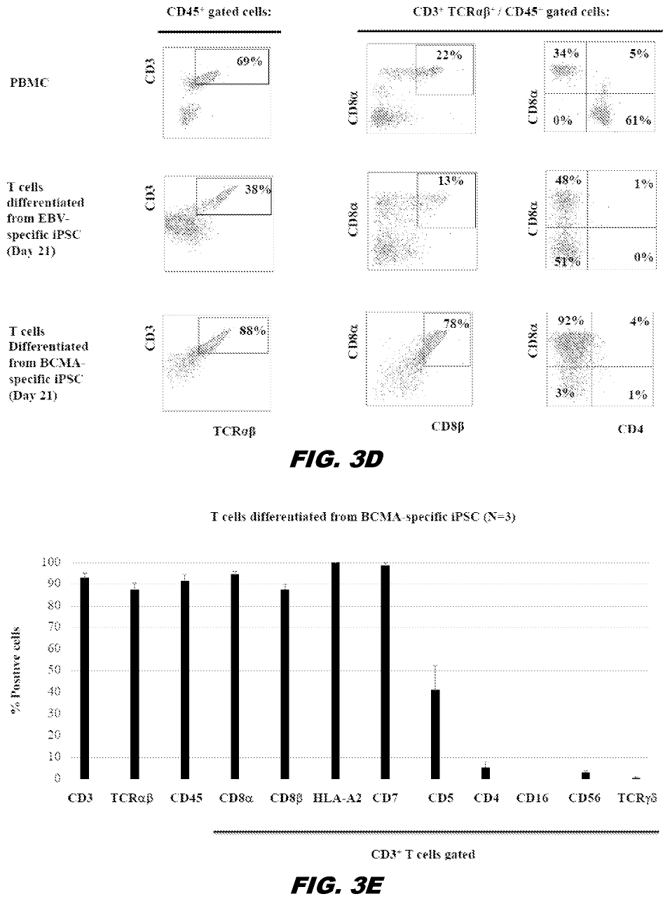

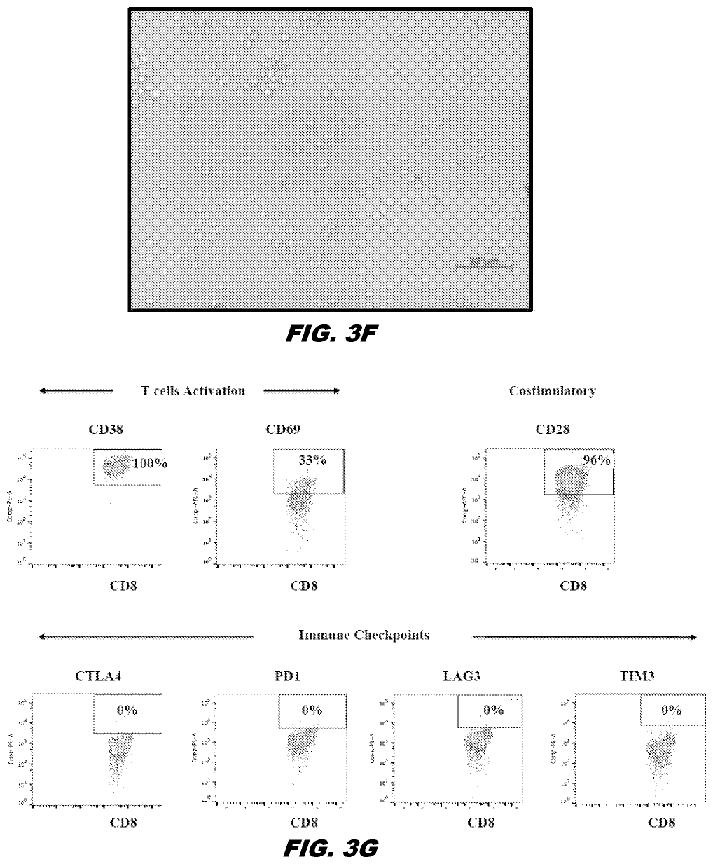

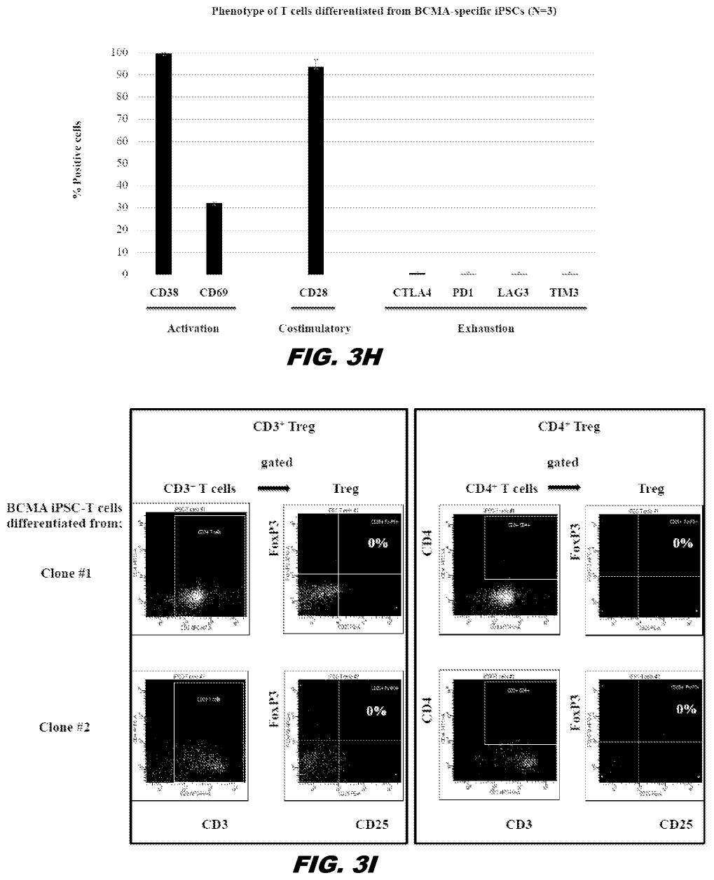

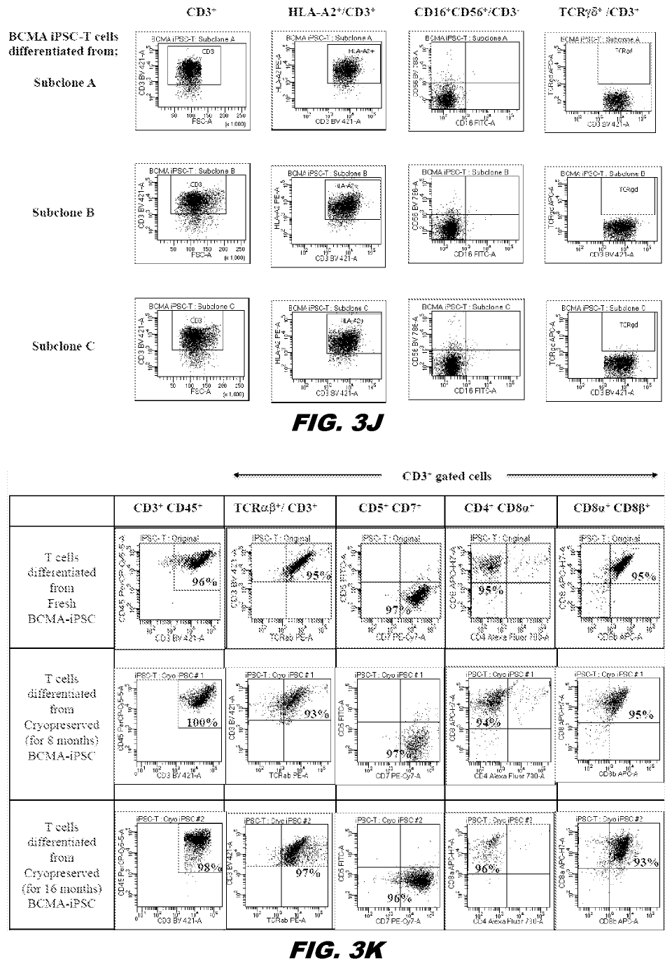

FIGS. 3A-3K the CD34⁺ Hematopoietic Progenitor Cells Isolated from Embryoid Body were Committed into a Specific Cell Subset.

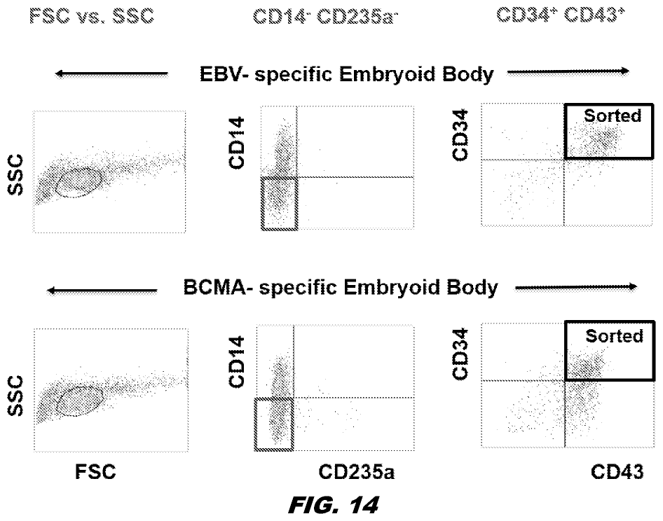

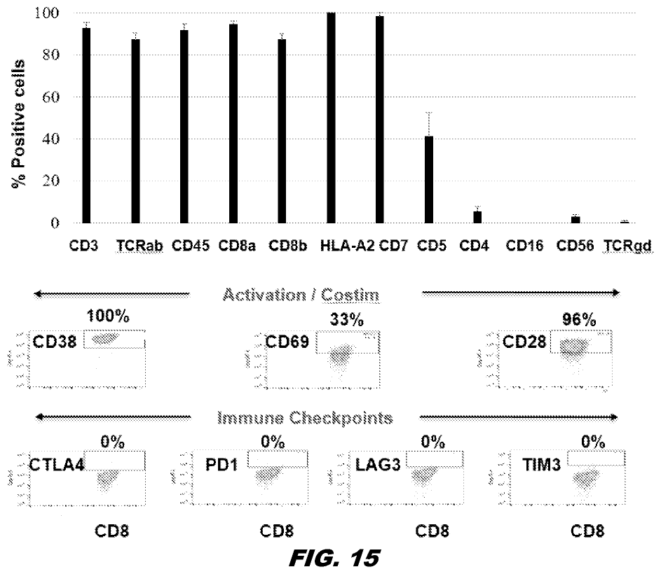

Following the formation of embryoid body, the morphology of distinct cell cluster was detected, and they were further evaluated for phenotypic characterization. The reprogramed hematopoietic progenitor cells (HPC; CD34⁺CD43⁺/CD14⁻ CD235a⁻) in BCMA-specific embryoid body (48-59%) or EBV-specific embryoid body (82%) were sorted using a FACS Aria flow cytometer (FIG. 3A; see also FIG. 14). The HPC (5×10³) sorted from each clone were exposed to Fc-DLL4 signaling in feeder-free culture condition and the changes in cell numbers and phenotype were evaluated for three weeks of time period. Their cell numbers were increased over time up to 1,800 folds on day 21; BCMA-specific clone #1: 5×10⁵, clone #2: 8×10⁶, clone #3: 9×10⁶] or EBV-specific clone (2×10⁵) (FIG. 3B). In addition, their phenotype was gradually changed over time with a differentiation into CD3⁺ T cells [% CD3⁺ T cells: 46% (day 21)>37% (day 14)>29% (day 10)>7% (day 7)] in the presence of retronectin/Fc-DLL4 signal and re-differentiation media. In contrast, no CD3⁺ T cell differentiation was observed when the progenitor cells were not exposed to the retronectin/Fc-DLL4 signaling but cultured in re-differentiation media alone (FIG. 3C). A continuous T cells differentiation was detected into the subset of CD8⁺ Tc cells [47% (day 21)>38% (day 14)>28% (day 10)>1% (day 7)], as aligned with a decrease in subset of CD4⁺ Th cell over time [5% (day 21)>11% (day 14)>15% (day 10)>18% (day 7)]. While the progenitor cells were continuously differentiated into CD3⁺CD8⁺ T cells during the time allowed for differentiation (3 weeks), the frequency of CD4⁺CD8⁺ double positive and immature type of T cells was decreased after an initial increase shown by Day 10. The decrease in the immature T cell population was aligned with a shift to mature CD8⁺ T cells differentiation along with antigen-specific CTL development. Full differentiation of T cells from the progenitor cells was completed by Day 21, which showed a high yield of CD3⁺TCRαβ⁺/CD45⁺ T cells in BCMA-specific iPSC-T cells (88%), which was higher than those in EBV-specific iPSC-T cells (38%) or PBMC (69%) (FIG. 3D). Further phenotypic characterization revealed a high frequency of double positive CD8α⁺CD8β⁺ T cells within the CD3⁺ TCRαβ⁺/CD45⁺ T cells differentiated from BCMA-specific iPSC (78%), which was higher than those differentiated from EBV-specific iPSC (13%) or PBMC (22%). In parallel, a low frequency of double positive CD8α⁺ CD4⁺ cells were detected in CD3⁺ TCRαβ/CD45⁺ T cells within BCMA-specific iPSC-T cells (4%), which was similar to those within EBV-specific iPSC-T cells (1%) or in PBMC (5%) (FIG. 3D). The three BCMA-specific iPSC clones analyzed in this study demonstrated equivalent capacity for embryoid body formation, HPC induction, and re-differentiation into CD3⁺ TCRαβ⁺/CD45⁺ T cells. Full panel of the differentiated BCMA-specific iPSC-T cells from each iPSC clone (N=3) displayed uniform pattern of phenotype including (1) high frequency (˜90%) of T cells and CTL markers (CD3, CD45, CD8α, CD8β, CD7) and T cell receptor (TCRαβ), which are constitutively expressed on normal T cells, (2) lower frequency (˜40%) of CD5⁺ cells, and (3) minimum level (<5%) of T helper cells (CD4⁺), NK cells (CD16⁺, CD56⁺) and TCRγδ T cells. In addition, HLA-A2 molecule expression was maintained highly upon re-differentiation of iPSC to T cells (FIG. 3E; see also FIG. 15). Morphologic evaluations showed that BCMA-specific iPSC-T cells had a very similar cell shape and size compared to normal T lymphocytes, but displayed spindle-like projections which are distinctive (FIG. 3F). These results indicate that the T cells re-differentiation process yielded the desired morphology and phenotypes of T cells including the representing T cells markers, proper TCR rearrangement and MHC molecule expression, which are important for effector T cells for recognition of the tumor (target) cells to respond. The BCMA-specific iPSC-T cells were further examined for their expression of activation and co-stimulatory markers as well as immune checkpoints or induction of regulatory T cells. They were highly activated T cells expressing CD38 (100%), a late T cell activation marker and CD28 costimulatory molecule (94±3%); but they expressed CD69, an early activation marker, in a lower level (˜30%) (FIGS. 3G, 3H), indicating their full activation on antigen-specific T cells. These phenotypic characteristics were consistent with the specific expression detected previously on the parent BCMA peptide-specific CD8⁺CTL (Bae et al. 2019). In contrast, a key difference was detected on the BCMA-specific iPSC-T cells from the parent BCMA peptide-specific CD8⁺CTL, which was a significant reduction in immune checkpoints expression. While BCMA peptide-specific CTL express various immune checkpoints (Bae et al. 2019), BCMA-specific iPSC-T cells showed a complete downregulation of these inhibitory receptors including CTLA4, PD1, LAG3 and TIM3 (FIGS. 3G, 3H), indicating their improved functional activities against tumor. Next, BCMA-specific iPSC-T cells were further evaluated on development of immune suppressor cells during the process of T cells differentiation. The regulatory T cells (CD3⁺CD4⁺CD25⁺FoxP3⁺) was not detected, consistently in the evaluation of BCMA-specific iPSC-T cells differentiated from iPSC clone # or iPSC clone #2 (FIG. 3I). They were further investigated for their T cell differentiation potential upon multiple subcloning. Each of three subclones (A, B, C) demonstrated equivalent capacity to differentiate to CD8⁺ T cells as the original BCMA-iPSC clone, evidenced by high CD3 expression (>95%) with HLA-A2 molecules, but no differentiation (<5%) into NK cells (CD16⁺CD56⁺/CD3⁻) nor expression of TCRγδ on T cells (FIG. 3J). These results confirm that multiple subclones propagated from the original BCMA-specific iPSC maintain the specific commitment pathway to differentiate into CD3⁺ T cells with the defined phenotypic profiles. We further investigated the impact of cryopreservation for the BCMA-iPSC to differentiate to CD8⁺ T cells after a long-term storage at −140° C. The phenotype were evaluated by the level of T cell differentiation from the parent BCMA-iPSC as fresh cells, BCMA-iPSC upon cryopreservation for 8 months and BCMA-iPSC upon cryopreservation for 16 months. Flow cytometric analyses demonstrate the efficiency to differentiate to T cells was similar in BCMA-iPSC between fresh and the long-term cryopreserved samples (FIG. 3K). The final BCMA-iPSC T cell products differentiated from 8-months or 16-month cryopreserved BCMA-specific iPSC clone showed highly enriched (>93%) T cells phenotype, with high frequencies (>95%) of CD3⁺CD45⁺, TCRαβ⁺/CD3⁺, CD7⁺, CD8α⁺ and CD8β⁺ cells and low frequencies (<5%) of CD5⁺ and CD4⁺ cells, which are directly equivalent to the phenotype of T cells differentiated from the parent fresh BCMA-iPSC (FIG. 3K). In summary, these results demonstrate that the T cell re-differentiation process from BCMA-specific iPSC yields high quality of T cell products with optimal upregulation of key molecules including activation/costimulatory, in the absence of immune suppressors such as checkpoints molecules and regulatory T cell population. Furthermore, the process also demonstrated a capacity to maintain T cell differentiation potential into the antigen-specific CD8⁺ memory T cells, following multiple subcloning in long-term cultures under feeder-free conditions or post-thaw after long-term (18 months) cryopreservation at −140° C., which provide additional benefits for clinical application to treat patients in a continuous manner. Taken together, these results support the reprograming iPSC and differentiation processes for therapeutic application of BCMA-specific T cells as a regenerative medicine for treatment of MM patients, when needed in the relapsed patients.

FIGS. 4A-4F. The BCMA-Specific iPSC Commits to CD8⁺CTL Display Genetic Characteristics with Specific Regulation of Transcription Regulators.

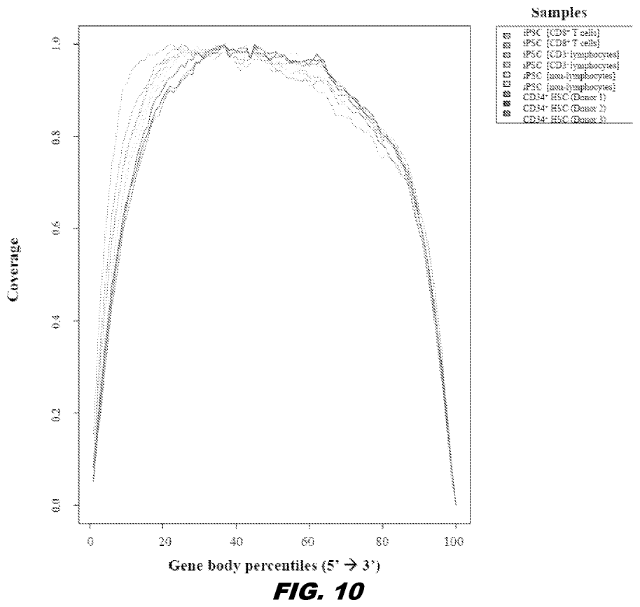

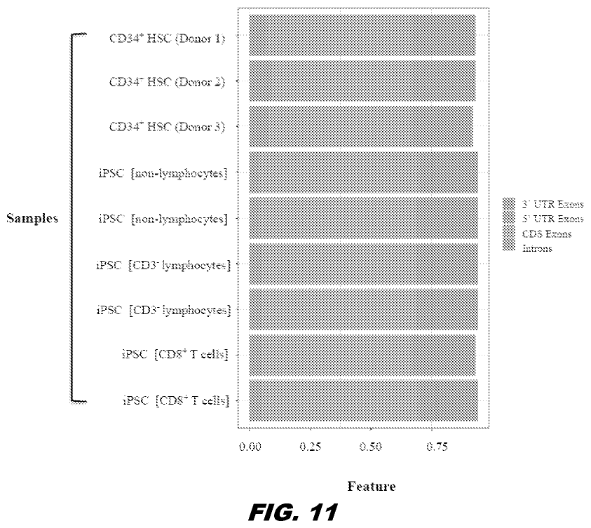

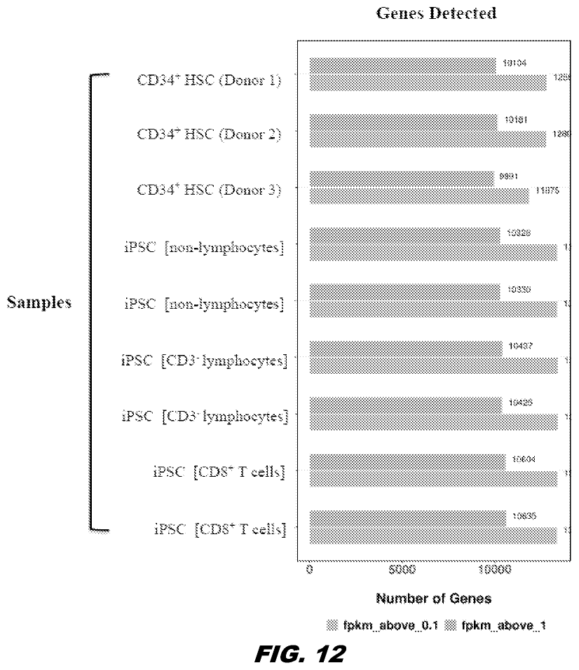

A total of 20 BCMA-specific iPSC clones were established in these studies from BCMA-specific CTL generated from four different HLA-A2⁺ donors. The HPC (CD34⁺CD43⁺/CD14⁻ CD235a⁻) derived from each iPSC clone underwent the procedure to generate T cells with the identical methodology, however their commitment pathway was distinct in following; Group 1—iPSC commit to CD8⁺ T cells (=iPSC [CD8⁺ T cells]), Group 2—iPSC commit to CD3⁻ lymphocytes (i.e., these lymphocytes do not express CD3)(=iPSC [CD3⁻ lymphocytes]) such as B cells, NK or NKT cells, and Group 3—iPSC commit to non-lymphocytes (iPSC [non-lymphocytes]) like monocytes and granulocytes. To characterize each iPSC, especially in their specific transcriptional profiles, the HPC (CD34⁺CD43⁺/CD14⁻ CD235a⁻) from each group of iPSC (N=2 per Group) were sorted and then evaluated by RNAseq analyses, along with CD34⁺HPC from PBMC obtained from three (N=3) normal individuals as control. In these analyses, we first validated and confirmed the quality of RNA purified from each HPC by the viper output analyses (FIGS. 9-12). Upon the confirmation, the RNAseq were pursued for the principal component (PC) analyses to determine the variance within or across the samples with normalized gene expression values (FIG. 4A). The magnitude of PC1 compared with PC2 indicates that there is a much greater transcriptional difference between the iPSC clones and a similarity among the three groups of iPSC with varied differentiation potential. In contrast, the PC2 distinguishes the differences among the iPSC and indicates the iPSC [CD8⁺ T cells] have a strong deviation from other iPSC clones (iPSC [CD3⁻ lymphocytes] and iPSC [non-lymphocytes]). In contrast, scatter plot analyses revealed a very low variance between the replicates within the HPC from each group of iPSC as well as within the HPC from PBMC (N=3), supporting a high similarity in the transcriptional profiles within the replicates (FIG. 4A). The results indicate a low variability of gene transcription profiles within the iPSC clones committed to the identical cell lineage and a higher variability between the groups of iPSC and between iPSC and normal PBMC. Next, hierarchical cluster analyses were performed using the top 1,000 variably expressed genes across the dataset. In evaluation of each HPC samples, four distinct cluster patterns were detected in following in heat maps analyses; Cluster 1—genes upregulated in all BCMA-specific iPSC clones (Sample ID: 1, 2, 3, 4, 5, 6) compared to PBMC (Sample ID: 7, 8, 9), Cluster 2—genes downregulated in BCMA-specific iPSC [CD8⁺ T cells](Sample ID: 1, 2) compared to iPSC [CD3⁻ lymphocytes] and iPSC [non-lymphocytes](Sample ID: 3, 4, 5, 6), Cluster 3—genes upregulated in BCMA-specific iPSC [CD8⁺ T cells] compared to iPSC [CD3⁻ lymphocytes] and iPSC [non-lymphocytes], and Cluster 4—genes downregulated in all BCMA-specific iPSC clones compared to PBMC (FIG. 4B). Genome-wide changes were detected among the different groups of iPSC, however a transition of important genes signaling T cell lineage commitment was distinctly detected in the iPSC [CD8⁺ T cells]. A fewer number of genes were seen in Clusters 2 and 3 [comparisons of HPC among iPSC clones] compared to Clusters 1 and 4 [comparisons of HPC between iPSC clones and PBMC], which indicate the common genetic characteristics and regulation within the iPSC clones than between the iPSC and PBMC. Next, differentially expressed genes were further quantified in the HPC (1) within iPSC clones or (2) between iPSC clones and PBMC. Number of upregulated (Log fold change >2) and downregulated (Log fold change <−2) genes was similar in comparison between iPSC [CD8⁺ T cells] and iPSC [CD3⁻ lymphocytes](upregulated: 485 genes, downregulated: 672 genes) and between iPSC [CD8⁺ T cells] and iPSC [non-lymphocytes](upregulated: 474 genes, downregulated: 480 genes) (FIG. 4C). However, a significantly greater number of genes was detected in comparison of iPSC [CD8⁺ T cells] with CD34⁺HSC (upregulated: 1,154 genes, downregulated: 1,326 genes) [Adjusted P value <0.05], indicating distinctive separated regulation patterns between the iPSC clones and CD34⁺HSC, than within the iPSC clones. Furthermore, the specific genes were identified to further characterize the specific iPSC clones with the cell commitment pathway. A differential expression profile was detected in iPSC [CD8⁺ T cells] compared to iPSC [CD3⁻ lymphocytes], as the upregulation (>1.5 log 2, p adj <0.05) of genes contribute to CD3⁺ T cells development [ITGA1, LCK], CD8⁺ T cells cytotoxicity and activation [GZMB, TNFRSF9, TNFSF10], interferon induction [MX1, IFIT3], mesoderm development [FOXF1, TBX3], common lymphoid progenitor [CD1A], immune response regulation [LTB], and cytokine transporter and protease inhibitor [A2M] as well as the downregulation (<−1.5 log 2, p adj <0.05) of genes that contribute to TGF-β receptor binding [TGFBR3], regulation of cell development and T cell-B cell interactions [CD37] and endothelial cell differentiation [S1PR1](FIG. 4D). In terms of the expression profile in iPSC [CD8⁺ T cells] compared to iPSC [non-lymphocytes], TBX3, FOXF1, GZMB and A2M which contribute to mesoderm development, cytotoxicity and cytokine transport were commonly upregulated (>1.5 log 2, p adj <0.05) on the iPSC [CD8⁺ T cells] compared to iPSC [non-lymphocytes], as shown on its comparison to iPSC [CD3⁻ lymphocytes](FIG. 4E). Furthermore, the differential expression comparison of iPSC [CD8⁺ T cells] with CD34⁺HSC shows the upregulation (>1.5 log 2, p adj <0.05) of genes involved in lymphoid progenitor generation, T cell development, and cytokine transport (CD1A, TL7R, A2M) as well as the downregulation (<−1.5 log 2, p adj <0.05) of genes involved in CD4⁺ Th cell differentiation (KLF2) or regulation of cell development and T cell-B cell interactions (CD37) (FIG. 4F), as commonly detected in the comparison of iPSC [CD8⁺ T cells] with other iPSCs (FIG. 4D, 4E). The differential gene expression profile was further detected in the HPC from iPSC [CD8⁺ T cells] compared to those from PBMC, as updated of those involved in development of effector CD8⁺ T cells (CX3CR1), CD3⁺ T cells (CD3D, LEF1), mesoderm (CDH5, PLVAP) or cytotoxic mediator [NCR2] and cell division (CCNB2), DNA binding and replication [ORC6] or mitotic spindle localization [NUSAP1]. In addition, the genes involved in B cell and T cell rearrangement (DNTT), effector T cells inhibition (LAG3) or CD4⁺ Th cells development (KLF2, SELL) were downregulated in the iPSC [CD8⁺ T cells] in comparison with CD34⁺HPC (FIG. 4F). Thus, these results indicate the specific regulation of genes in the iPSC clones committed to CD8⁺ CTL compared to other iPSC clones with different commitment potential or CD34⁺HPC.

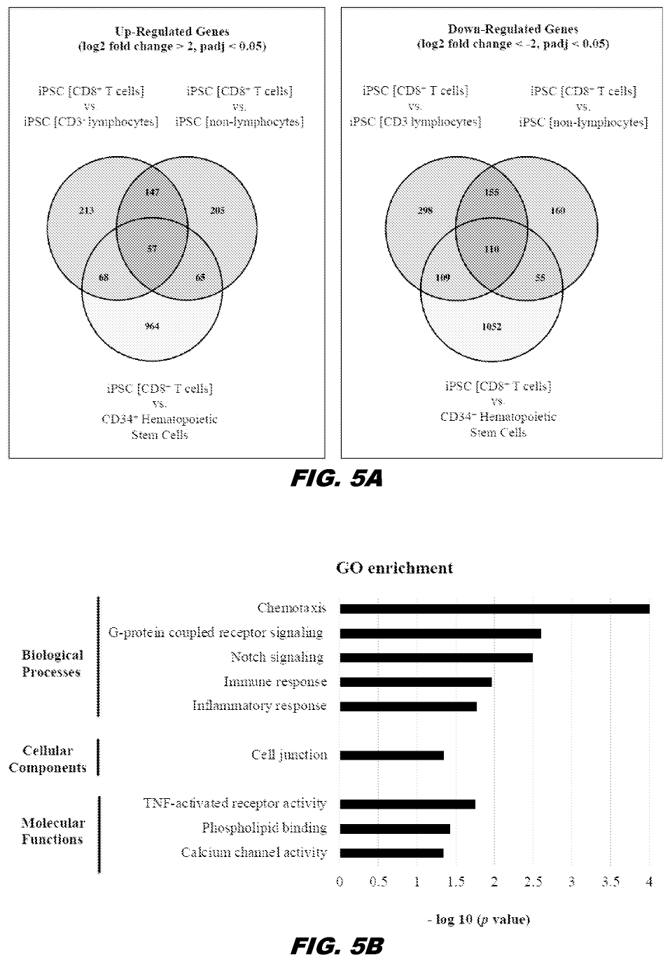

FIGS. 5A, 5B. The iPSC Clones Differentiated into BCMA-Specific CD8⁺ CTL have Commonly Sharing or Distinctly Specific Genes in Comparison with Other iPSC Clones with Different Commitment Pathway.

We characterized specific transcriptional regulation of BCMA-specific iPSC clones with different lineage specific commitment and further evaluated the iPSC clones with CD34⁺ HSC. Specifically, we performed the analyses of three cohorts in following for independent comparison; (1) iPSC [CD8⁺ T cells] vs. iPSC [CD3 lymphocytes], (2) iPSC [CD8⁺ T cells] vs. iPSC [non-lymphocytes], and (3) iPSC [CD8⁺ T cells] vs. CD34⁺HSC. As the results, a total of 1,719 genes were defined for molecular characteristics and they were further evaluated by Venn diagram for the commonly expressed (=sharing) or uniquely expressed (=specific) genes. In the analyses of (1) iPSC [CD8⁺ T cells] vs. iPSC [CD3 lymphocytes] and (2) iPSC [CD8⁺ T cells] vs. iPSC [non-lymphocytes], we detected 204 genes [sum of 147 and 57] as shared upregulated (log 2 fold change >2, padj <0.05) and 265 genes [sum of 155 and 110] as shared downregulated (log 2 fold change <−2, padj <0.05) (FIG. 5A). Importantly, substantial fractions of genes exhibited unique (=specific) expression upon the comparisons among the BCMA-specific iPSC clones, which was higher than common (=shared) expression, in both upregulated and downregulated genes. In further analyses of the iPSC clones with CD34⁺HSC, we detected over 50% of total genes as uniquely (=specific) expressed in the iPSC [CD8⁺ T cells] compared to CD34⁺HSC (964 genes upregulated, 1,052 genes downregulated), whereas only 8˜15% genes belonged to the unique (=specific) expression in the iPSC [CD8⁺ T cells] compared to iPSC [CD3 lymphocytes](213 upregulated genes, 205 downregulated genes) or compared to iPSC [non-lymphocytes](205 upregulated genes, 160 downregulated genes), indicating distinct genetic profiles between iPSC and CD34⁺HSC than among the iPSC clones. We further investigated the genes universally expressed among the iPSC clones to understand the important function for those different types of stems. By overlapping the three cohorts, a total of 167 intersecting genes, including 57 upregulated and 110 downregulated genes, have been identified (FIG. 5A). Next, each of those genes were evaluated for their specific enrichment functional terms via GO annotation analysis of DEGs using DAVID Bioinformatics Resources (Huang et al. 2009 (1), (2)). As the results, several important GO terms were identified, especially in the areas of biological processes, cellular components, and molecular functions as related to chemotaxis, G-protein coupled receptor signaling pathway, Notch signaling pathway, immune response, inflammatory response, cell junction, tumor necrosis factor (TNF)-activated receptor activity, phospholipid binding, and calcium channel activity, which were determined with statistical significance (*p<0.05) and gene counts (≥3) as the cut-off criteria (FIG. 5B). We further identified several important functional genes in the iPSC [CD8⁺ T cells] as upregulated (*p<0.05) having major roles in cell fate decisions, early T cell development; CD8⁺ T cell regulation, differentiation and activation, CD8⁺ memory T cell formation; and immunogenic signal transduction (Table 1).

TABLE 1

| | | | |
|---|---|---|---|
| Upregulated | | | |
| Genes | Aliases | Function, Expression | References |

Cell Fate Decision, Early T cell Development

| | | | |
|---|---|---|---|
| TBX3 | T-box transcription | Cell fate decision | Khan et al. 2020 |
| | factor 3 | toward mesendoderm | Weidgang et al. |
| | | | 2013 |
| HOXA11 | Homeobox All | Early stages of | Stelcer et al. 2019 |
| | | T cell development | Pineault et al. 2019 |

CD8⁺ T Cell Regulation, Differentiation and Activation, CD8⁺ Memory T Cell Formation

| | | | |
|---|---|---|---|
| IRF4 | Interferon Regulatory | Differentiate cytotoxic | Ainsua-Enrich et al. |
| | Factor 4 | effector CD8⁺ T cells, | 2019 |
| | | Form CD8⁺ memory T cells | Huber et al. 2014 |
| PIK3C2B | Phosphatidylinositol-4- | Differentiate Effector/ | Singh et al. 2018 |
| | Phosphate 3-Kinase | Memory T cells, Regulate | Rodríguez-Borlado |
| | Catalytic Subunit | CD4/CD8 T cell differentiation | et al. 2003 |
| | | ratio | |
| KLF15 | Kruppel Like Factor 15 | Develop innate CD8⁺ T cells | Jo et al. 2020 |
| IL-12B | Interleukin 12B | Inhibit B cells differentiation, | Kim et al. 2008 |
| | | Augment CD8⁺ T cell Activation, | Johnston et al. 2013 |
| | | Differentiate Th1 cells | Henry et al. 2008 |
| MAPK4 | Mitogen-Activated | Activate TCR-induced T cells | Marquis et al. 2014 |
| | Protein Kinase 4 | | |

Immunogenic Signal Transduction

| | | | |
|---|---|---|---|
| ITLN ½ | Intelectin ½ | Innate immune defense | Chen et al., 2020 |
| | | | Yi et al. 2017 |
| TRIM6 | Tripartite motif- | Activate type I interferon | Van Tol et al. 2020 |
| | containing protein 6 | signaling pathway | Rajsbaum et al. 2014 |
| EDA2R | Ectodysplasin A2 | Activate NF-kappa-B and JNK | Verhelst et al. 2015 |
| | Receptor | pathways | Sinha et al. 2002 |

A greater number of genes were identified as being downregulated (*p<0.05) than upregulated, with following functions in the iPSC [CD8⁺ T cells]; Cell differentiation and cell cycle regulation; lineage-specific immune cell differentiation; regulation of early embryonic and stem cells development; regulation, organization and development of central nervous system; formation and regulation of vascular system, cytoskeletal rearrangement and angiogenesis; promotion of inflammation, mediation of cellular stress response and homeostasis (Table 2).

TABLE 2

| | | | |
|---|---|---|---|
| Downregulated | | | |
| Genes | Aliases | Function, Expression | References |

Cell Differentiation and Cell Cycle Regulation

| | | | |
|---|---|---|---|
| RPS6KA2 | Ribosomal Protein | Mediate mitogenic activation | Tai et al. 2020 |
| | S6 Kinase A2 | of transcription factors and | |
| | | cellular differentiation | |
| CDK3 | Cyclin Dependent | Regulate G0-G1 and G1-S cell | Ren et al. 2004 |
| | Kinase 3 | cycle transitions | |
| YPEL4 | YIPPEE LIKE 4 | Epithelioid conversion of fibroblasts | Farlie et al. 2001 |

Lineage-Specific Immune Cell Differentiation

| | | | |
|---|---|---|---|
| BATF2 | Basic Leucine | Differentiate lineage-specific | Kayama et al. 2019 |
| | Zipper ATF-Like | cells in immune system | |
| | Transcription Factor 2 | | |
| BTN3A1 | Butyrophilin | Regulate proliferation of activated | Smith et al. 2010 |
| | Subfamily 3 | T cells and release of cytokines | |
| | Member A1 | | |
| USP44 | Ubiquitin Specific | Promote Treg Function, Regulate | Stemeier et al. |
| | Peptidase 44 | spindle assembly checkpoint and | 2007 |
| | | mitotic checkpoint | Yang et al. 2020 |
| CD70 | CD27L, TNSF7 | Regulate activation of B-cells | Al Sayed et al. |
| | | and Natural Killer cells | 2017 |
| | | | Arens et al. 2004 |
| ZXDA | Zinc Finger X- | Cooperate with CIITA to promote | Al-Kandari et al. |
| | Linked Duplicated A | transcription of MHC class I and class II | 2007 |

Regulation of Early Embryonic and Stem Cells Development

| | | | |
|---|---|---|---|
| FGFR1 | Fibrobalst Growth | Regulate embryonic development, | Botrus et al. |
| | Factor Receptor 1 | neural outgrowth, cell | 2021 |
| | | differentiation and migration | |
| NPM2 | Nucleoplasmin-2 | Regulate chromatin reprogramming | Chen et al. |
| | | during fertilization and early | 2019 |
| | | embryonic development | |
| GGN | Gametogenetin | Localize proliferating germ cells | Zhang et al. |
| | | and regulates spermatogenesis | 2005 |
| SPAG1 | Sperm Associated | Regulate fertilization and cytoplasmic | Chagot et al. |
| | Antigen 1 | assembly of the ciliary dynein arms | 2019 |
| CATSPER2 | Cation Channel | Facilitate calcium-dependent responses | Luo et al. |
| | Sperm Associated 2 | essential for fertilization | 2019 |

Regulation, Organization and Development of Central Nervous System

| | | | |
|---|---|---|---|
| N4BP3 | NEDD4 Binding | Develop anterior structures including | Kiem et al. 2017 |
| | Protein 3 | eye, brain and cranial cartilage | |
| P2RY14 | Purinergic | Regulate stem cell compartment and | Kook et al. 2017 |
| | Receptor P2Y14 | neuroimmune function | |
| NLGN2 | Neurologin 2 | Form, maintain, and remodel synapses | Ali et al. 2020 |
| | | of the central nervous system | |
| SHC2 | SHC Adaptor | A signaling adapter that couples | Terui et al. 2005 |
| | Protein 2 | activated growth factor receptors to | |
| | | signaling pathway in neurons | |
| GRASP | General Receptor | Mediate intracellular trafficking and | Pandey et al. |
| | for | macromolecular organization of | 2020 |
| | Phosphoinositides | metabotropic glutamate receptors at | |
| | 1 Associated | synapses | |
| | Scaffold Protein | | |
| AMIGO2 | Adhesion | Enable depolarization-dependent | Ono et al. 2003 |
| | molecule with Ig | neuronal survival; mediates tumoral | Park et al. 2015 |
| | Like Domain 2, | survival, angiogenesis, and metastasis | |
| | Alvin-1, ALI1 | | |
| TBC1D32 | TBC1 Domain | Mediate Shh responses in the | Wang et al. 2018 |
| | Family Member 32 | developing neural tube | |
| CACNA1A | Calcium Voltage- | Regulate neurotransmitter release, | Gandini et al. |
| | Gated Channel | hormone release, or muscle | 2021 |
| | Subunit Alpha 1 A | contraction via calcium channels | |
| SLC6A9 | Solute Carrier | Regulate Glycine in NMDA receptor- | Marques et al. |
| | Family 6 Member 9 | mediated neurotransmission | 2020 |

Formation and Regulation of Vascular System, Cytoskeletal Arrangement and Angiogenesis

| | | | |
|---|---|---|---|
| HEYL | Hes Related Family | Develop cardiovascular system | Kathiriya et al. |
| | BHLH Transcription | | 2004 |
| | Factor with YRPW | | |
| | Motif (SEQ ID NO: | | |
| | 8) Like | | |
| NEURL | Neuralized E3 | Promote endocytosis and neurite | Zhang et al. 2017 |
| | Ubiquitin Protein | growth | |
| | Ligase 1 | | |
| RAB39B | RAB39B Member | Involve in vesicular trafficking | Lesage et al. 2015 |
| | RAS Oncogene | | |
| | Family | | |
| ANK1 | Ankryin 1 | Links integral membrane proteins and | Li et al. 2020 |
| | | underly spectrin-actin cytoskeleton | |
| PSD | Pleckstrin and Sec7 | Induce cytoskeletal remodeling | Macia et al. 2019 |
| | Domain Containing | | |
| LRRK1 | Leucine Rich | Regulate bone mass | Si et al. 2018 |
| | Repeat Kinase, | | |
| | KIAA1790 | | |
| RUNX2 | Runt-Related | Osteoblastic differentiation and | Komori et al. 2017 |
| | Transcription | skeletal morphogenesis, regulatory | |
| | Factor 2 | factors involved in skeletal gene | |
| | | expression | |
| CXCL5 | C-X-C Motif | Recruit neutrophils, promote | Korbecki et al. |
| | Chemokine | angiogenesis and remodel | 2021 |
| | Ligand 5 | connective tissues | |

Promotion of Inflammation, Mediation of Cellular Stress Response and Homeostasis

| | | | |
|---|---|---|---|
| SEMA7A | Semaphorin 7A | Promote inflammation and focal | Suzuki et al. 2007 |
| | | adhesion complex formation, Activate | |
| | | kinase PTK2/FAK1 and | |
| | | MAPK/MAPK3 | |
| JDP2 | Jun | Mediate cellular stress response and | Kimura et al. 2008 |
| | Dimerization | apoptosis, Repress transactivation | Jin et al. 2002 |
| | Protein 2 | mediated by Jun family of proteins | Wuputra et al. 2021 |
| PLA2G6 | Phospholipase | Mediate cellular membrane | Larsson et al. 1998 |
| | A2 Group VI | homeostasis, mitochondrial integrity and | Larsson et al. 1999 |
| | | cell stress | Guo et al. 2018 |
| MAP3K9 | Mitogen-Activated | Mediate mitochondrial death signaling | Xu et al. 2001 |
| | Protein Kinase | pathway, including release cytochrome | |
| | Kinase Kinase 9 | c, leading to apoptosis | |
| PIPOX | Pipecolic Acid | Protect against oxidative stress | Patterson et al. |
| | and Sarcosine | | 2017 Natarajan et |
| | Oxidase | | al. 2017 |
| TNFRSF6B | TNF Receptor | Protect against apoptosis, neutralize | Hsieh et al. 2017 |
| | Superfamily | cytotoxic ligands TNFS14/LIGHT, | Zampino et al. 2020 |
| | Member 6b | TNFSF15, TNFSF6/FASL | |

Therefore, these results demonstrate specific transcriptional regulators contributing to formation and maintenance of the iPSC which specifically commit to CD8⁺ T cells, indicating the application of those genes to orient the development of cytotoxic T lymphocytes.

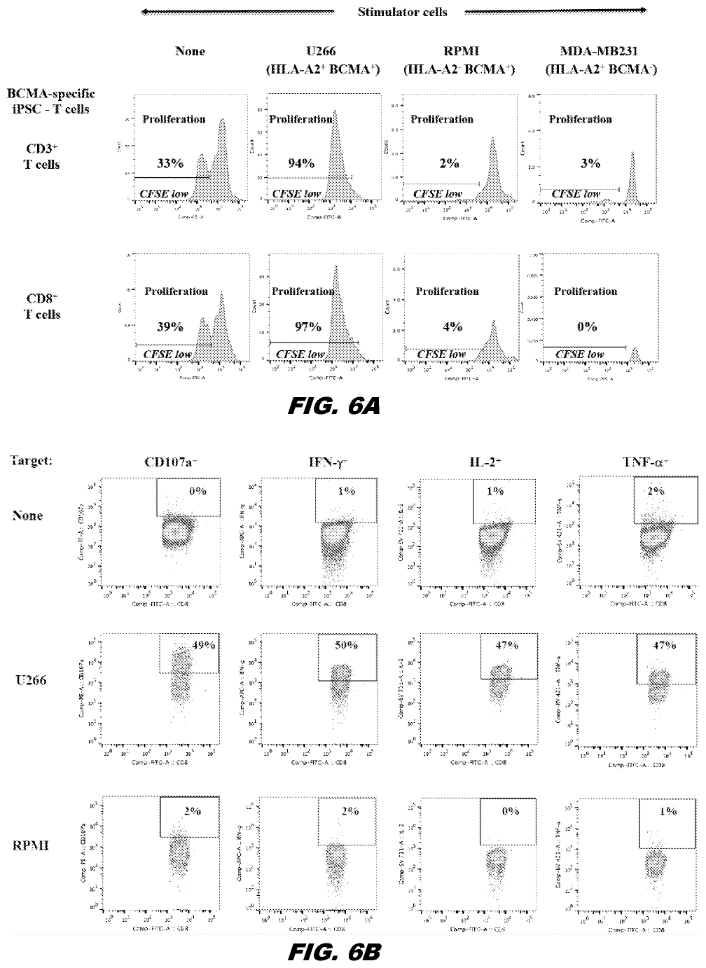

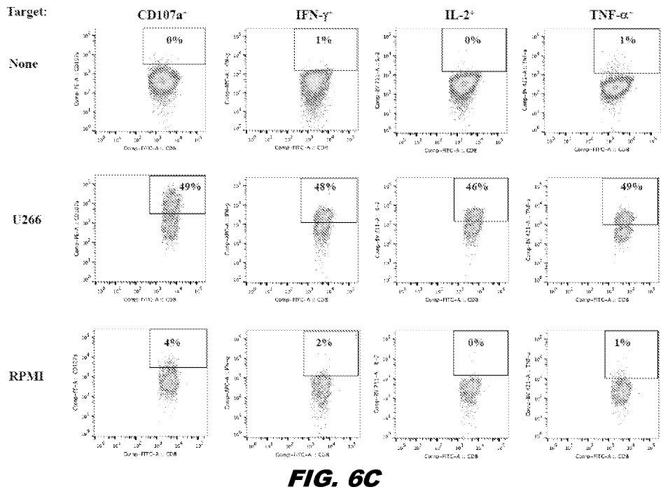

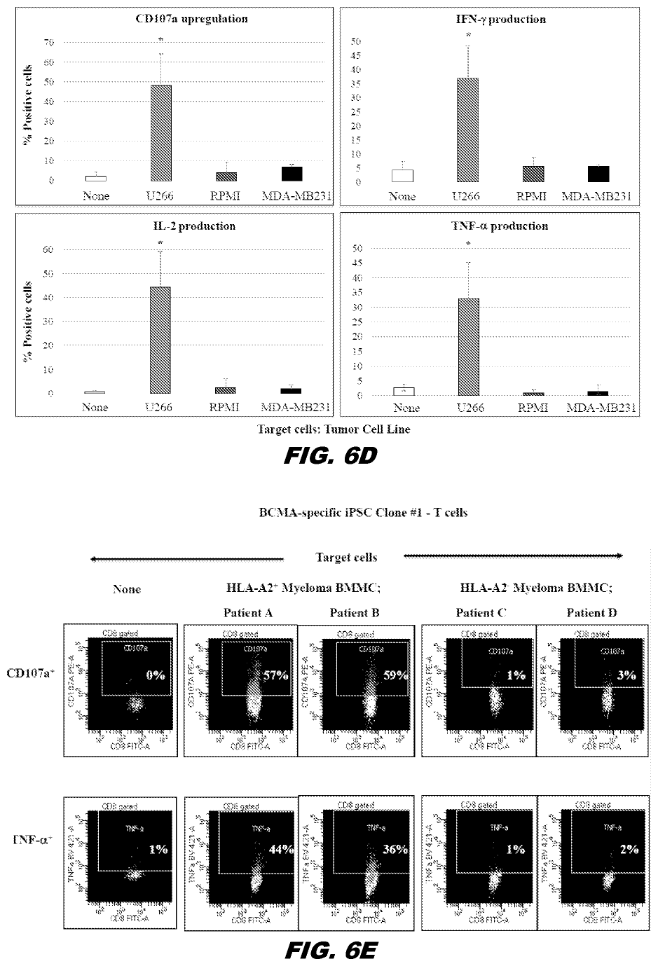

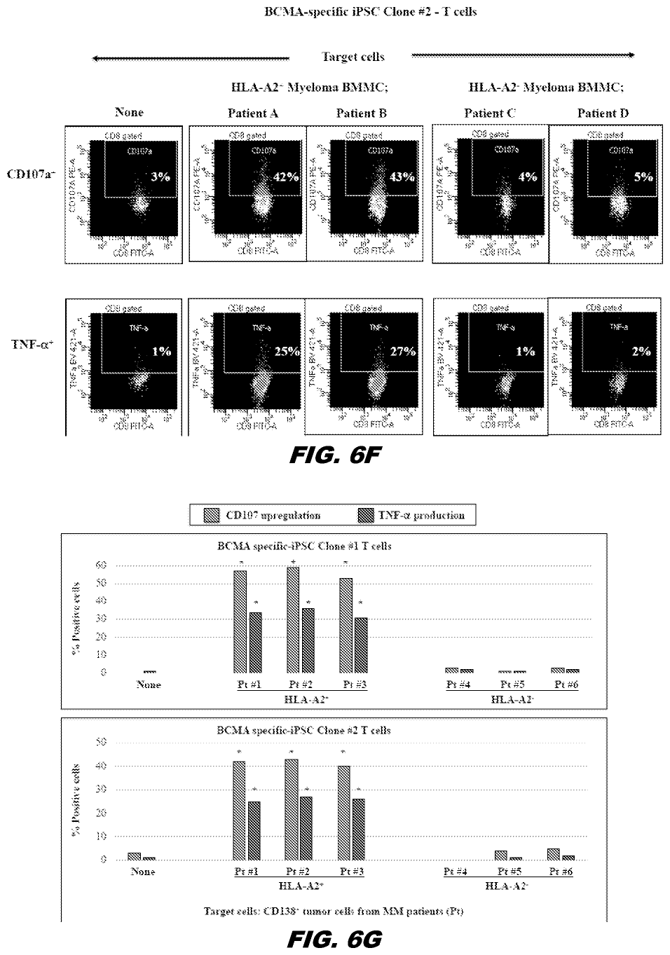

FIG. 6A-6G. Rejuvenated BCMA-Specific iPSC-T Cells are Highly Proliferative to MAI Cells Expressing BCMA and Induce Anti-Tumor Activities in Antigen-Specific and HLA-A2-Restricted Manners.

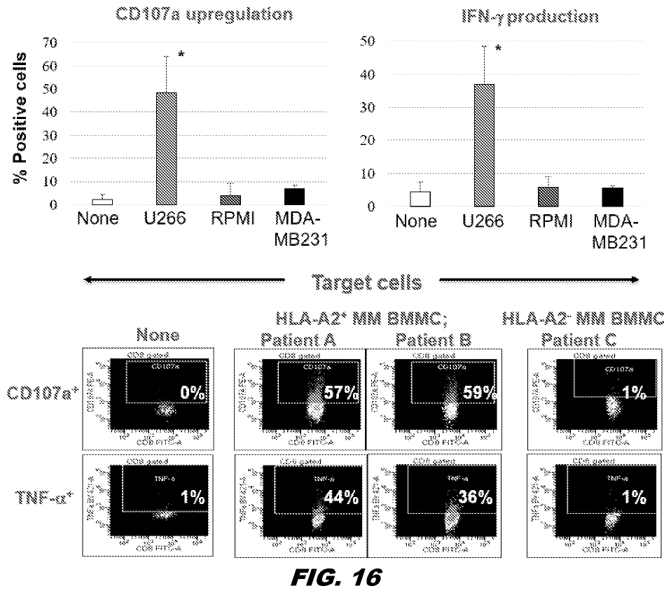

Functional activities of BCMA-specific T cells differentiated from iPSC (iPSC-T cells) were evaluated for their anti-tumor and specific immune responses against MM cells. As target cells, BCMA and HLA-A2 expressing or non-expressing tumor cells (cell lines, primary cells) were tested for the activity of effector T cells in the antigen-specific and the MHC restricted manners. In CFSE-based proliferation assay, a significant increase was detected in expansion of iPSC-T cells, in both CD3⁺ (94% proliferation) and CD8⁺ (97% proliferation) cells, in response to both antigen and MHC matched U266 MM cells [BCMA⁺ HLA-A2⁺] as compared to antigen match and MHC mis-matched RPMI MM cells [BCMA⁺ HLA-A2⁻](CD3⁺: 2% proliferation, CD8⁺: 4% proliferation) nor antigen mis-matched and MHC matched MDA-MB231 breast cancer cells [BCMA⁻HLA-A2⁺](CD3⁺: 3% proliferation, CD8⁺: 0% proliferation) (FIG. 6A). These results indicate the BCMA-specific iPSC-T cells show the specific response to MM cells with the CD3⁺CD8⁺CTL proliferation in antigen-specific and HLA-A2-restricted manners. Next, we further investigated the BCMA specific iPSC-T cells for their specific cytotoxic activities and Th1-type cytokine production against MM cells. The T cells differentiated from BCMA iPSC Clone #1 (FIG. 6B) or BCMA iPSC Clone #2 (FIG. 6C) showed the HLA-A2-restricted anti-tumor activities and specific immune responses in similar levels, as indicated by CD107a degranulation (Clone #1: 49%, Clone #2: 49%) and IFN-γ (Clone #1: 50%, Clone #2: 48%), IL-2 (Clone #1: 47%, Clone #2: 46%) and TNF-α (Clone #1: 47%, Clone #2: 49%) production in response to U266 MM cells [BCMA⁺ HLA-A2⁺], but not to HLA-A2 mis-matched RPMI MM cells. In further analyses of the iPSC-T cells differentiated from BCMA-specific iPSC clones (N=3), we consistently observed their significant (*p>0.05) increases in anti-tumor activities against U266 MM cells [BCMA⁺ HLA-A2⁺] on induction of CD107a degranulation (50%±16%) and production of IFN-γ (37%±12%), IL-2 (44%±15%) and TNF-α (33%±12%) from the baseline level (no stimulation with tumor cells) (FIG. 6D; see also FIG. 16). In contrast, the activities of BCMA specific iPSC-T cells to RPMI MM cells [BCMA⁺HLA-A2⁻] or MDA-MB231 breast cancer cells [BCMA⁻ HLA-A2⁺] remained as the baseline levels (>5%). The functional activities of BCMA specific iPSC-T cells were further investigated against primary CD138⁺ tumor cells isolated from MM patients. The BCMA specific iPSC-T cells displayed robust anti-MM activities against the primary CD138⁺ tumor cells from HLA-A2⁺ MM patients as measured by CD107a degranulation and TNF-α production, upon differentiation from iPSC clone #1 (CD107a⁺: 57% or 59%, TNF-α⁻: 44% or 36%) or iPSC clone #2 (CD107a⁺: 42% or 43%, TNF-α⁺: 25% or 27%) in the evaluation of BMMC from HLA-A2⁺ MM Patient A (FIG. 6E; see also FIG. 16) or Patient B (FIG. 6F), but not in BMMC from HLA-A2⁻ MM Patient C or Patient D (<5%) (FIGS. 6E, 6F). The HLA-A2 restricted anti-tumor activities and immune responses were consistently observed in the BCMA-specific T cells differentiated from iPSC clone #1 or iPSC clone #2, as shown the distinct responses to CD138⁺ tumor cells from HLA-A2⁺ MM patients (N=3), but not to those from HLA-A2⁻ MM patients (N=3) (FIG. 6G). Importantly, the level of anti-tumor activities by BCMA-specific iPSC-T cells were higher than the parent heteroclitic BCMA₇₂₋₈₀ peptide-specific CTL against BMMC from HLA-A2⁺ MM patients (Bae et al. 2019), which could be associated with the rejuvenation of T cells differentiated from iPSC, as evidenced by downregulation of immune checkpoints and absence of regulatory T cells. Thus, these data demonstrate that the selected BCMA-iPSC clones have unique capacity to generate the antigen-specific T cells effectively with high levels of anti-MM activities including CTL proliferation, CD107a degranulation and Th1-type cytokine production, supporting the benefit and therapeutic application to treat MM patients.

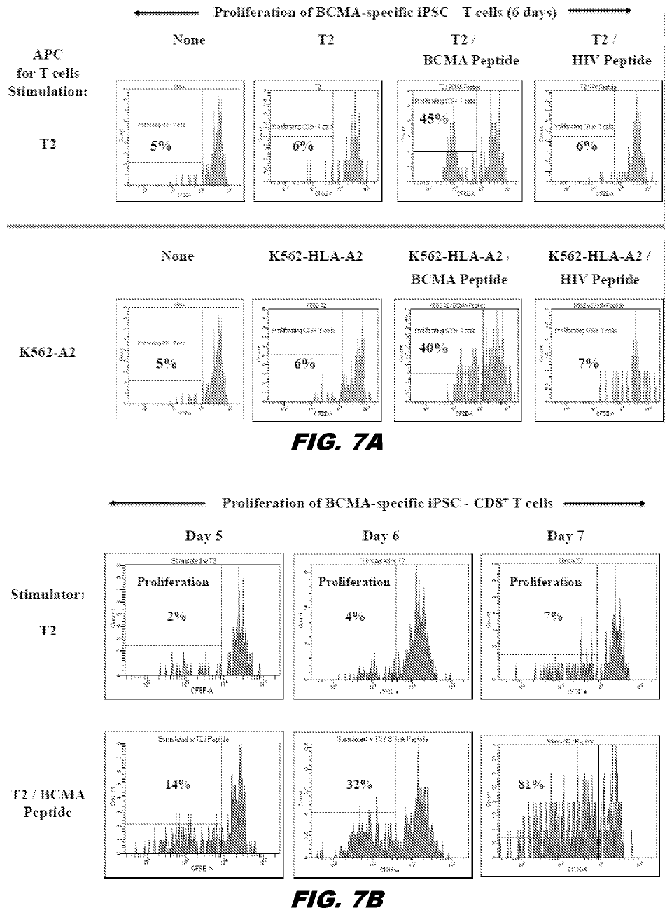

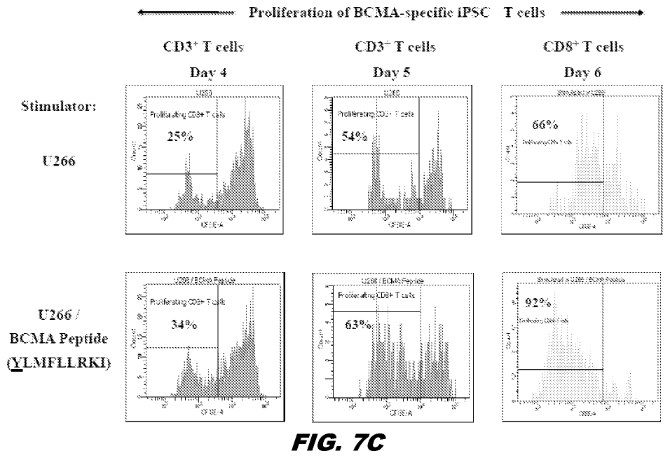

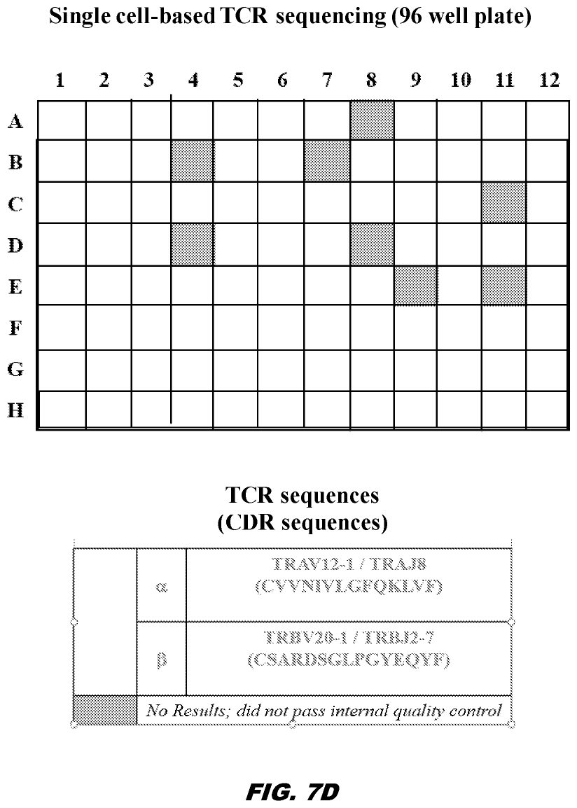

FIG. 7A-7D. BCMA-Specific iPSC-T Cells Demonstrate the Peptide Specific Immune Responses to Heteroclitic BCMA₇₂₋₈₀ (YLMFLLRKI (SEQ ID NO: 1)) and Display a Distinct One TCR Clonotype.

Following the confirmation of functional anti-tumor activities of BCMA-specific iPSC-T cells to HLA-A2⁺ MM cells, they were further investigated for their specific T cell immune responses and CTL proliferation. CFSE-based assays were performed and measured the specific proliferation of BCMA iPSC-T cells in response to relevant (BCMA-derived) or irrelevant (HIV-derived) peptide specific to HLA-A2, upon pulsing of each type of antigen-presenting cells (APC; T2, K562-A*0201), as demonstrated in following four groups including proper controls; (1) iPSC-T cells alone, (2) iPSC-T cells stimulated with no peptide pulsed T2 or K562-A*0201 cells, (3) iPSC-T cells stimulated with HLA-A2-specific and relevant BCMA peptide (heteroclitic BCMA₇₂₋₈₀; YLMFLLRKI (SEQ ID NO: 1)) pulsed T2 or K562-A*0201 cells, and (4) iPSC-T cells stimulated with HLA-A2-specific but irrelevant HIV peptide (HIV-Gag₇₇₋₈₅; SLYNTVATL (SEQ ID NO: 2)) pulsed T2 or K562-A*0201 cells. Representative flow cytometric analyses (FIG. 7A) showed a minimum level of CD3⁺ T cells proliferation (5˜7%) in response to the APC alone or irrelevant HLA-A2-specific HIV-Gag₇₇₋₈₅ peptide (SLYNTVATL (SEQ ID NO: 2)) pulsed APC, while an increased CD3⁺ T cells proliferation was detected in response to the relevant heteroclitic BCMA₇₂₋₈₀ (YLMFLLRKI (SEQ ID NO: 1)) pulsed APC, both in T2 cells (45%) and K562-A*0201 cells (40%), on day 6 of culture. The specific response of iPSC-T cells to the corresponding BCMA peptide was seen in a time-dependent manner, as a gradual increase in CD8⁺ T cells proliferation on day 5 (14%), day 6 (32%) and day 7 (81%) to T2 cells pulsed with the HLA-A2 specific heteroclitic BCMA₇₂₋₈₀ (YLMFLLRKI (SEQ ID NO: 1)) peptide, as compared to baseline response to T2 cells alone without a peptide pulse (2˜7%) (FIG. 7B). The specificity of BCMA iPSC-T cells was further examined in response to U266 MM cells expressing HLA-A2, with or without additional pulse of the HLA-A2-specific heteroclitic BCMA₇₂₋₈₀ (YLMFLLRKI (SEQ ID NO: 1)) peptide. The proliferation level of both CD3⁺ and CD8⁺ T cells was further increased by co-culture of BCMA iPSC-T cells with the BCMA₇₂₋₈₀ peptide pulsed U266 MM cells compared to U266 MM cells alone, as demonstrated a gradual increase in T cell proliferation on day 4 (stimulator; no peptide pulsed U266 vs. BCMA peptide pulsed U266: 25% vs. 34% CD3⁺ T cells), day 5 (52% vs. 73% CD3⁺ T cells) and day 6 (66% vs. 92% CD8⁺ T cells) (FIG. 7C), indicating that BCMA-specific iPSC-T cells display the specific CD8⁺CTL immune responses to the parent heteroclitic BCMA₇₂₋₈₀ (YLMFLLRKI (SEQ ID NO: 1)) peptide utilized as the source of antigen in establishment of the iPSC. Furthermore, we investigated the specific T cell receptor (TCR) repertoire used by the BCMA-specific iPSC-T cells by performing single cell-based sequencing in complementarity-determining region 3 (CDR3) of the Tetramer⁺ cells. Evaluation of 88 single cells revealed a clonotype of TCR alpha and TCR beta sequences, TRAV12-1/TRAJ8 (CVVNIVLGFQKLVF (SEQ ID NO: 9)) paired with beta TRBV20-1/TRBJ2-7 (CSARDSGLPGYEQYF (SEQ ID NO: 10)) (FIG. 7D). Therefore, these results indicate that the BCAM-specific iPSC-CD8⁺CTL generated in these studies have a distinct TCR clonotype, allowing for recognition of their cognate BCMA peptide (YLMFLLRKI (SEQ ID NO: 1)).

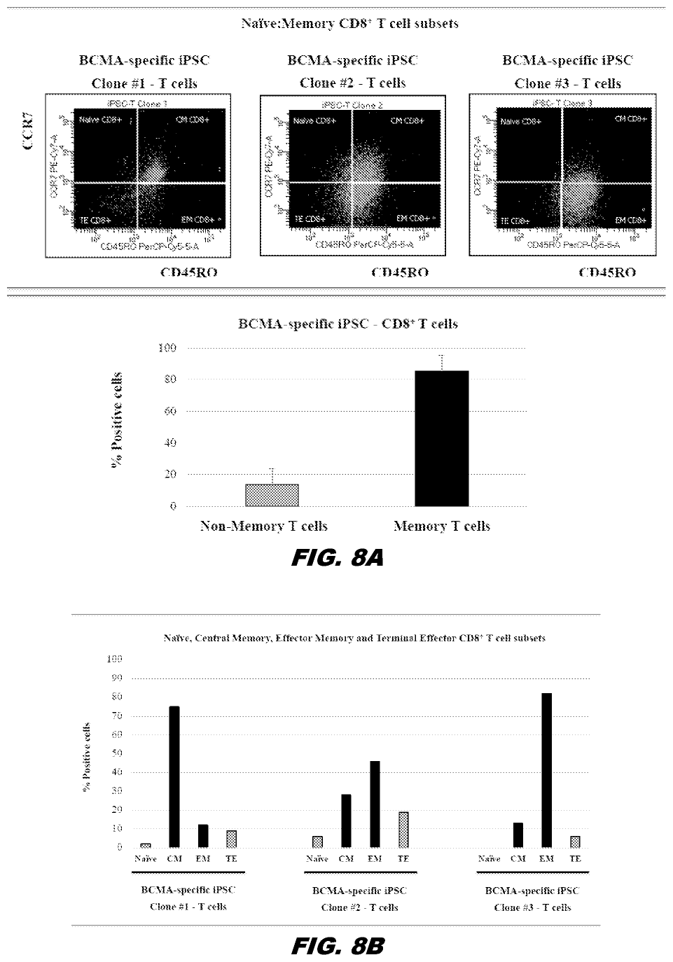

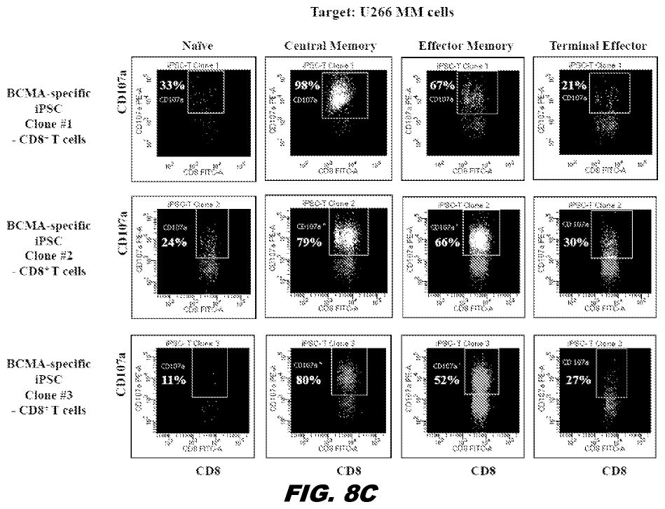

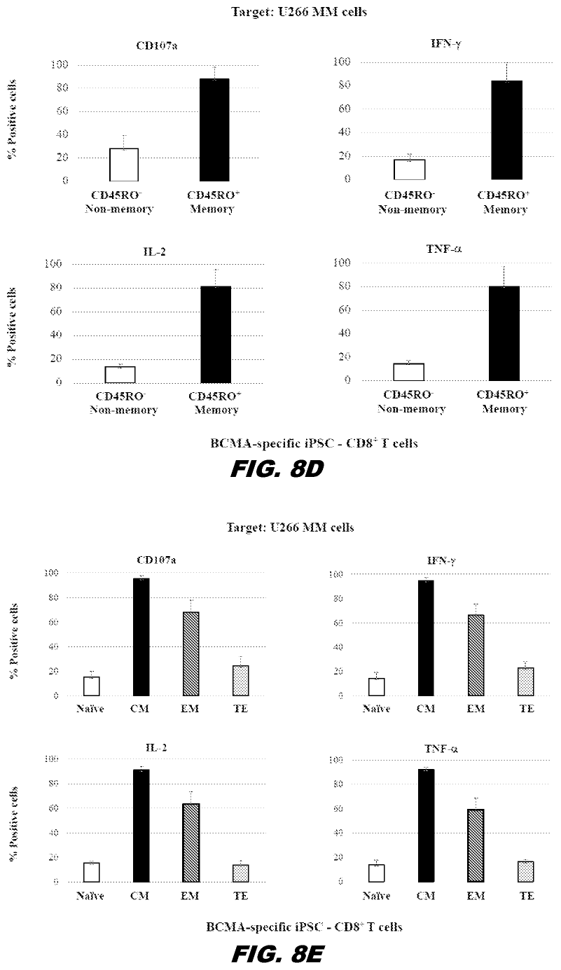

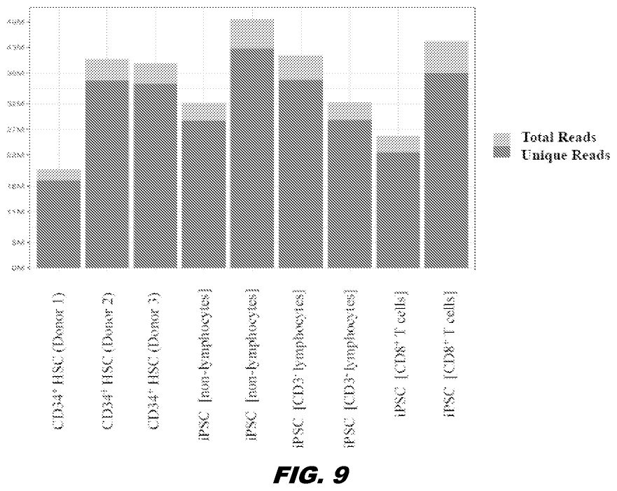

FIG. 8A-8E. A Majority of BCMA-Specific iPSC-T Cells are Memory CD8⁺CTL with Highly Specific Immune Responses with Anti-Tumor Activities Against Myeloma.

We further characterized the BCMA-specific iPSC-T cells for their specific cell subsets within the CD8⁺CTL. It revealed that they were predominantly CD45RO⁺ memory T cells, upon the differentiation from iPSC clone 1, clone 2 and clone 3 (FIG. 8A; dot plots, bar graph). In addition, the memory CD8⁺CTL consisted of both central memory (CM; CCR7⁺CD45RO⁺) and effector memory (EM; CCR7⁻ CD45RO⁺) T cells in the BCMA-specific iPSC-T cells (N=3) with a low frequency of naïve cells (<3%) and terminal effector cells (<10%) (FIG. 8A, 8B). Next, each subset of the Naïve: Memory cells in BCMA-specific iPSC-T cells were investigated for their functional anti-tumor activity and immune response to MM cells. While a low level of anti-tumor activity was detected by naïve and terminal effector CTL within CD45RO⁻ non-memory CTL, a high level of functionality and specificity was found by central memory and effector memory CTL in response to HLA-A2⁺ MM cells (U266). Among the cell populations investigated, the highest anti-MM activity was detected by central memory CTL (79%˜98%), evidenced by CD107a⁺ degranulation, when they were derived from BCMA-iPSC clone #1, #2 or #3, as compared to the effector memory CTL subset (52%˜67%) (FIG. 8C). Furthermore, the summary analyses of BCMA-specific iPSC-T cells differentiated from four separate iPSC clones (N=4) confirmed a significantly (*p<0.05) greater increases in anti-MM activities by CD45RO⁺ memory CTL compared to CD45RO⁻ non-memory CTL as the highest anti-MM activity by central memory CTL subset, as demonstrated by CD107a degranulation (cytotoxicity) and production of Th-1 cytokines (IFN-γ, IL-2, TNF-α) (FIG. 8D). Taken together, these results demonstrate that rejuvenated CD8⁺CTL differentiated from BCMA-specific iPSC clones were highly functional memory T cells with significant (*p<0.05) levels of anti-MM activities including proliferation, degranulation and Th-1 cytokines production in antigen-specific manner (FIG. 8E), thereby providing evidence of their therapeutic potential to treat the patients with myeloma.

### Example 3

Rejuvenated BCMA-Specific CD8+ Cytotoxic T Lymphocytes Derived from Induced Pluripotent Stem Cells for Treatment ofMyeloma

Abstract: This study reports on the reprogramming of B-Cell Maturation Antigen (BCMA)-specific CD8⁺ cytotoxic T lymphocytes (CTL) to induced pluripotent stem cells (iPSC) and their differentiation into rejuvenated antigen-specific CD8⁺CTL as a potential therapeutic option to effectively treat cancer patients. Along with characterization of BCMA-specific iPSC by key stem cell markers, pluripotency potential and normal karyotypes, we further detected their polarization into mesoderm development associated with activation of transcriptional regulators SNAI2, TBX3, PLVAP, HANDI and CDX2 during embryoid body formation. BCMA-specific iPSC clones utilized distinctive commitment pathways during T cells re-differentiation. RNAseq analyses of the iPSC committed to rejuvenated memory CD8⁺ T cells showed unique transcriptional profiles as evidenced by upregulation of transcriptional regulators determining CD4/CD8 T cell differentiation ratio, memory CTL formation, NF-kappa-B/INK pathway activation, and cytokine transporter/cytotoxic mediator development. In parallel, regulators controlling B and T cell interactions or CD4⁺ Th cells and inhibitory receptor development were downregulated. The rejuvenated CD8⁺ BCMA-specific CTL re-differentiated from the iPSC demonstrated (1) mature T cell phenotype and highly enriched central and effector memory T cells without induction of checkpoint molecules; (2) high proliferation and poly-functional anti-myeloma activities in an antigen-specific and HLA-A2-restricted manner; (3) specific immune recognition of cognate HLA-A2 heteroclitic BCMA₇₂₋₈₀ (YLMFLLRKI (SEQ ID NO: 1) peptide; and (4) distinct sole clonotype for T cell receptor. Furthermore, the specific iPSC clones maintained their differentiation potential into CD8⁺ T cells upon sub-cloning or long-term culture under feeder-free culture conditions. In conclusion, these results establish a framework for iPSC-based regenerative medicine to provide rejuvenated and highly functional memory CD8⁺ BCMA-specific CTL as an adoptive immunotherapy to improve patient outcome in multiple myeloma.

Introduction: Engineered T cells generated from cancer patients' T cells can exhibit an “exhausted” phenotype after manipulation ex vivo expansion and multiple efforts are ongoing to select for early lineage central memory cells and thereby prolong clinical responses. One strategy for reversal of T cell exhaustion under evaluation is reprograming to early stage of memory T cells with selective anti-tumor functional activities. A method to overcome T cell exhaustion and muted functional anti-tumor responses is exploitation of fully rejuvenated CTL developed from iPSC. T cell regenerative medicine involving rejuvenation of antigen-specific CD8⁺CTL has the potential to effectively treat patients with cancer uniquely or overexpressing selective antigen. We have developed a cellular reprogramming strategy utilizing TAA-specific CD8⁺ CTL generated by re-differentiation from antigen-specific iPSC. We demonstrate technology for reprogramming BCMA-specific iPSC expressing stem cell markers, pluripotency potential and normal karyotypes, with some of the iPSC clones committed to antigen-specific CD8⁺ CTL with mature T cell phenotype. Here, we report our findings on the respective cell differentiation pathway of iPSC clones and their genetic mechanisms including regulatory elements, which play roles on specific cell commitment into CD8⁺CTL. This study reveals the distinct transcriptional profiles of the iPSC, which polarize into specific cell subset along with their respective genetic regulations on activation and repression sites, providing important information on how to orient and direct the iPSC to differentiate toward specific pathway, especially to generate into TAA-specific CD8⁺CTL as our therapeutic consideration.

Conclusion: The cellular technology developed in this study may allow for the establishment of antigen-specific memory CTL derived from BCMA-iPSC for adoptive immunotherapy to improve clinical outcome in MM. This approach can be beneficial for current clinical protocols and provide a promise for self-renewal and pluripotency for the antigen-specific T cell therapies, especially applying the rejuvenated memory CD8⁺ T cells with a high proliferative capacity and effective anti-tumor activities, thus increase therapeutic efficacy of cancer immunotherapy and effectively treat the patients with cancer.

### Example 4

T cell regenerative medicine represents an emerging immunotherapeutic approach using antigen-specific Induced Pluripotent Stem Cells (iPSC) to rejuvenate CD8⁺ cytotoxic T lymphocytes (CTL). Here, we report on an iPSC-derived therapeutic strategy targeting B-Cell Maturation Antigen (BCMA) against multiple myeloma (MM) via establishment of antigen-specific iPSC, followed by differentiation into highly functional BCMA-specific CD8⁺CTL. The reprogrammed BCMA-specific iPSC displayed normal karyotypes and pluripotency potential as evidenced by expression of stem cell markers (SSEA-4, TRA1-60) and alkaline phosphatase along with differentiation into three germ layers (Ectoderm, Mesoderm, Endoderm). During embryoid body formation, BCMA-specific iPSC was further polarized into the mesoderm germ layer, evidenced by the activation of SNAI2, TBX3, PLVAP, HAND1 and CDX2 transcriptional regulators. Next, the BCMA-specific iPSC clones committed to CD8⁺ T cell differentiation were characterized by analyzing their hematopoietic progenitor cells (HPC; CD34⁺CD43⁺/CD14⁻ CD235a⁻) for specific transcriptional regulation. RNAseq analyses indicated a low variability and similar profiles of gene transcription within the iPSC clones which are committed to CD8⁺CTL, as compared to increased transcriptional variability with iPSC clones committed to different cell types. The unique transcriptional profiles of the iPSC committed to CD8⁺ T cells included upregulation of transcriptional regulators controlling CD4/CD8 T cell differentiation ratio, memory CTL formation, NF-kappa-B/JNK pathway activation, and cytokine transporter/cytotoxic mediator development as well as downregulation of regulators controlling B and T cell interactions and CD4⁺ Th cells and inhibitory receptor development. Specifically, a major regulatory shift, indicated by upregulation of specific genes involved in immune function, was detected in HPC from the iPSC committed to CD8⁺ T cells. BCMA-specific T cells differentiated from the iPSC (referred to herein as “BCMA iPSC-T cells”) were characterized as displaying mature CTL phenotypes including high expression of CD3, CD8α, CD8β, TCRαβ, CD7 along with no CD4 expression. In addition, the final BCMA iPSC-T cells were predominantly CD45RO⁺ memory cells (central memory and effector memory cells) expressing high level of T cell activation (CD38, CD69) and costimulatory (CD28) molecules. Importantly, the BCMA iPSC-T cells lacked immune checkpoints (CTLA4, PD1, LAG3, Tim3) expression and regulatory T cells induction, which are distinct from other antigen-stimulated T cells. The rejuvenated BCMA iPSC-T cells demonstrated a high proliferative (1,000×) during T cell differentiation, poly-functional anti-tumor activities and Th1 cytokine (IFN-γ, IL-2, TNF-α) production in response to MM patients' cells in HLA-A2-restricted manner. Furthermore, the immune responses induced by BCMA iPSC-T cells were specific to the parent heteroclitic BCMA₇₂₋₈₀ (YLMFLLRKI (SEQ ID NO: 1)) peptide, which was used to reprogram and establish the antigen-specific iPSC. Evaluation of 88 single cell Tetramer⁺ CTL from the BCMA iPSC-T cells revealed a clonotype of unique T cell receptor (TCRα, TCRβ) sequence. The BCMA-specific iPSC clones maintained their specific differentiation potential into the antigen-specific CD8⁺ memory T cells, following multiple subcloning in long-term cultures under feeder-free conditions or post-thaw after long-term (18 months) cryopreservation at −140° C., which provide additional benefits for clinical application to treat patients in a continuous manner. Taken together, rejuvenated CD8⁺CTL differentiated from BCMA-specific iPSC were highly functional with significant (*p<0.05) levels of anti-MM activities including proliferation, cytotoxic activity and Th-1 cytokine production to tumor. Therefore, the antigen-specific iPSC reprogramming and T cells rejuvenation process described here can provide an effective and long-term therapeutic efficacy in patients with the antigen-specific memory CTL lacking immune checkpoints and suppressors, providing evidence of their potential for adoptive immunotherapy to improve patient outcome in MM.

### Example 5

Rejuvenated BCMA-Specific iPSC-T Cells Demonstrate Highly Proliferative and Anti-Tumoractivities to MM in Antigen-Specific and HLA-A2-Restricted Manners.

Rejuvenated BCMA-specific iPSC-T cells were analyzed for their poly-functional immune responses against tumor cells. In CFSE-based proliferation assay, the iPSC-T cells displayed a higher level of T cells proliferation in response to BCMA+HLA-A2+U266 MM cells (CD3+: 94%, CD8+: 97%) as compared to MHC mis-matched BCMA+HLA-A2-RPMI MM cells (CD3+: 2%, CD8+: 4%) or antigen mis-matched BCMA-HLA-A2+MDA-MB231 breast cancer cells (CD3+: 3%, CD8+: 0%) (FIG. 6A). These results confirm that rejuvenated BCMA-specific iPSC-T cells maintained cognate BCMA antigen-specific and HLA-A2-restricted recognition of tumor cells. Next, we investigated the BCMA-specific iPSC-T cells for their specific cytotoxic activities and Th1-type cytokine production against MM cells and detected HLA-A2-restricted anti-tumor activities and specific immune responses, as indicated by CD107a degranulation (49%) as well as production of IFN-γ (50%), IL-2 (47%) and TNF-α (47%) in response to U266 MM cells [BCMA+HLA-A2+], but not to HLA-A2 mis-matched RPMI MM cells [BCMA+-HLA-A2-](FIG. 6B). In addition, we analyzed T cells differentiated from the respective BCMA-specific iPSC clones (N=3) and detected a consistent increases (*p>0.05) in their anti-tumor activities and IFN-γ/IL-2/TNF-production, specifically to the BCMA+/ HLA-A2+U266 MM cells, but not to BCMA+/HLA-A2 mis-matched RPMI MM cells nor antigen mis-matched/HLA-A2+MDA-MB231 breast cancer cells, as the similar levels to the baseline (no stimulation with tumor cells) (<5%) (FIG. 6D). The functional activities of BCMA-specific iPSC-T cells were further investigated against primary CD138⁺ tumor cells isolated from MM patients. The BCMA-specific iPSC-T cells displayed robust anti-MM activities against primary CD138+ tumor cells from HLA-A2+MM patient A (FIG. 6E) or patient B (FIG. 6F) as measured by CD107a degranulation and TNF-production [iPSC clone #1: CD107a+: 57% or 59%, TNF-α+: 44% or 36%; iPSC clone #2: CD107a+: 42% or 43%, TNF-α+: 25% or 27%]. However, the T cells did not respond to HLAA2⁻ MM Patient C nor Patient D (<5%) primary MM tumor cells (FIGS. 6E and 6F). The HLA-A2 restricted anti-MM activities were consistently observed in the iPSC-T cells differentiated from either iPSC clone #1 or iPSC clone #2, evidenced by distinct responses to CD138+ tumor cells from HLA-A2+MM patients (N=3), but not to those from HLA-A2-MM patients (N=3) (FIG. 6G). Thus, these data demonstrate that the processes outlined in this study allows generation of rejuvenated BCMA-specific T cells with high levels of anti-MM activities, evidenced by their high proliferation capacity, CD107a degranulation and ThI-type cytokine production, further supporting their potential therapeutic application to treat MM patients.

Rejuvenated BCMA-Specific iPSC-T Cells Demonstrate Peptide-Specific Immune Responses to the Cognate Heteroclitic BCMA₇₂₋₈₀ (YLMFLLRKI (SEQ ID NO: 1)) Peptide and Display a Sole Distinct TCR Clonotype.

Following the confirmation of anti-tumor activities of BCMA-specific iPSC-T cells against MM cells in HLA-A2-restricted manner, we further investigated their specificity and ability to recognize and respond to the cognate heteroclitic BCMA₇₂₋₈₀ (YLMFLLRKI (SEQ ID NO: 1) peptide on antigenpresenting cells or MM cells. CFSE-based assays were performed to measure the specific proliferation of BCMA iPSC-T cells in response to relevant (BCMA-derived) or irrelevant (HIV-derived) peptide. Representative flow cytometric analyses showed minimum levels of CD3+ T cells proliferation (5˜7%) in response to the antigen-presenting cells (APC; T2 or K562-A*0201) alone or APC loaded with irrelevant HLA-A2-specific HIV-Gag₇₇₋₈₅ peptide (SLYNTVATL (SEQ ID NO: 2)) (FIG. 7A). Importantly, the BCMA-specific iPSC-T cells demonstrated a high CD3+ T cell proliferation in response to heteroclitic BCMA₇₂₋₈₀ (YLMFLLRKI (SEQ ID NO: 1)) loaded T2 cells (45%) or K562-A*0201 cells (40%), measured on day 6 of co-culture. Specific response of BCMA iPSCT cells to the cognate BCMA peptide emerged in a time-dependent manner as a gradual increase in their CD8+ T cells proliferation on day 5 (14%), day 6 (32%) and day 7 (81%) compared to baseline of T2 cells alone (2˜7%) (FIG. 7B). The specificity of BCMA iPSC-T cells was further investigated in response to HLA-A2+U266 MM cells pulsed with heteroclitic BCMA₇₂₋₈₀ peptide. The proliferation level of T cells (CD3+, CD8+) was increased in response to BCMA₇₂₋₈₀ peptide pulsed U266 MM cells compared to U266 MM cells alone. They demonstrated a gradual increase in T cell proliferation over time on day 4 (stimulator; no peptide pulsed U266 vs. BCMA peptide pulsed U266: 25% vs. 34% CD3+ T cells), day 5 (52% vs. 73% CD3+ T cells) and day 6 (66% vs. 92% CD8+ T cells) (FIG. 7C). Taken together, these data indicate that the BCMAspecific iPSC-T cells are capable of recognizing and responding to the parent heteroclitic BCMA₇₂₋₈₀ (YLMFLLRKI (SEQ ID NO: 0) peptide used to establish BCMA-specific iPSC. Finally, we determined the specific T cell receptor (TCR) on the BCMA-specific iPSC-T cells using a single cell-based sequencing of FACS sorted heteroclitic BCMA₇₂₋₈₀ (YLMFLLRKI (SEQ ID NO: 1)) peptide-specific Tetramer+/CD8+ cells. Evaluation of 88 single cells revealed a clonotype of T cell receptor TCRα and TCRβ sequences, TRAV12-1/TRAJ8/CVVNIVLGFQKLVF (SEQ ID NO: 9) paired with beta TRBV20-1/TRBJ2-7/CSARDSGLPGYEQYF (SEQ ID NO: 10) (FIG. 7D). These results indicate that the BCAM-specific iPSC-T cells generated in these studies have a sole TCR clonotype, allowing for recognition of the cognate heteroclitic BCMA₇₂₋₈₀ peptide.

Rejuvenated BCMA-Specific iPSC-T Cells are “Memory CD8+CTL” with Highly Specific Immune Responses and Anti-Tumor Activities Against Myeloma.

Finally, we characterized the BCMA-specific iPSC-T cells for their specific memory CD8+ T cell subsets. The BCMA-specific iPSC-T cells were predominantly CD45RO+ memory cells upon redifferentiation of T cells from the different iPSC clones (N=3; FIG. 8A; dot plots, bar graph). Specifically, they consisted of both central memory (CM; CCR7+CD45RO+) and effector memory (EM; CCR7−CD45RO+) cells and a corresponding low frequency of naïve cells (<3%) and terminal effector cells (<10%) (FIGS. 8A and 8B). Next, the Naïve: Memory CD8+ T cell subsets were evaluated for their functional anti-MM activities. Overall, we observed a high level of functionality and specificity within the central memory and effector memory T cell subsets in response to HLA-A2+MM cells (U266), while a low level of anti-tumor activity was detected by naïve and terminal effector T cell subsets within CD45RO− non-memory CTL. Among the cell subsets investigated, the highest anti-MM activity was detected by central memory CTL (79%˜98%) derived from BCMA-iPSC clone #1, #2 or #3, evidenced by CD107a+ degranulation, as compared to the effector memory CTL subset (52%˜67%) (FIG. 8C). Finally, BCMA-specific iPSC-T cells differentiated from three different iPSC clones (N=3) showed significantly (*p<0.05) greater immune responses within the CD45RO+ memory cells compared to CD45RO− non-memory cells (FIG. 8D), as demonstrated by CD107a degranulation (cytotoxicity) and production of Th-1 cytokines (IFN-γ, IL-2, TNF-α which demonstrated the highest anti-MM activity by central memory CTL subset (FIG. 8E). Taken together, these results demonstrate that “rejuvenated” CD8+CTL differentiated from BCMA-specific iPSC clones consisted of highly functional memory T cells having elevated levels (*p<0.05) of anti-MM activities including proliferation, CD107a degranulation and Th-1 cytokine production, thereby providing evidence of their therapeutic potential to treat the patients with myeloma.

### EQUIVALENTS

Those skilled in the art will recognize, or be able to ascertain, using no more than routine experimentation, numerous equivalents to the specific substances and procedures described herein. Such equivalents are considered to be within the scope of this invention.

1. An induced pluripotent stem cell (iPSC) that re-differentiates to at least one of a CD8⁺ cytotoxic T lymphocyte (CTL) (iPSC [CD8⁺ T cell]), a lymphocyte that does not express CD3 (iPSC [CD3⁻ lymphocyte]) or a non-lymphocyte (iPSC [non-lymphocyte]).

2. The induced pluripotent stem cell (iPSC) of claim 1 that re-differentiates to one of a CD8⁺CTL (iPSC [CD8⁺ T cell]), a lymphocyte that does not express CD3 (iPSC [CD3⁻ lymphocyte]) or a non-lymphocyte (iPSC [non-lymphocyte]).

3. induced pluripotent stem cell (iPSC) of claim 1 that re-differentiates to a CD8⁺CTL (iPSC [CD8⁺ T cell]) and not to a lymphocyte that does not express CD3 (iPSC [CD3⁻ lymphocyte]) or a non-lymphocyte (iPSC [non-lymphocyte]).

4. An induced pluripotent stem cell (iPSC) that re-differentiates to a lymphocyte that does not express CD3 (iPSC [CD3⁻ lymphocyte]) and not to a CD8⁺CTL (iPSC [CD8⁺ T cell]) or a non-lymphocyte (iPSC [non-lymphocyte]).

5. An induced pluripotent stem cell (iPSC) that re-differentiates to a non-lymphocyte (iPSC [non-lymphocyte]) and not to a CD8⁺CTL (iPSC [CD8⁺ T cell]) or a lymphocyte that does not express CD3 (iPSC [CD3⁻ lymphocyte]).

6. The iPSC of claim 1, wherein the iPSC comprises an iPSC [CD8⁺ T cell] that is specific for an antigen.

7.-10. (canceled)

11. The iPSC of claim 1, wherein the iPSC is re-programmed from a CD8⁺CTL that is specific for an antigen.

12.-14. (canceled)

15. The iPSC of claim 1, wherein the iPSC has a normal karyotype, expresses SSEA-4 and TRA-1-60, differentiates into ectoderm, mesoderm and endoderm, retains alkaline phosphate during colony formation, or a combination thereof.

16. The iPSC of claim 1, wherein the iPSC that re-differentiates to a CD8⁺ CTL (iPSC [CD8⁺ T cell]) has increased expression of at least 2, 4, 6, 8, 10 or 12 of genes FOXF1, GZMB, ITGA1, TBX3, MX1, TNFRSF9, CD1A, LCK, LTB, IFIT3, TNFSF10 and A2M as compared to the iPSC that re-differentiates to the lymphocyte that does not express CD3 (iPSC [CD3⁻ lymphocyte]).

17. The iPSC of claim 1, wherein the iPSC that re-differentiates to a CD8⁺CTL (iPSC [CD8⁺ T cell]) has decreased expression of at least 1, 2 or 3 of genes TGFBR3, CD37 and SlPRl as compared to the iPSC that re-differentiates to the lymphocyte that does not express CD3 (iPSC [CD3⁻ lymphocyte]).

18. The iPSC of claim 1, wherein the iPSC that re-differentiates to a CD8⁺CTL (iPSC [CD8⁺ T cell]) has increased expression of at least 1, 3, 5, or 7 of genes TBX3, ZNF683, FOXF1, GZMB, IL7R, A2M and SORL1 as compared to the iPSC that re-differentiates to the non-lymphocyte (iPSC [non-lymphocyte]).

19. The iPSC of claim 1, wherein the iPSC that re-differentiates to a CD8⁺CTL (iPSC [CD8⁺ T cell]) has decreased expression of at least 1, 3, 5, or 7 of genes TGFBR3, GDF3, BLNK, FRRS1, KLF2, NCF2 and KDR as compared to the iPSC that re-differentiates to the non-lymphocyte (iPSC [non-lymphocyte]).

20. The iPSC of claim 1, wherein the iPSC that re-differentiates to a CD8⁺CTL (iPSC [CD8⁺ T cell]) has increased expression of at least 2, 4, 6, 8, 10 or 12 of genes CX3CR1, CD3D, CD1A, CDH5, ILR7, PLVAP, LEF1, A2M, NCR2, CCNB2, ORC6 and NUSAP1 as compared to hematopoietic progenitor cells (HPC), which are CD34⁺CD43⁺/CD14⁻ CD235a⁻, from the iPSC.

21. The iPSC of claim 1, wherein the iPSC that re-differentiates to a CD8⁺CTL (iPSC [CD8⁺ T cell]) has decreased expression of at least 2, 4, or 6 of genes DNTT, LAG3, KLF2, CD37, SELL and SORL1 as compared to hematopoietic progenitor cells (HPC), which are CD34⁺CD43⁺/CD14⁻ CD235a⁻, from the iPSC.

22. The iPSC of claim 1, wherein the iPSC that re-differentiates to a CD8⁺CTL (iPSC [CD8⁺ T cell]) has increased expression of genes:
TBX3, HOXA11, IRF4, PIK3C2B, KLF15, IL-12B, MAPK4, ITLN 1/2, TRIM6, EDA2R;
as compared to the iPSC that re-differentiates to the lymphocyte that does not express CD3 (iPSC [CD3⁻ lymphocyte]) and the iPSC that re-differentiates to the non-lymphocyte (iPSC [non-lymphocyte]).

23. The iPSC of claim 1, wherein the iPSC that re-differentiates to a CD8⁺CTL (iPSC [CD8⁺ T cell]) has decreased expression of genes:
RPS6KA2, CDK3, YEPL4, BATF2, BTN3A1, BTN3A1, USP44, CD70, ZXDA, FGFR1, NPM2, GGN, SPAG1, CATSPER2, N4BP3, P2RY14, NLGN2, SHC2, GRASP, AMIGO2, TBC1D32, CACNA1A, SLC6A9, HEYL, NEURL, RAB39B, ANK1, PSD, LRRK1, RUNX2, CXCL5, SEMA7A, JDP2, PLA2G6, MAP3K9, PIPOX, TNFRSF6B;
as compared to the iPSC that re-differentiates to the lymphocyte that does not express CD3 (iPSC [CD3⁻ lymphocyte]) and the iPSC that re-differentiates to the non-lymphocyte (iPSC [non-lymphocyte]).

24. The iPSC of claim 1, wherein a cytotoxic T cell (CTL) re-differentiated from the iPSC has an antigen-specific, MHC-restricted proliferation response.

25. The iPSC of claim 1, wherein a cytotoxic T cell (CTL) re-differentiated from the iPSC has an antigen-specific, MHC-restricted cytotoxic response.

26. The iPSC of claim 1, wherein a cytotoxic T cell (CTL) re-differentiated from the iPSC comprises a memory CD8⁺CTL (CD45RO⁺).

27. (canceled)

28. (canceled)

29. The iPSC of claim 1, wherein a cytotoxic T cell (CTL) re-differentiated from the iPSC comprises a non-memory CD8⁺CTL (CD45RO⁻).

30. A CD8⁺CTL cell re-differentiated from the induced pluripotent stem cell (iPSC) of claim 1.

31. A CD8⁺ CTL cell re-differentiated from an induced pluripotent stem cell (iPSC), wherein the iPSC is re-programmed from a cytotoxic T lymphocyte (CTL) that is specific for a tumor-associated antigen (TAA), tumor-specific antigen (TSA) or a B-cell maturation antigen (BCMA).

32. (canceled)

33. An iPSC from which the CD8⁺CTL cell of claim 31 is re-differentiated, wherein the iPSC:
has increased expression of at least 2, 4, 6, 8, 10 or 12 of genes FOXF1, GZMB, ITGA1, TBX3, MX1, TNFRSF9, CD1A, LCK, LTB, IFIT3, TNFSF10 and A2M as compared to an iPSC that re-differentiates to a lymphocyte that does not express CD3 (iPSC [CD3⁻ lymphocyte]);
has decreased expression of at least 1, 2 or 3 of genes TGFBR3, CD37 and S1PR1 as compared to an iPSC that re-differentiates to a lymphocyte that does not express CD3 (iPSC [CD3⁻ lymphocyte]);
has increased expression of at least 1, 3, 5, or 7 of genes TBX3, ZNF683, FOXF1, GZMB, IL7R, A2M and SORL1 as compared to an iPSC that re-differentiates to a non-lymphocyte (iPSC [non-lymphocyte]);
has decreased expression of at least 1, 3, 5, or 7 of genes TGFBR3, GDF3, BLNK, FRRS1, KLF2, NCF2 and KDR as compared to an iPSC that re-differentiates to a non-lymphocyte (iPSC [non-lymphocyte]);
has increased expression of at least 2, 4, 6, 8, 10 or 12 of genes CX3CR1, CD3D, CD1A, CDH5, ILR7, PLVAP, LEF1, A2M, NCR2, CCNB2, ORC6 and NUSAP1 as compared to hematopoietic progenitor cells (HPC), which are CD34⁺CD43⁺/CD14⁻ CD235a⁻, from the iPSC;
has decreased expression of at least 2, 4, or 6 of genes DNTT, LAG3, KLF2, CD37, SELL and SORL1 as compared to hematopoietic progenitor cells (HPC), which are CD34⁺CD43⁺/CD14⁻ CD235a⁻, from the iPSC;
has increased expression of genes TBX3, HOXA11, IRF4, PIK3C2B, KLF15, IL-12B, MAPK4, ITLN 1/2, TRIM6, EDA2R as compared to an iPSC that re-differentiates to a lymphocyte that does not express CD3 (iPSC [CD3⁻ lymphocyte]) and an iPSC that re-differentiates to a non-lymphocyte (iPSC [non-lymphocyte]); or
has decreased expression of genes RPS6KA2, CDK3, YEPL4, BATF2, BTN3A1, BTN3A1, USP44, CD70, ZXDA, FGFR1, NPM2, GGN, SPAG1, CATSPER2, N4BP3, P2RY14, NLGN2, SHC2, GRASP, AMIGO2, TBC1D32, CACNA1A, SLC6A9, HEYL, NEURL, RAB39B, ANK1, PSD, LRRK1, RUNX2, CXCL5, SEMA7A, JDP2, PLA2G6, MAP3K9, PIPOX, TNFRSF6B as compared to an iPSC that re-differentiates to a lymphocyte that does not express CD3 (iPSC [CD3⁻ lymphocyte]) and an iPSC that re-differentiates to a non-lymphocyte (iPSC [non-lymphocyte]).

34. (canceled)

35. A composition, comprising:
the induced pluripotent stem cell (iPSC) of claim 1; and/or
a CD8⁺CTL redifferentiated from the iPSC of claim 1.

36. A composition comprising an induced pluripotent stem cell (iPSC) re-programmed from a cytotoxic T cell (CTL), wherein the CTL is specific for a tumor antigen, wherein the iPSC can be re-differentiated into a genetically modified immune cell.

37.-63. (canceled)

64. A method of treating a cancerous or precancerous condition in a subject, the method comprising administering to a subject in need thereof an effective amount of the induced pluripotent stem cell (iPSC) of claim 1, thereby treating cancer in a subject.

65.-39. (canceled)
